(19) United States
(12) Patent Application Publication
(10) Pub. No.: US 2021/0298340 A1
(43) Pub. Date: Sep. 30, 2021

(54) Cannabis-Infused Product with Extended Cannabinoid Profile User Experience

(71) Applicant: HEXO Operations Inc., Ottawa (CA)

(72) Inventors: Max ALSAYAR, Gatineau (CA); Francois CHOUINARD, Gatineau (CA); Justin CONWAY, Gatineau (CA); Denis KESERIS, Montreal (CA); George ELVIRA, Montreal (CA); Stephan GEORGIEV, Montreal (CA); Walter CHAN, Montreal (CA)

(21) Appl. No.: 17/269,940

(22) PCT Filed: Aug. 20, 2019
(86) PCT No.: PCT/CA2019/051135

§ 371 Date: Feb. 19, 2021

Related U.S. Application Data
(60) Provisional application No. 62/725,308, filed on Aug. 31, 2018.
(60) Provisional application No. 62/725,142, filed on Aug. 30, 2018.
(60) Provisional application No. 62/722,422, filed on Aug. 24, 2018.
(60) Provisional application No. 62/719,926, filed on Aug. 20, 2018.

(51) Int. Cl. A23L 33/105; A61K 31/352; A61K 45/06; A61K 9/107; A61K 9/00; A61K 9/68; A23D 7/005; A23L 2/52

(52) U.S. Cl. CPC A23L 33/105; A61K 31/352; A61K 45/06; A61K 9/107; A23V 2002/00; A61K 9/0095; A61K 9/0058; A23D 7/0053; A23L 2/52; A61K 9/0056

(57) ABSTRACT

The present disclosure relates to a cannabis-infused product comprising a cannabinoid profile including one or more cannabinoid, a first composition for controlling onset of the cannabinoid profile and a second composition for extending offset of the cannabinoid profile in a subject having used the cannabis-infused product, wherein the second composition has a delayed onset compared to that one of the first composition. The present disclosure also relates to methods of manufacture and of using same.

### CROSS-REFERENCE TO RELATED APPLICATIONS

The present application claims the benefit of U.S. provisional patent application serial number U.S. 62/719,926 filed on Aug. 20, 2018, U.S. provisional patent application serial number U.S. 62/722,422 filed on Aug. 24, 2018, U.S. provisional patent application serial number U.S. 62/725,142 filed on Aug. 30, 2018, and U.S. provisional patent application serial number U.S. 62/725,308 filed on Aug. 31, 2018. The contents of each of the above-referenced documents are incorporated herein by reference in their entirety.

### TECHNICAL FIELD

The present application relates to a cannabis-infused product having an extended cannabinoid profile user experience as well as methods of manufacturing and of using same.

### BACKGROUND

As the cannabis industry grows and an increasing number of new products are available for consumers, it has become paramount to produce a product that is safe and well controlled in experience for the consumer. One of the main aspects of this being a predictable user experience. Present cannabis-infused products (such as edibles, topicals or beverages) are often criticized as having significant unpredictability in terms of on-set, with a marked disparity of up to 2 hours between individuals who have consumed the same product and quantity, and in terms of longevity of cannabis-associated effect with a marked disparity of up to 6-8 hours for solid edibles and 2-4 hours for sublingual liquid compositions.

Consumers often do not understand this aspect of cannabis-infused products use and may consume a greater than intended amount of drug before the drug has taken effect, often resulting in profoundly adverse effects, or may consume less than intended amount of drug resulting in a suboptimal cannabis-associated effect. Although ample experimental evidence demonstrates that cannabis is not particularly lethal and, to date, no deaths have been directly attributed to the acute physical toxicity of cannabis, episodes of severe cannabis-induced behavioral impairment are common, and can result in cognitive and motor impairment, extreme sedation, agitation, anxiety, cardiac stress, and vomiting. Most troubling, high quantities of Δ⁹-THC are reported to produce such transient psychotic symptoms as hallucinations, delusions, and anxiety in some individuals. Further, the amount of CBD required for a given individual to perceive at least one of a neuroprotective, antiepileptic, anxiolytic, antipsychotic, analgesic, or anti-inflammatory effect may vary between individuals, but more importantly, because of the delay in onset or the variability in terms of longevity of the cannabis-associated effect, the user may consume less than intended amount of CBD resulting in a suboptimal cannabis-associated effect and/or perception that the CBD-infused product does not work.

Moreover, the amount of Δ⁹-THC in cannabis-infused products can vary across a single product and across batches formulated at different times, making it difficult for users to estimate how much Δ⁹-THC they consume. The lack of consistency and the delayed intoxication has also been reported with use of other cannabis-infused products containing various cannabinoid profiles and may cause both new and experienced users of cannabis to consume higher than intended amounts of the cannabinoid contained in the cannabinoid profile.

The cannabis industry thus faces significant challenges in view of such problems and risks having consumers demand alternative solutions perceived as being less risky, which could have significant commercial impacts.

### SUMMARY

This Summary is provided to introduce a selection of concepts in a simplified form that are further described below in the Detailed Description. This Summary is not intended to identify key aspects or essential aspects of the claimed subject matter.

As embodied and broadly described herein, the present disclosure relates to a cannabis-infused product, comprising a cannabinoid profile including one or more cannabinoid, a first composition for controlling onset of the cannabinoid profile and a second composition for extending offset of the cannabinoid profile in a subject having used the cannabis-infused product, wherein the second composition has a delayed onset compared to that one of the first composition, wherein the cannabis-infused product includes a non-liquid edible matrix.

As embodied and broadly described herein, the present disclosure also relates to a cannabis precursor composition for infusing a product base so as to obtain a non-liquid edible matrix cannabis-infused product, the precursor composition comprising a cannabinoid profile including one or more cannabinoid, a first composition for controlling onset of the cannabinoid profile and a second composition for extending offset of the cannabinoid profile in a subject having used the cannabis-infused product, wherein the second composition has a delayed onset compared to that one of the first composition.

As embodied and broadly described herein, the present disclosure also relates to a cannabis-infused product, comprising a cannabinoid profile including one or more cannabinoid, a first composition for controlling onset of the cannabinoid profile and a second composition for extending offset of the cannabinoid profile in a subject having used the cannabis-infused product, wherein the second composition has a delayed onset compared to that one of the first composition, the cannabis-infused product being a cannabis-infused liquid composition.

As embodied and broadly described herein, the present disclosure also relates to a cannabis precursor composition for infusing a product base so as to obtain a non-liquid edible matrix cannabis-infused product, the precursor composition comprising a cannabinoid profile including one or more cannabinoid, a first composition for controlling onset of the cannabinoid profile and a second composition for extending offset of the cannabinoid profile in a subject having used the cannabis-infused product, the cannabis-infused product being a cannabis-infused liquid composition.

As embodied and broadly described herein, the present disclosure also relates to a cannabis-infused beverage comprising a cannabinoid profile including one or more cannabinoid, a first emulsion having a flux value of at least 0.05 FU in a Franz cell diffusion test and a second emulsion having a flux value of less than 0.05 FU in the Franz cell diffusion test.

As embodied and broadly described herein, the present disclosure also relates to a method of manufacturing a cannabis-infused product, comprising selecting a cannabinoid profile including one or more cannabinoid, selecting a first emulsion having a first flux value of at least 0.05 FU in a Franz cell diffusion test, and mixing at least a first portion of said cannabinoid profile with said first emulsion to obtain a first precursor composition, selecting a second emulsion having a second flux value of less than 0.05 FU in the Franz cell diffusion test, mixing at least a second portion of said cannabinoid profile with said second emulsion to obtain a second precursor composition, and infusing the first and second compositions with a product base so as to obtain the cannabis-infused product.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific exemplary embodiments is provided herein below with reference to the accompanying drawings in which:

FIG. 1A and FIG. 1B illustrate a non-limiting Franz Diffusion Cell embodiment for the Franz cell test in accordance with an embodiment of the present disclosure;

FIG. 2 illustrates a non-limiting cell permeation experiment embodiment for the Cell permeation test in accordance with an embodiment of the present disclosure;

FIG. 3 shows a graph that illustrates results obtained using THC emulsions having 40 nm, 200 nm and >1000 nm in the Franz Cell test in accordance with an embodiment of the present disclosure;

FIG. 4 illustrates a flow chart for manufacturing a cannabis-infused product in accordance with an embodiment of the present disclosure.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

A detailed description of one or more embodiments of the invention is provided below along with accompanying figures that illustrate the principles of the invention. The invention is described in connection with such embodiments, but the invention is not limited to any embodiment. The scope of the invention is limited only by the claims. Numerous specific details are set forth in the following description in order to provide a thorough understanding of the invention. These details are provided for the purpose of non-limiting examples and the invention may be practiced according to the claims without some or all of these specific details. For the purpose of clarity, technical material that is known in the technical fields related to the invention has not been described in detail so that the invention is not unnecessarily obscured.

The present inventors have surprisingly and unexpectedly discovered that at least some of the problems discussed above with respect to cannabis-infused products may be addressed by providing cannabis-infused products which have a fast onset and an offset of the cannabis-associated effect in a manner which can be more consistent and controlled. Advantageously, use of the herein described cannabis-infused products may provide a cannabis-associated effect that can be extended in time compared to a similar cannabis-infused product but which does not include the benefits from the present disclosure. In order to achieve this, the present specification discloses cannabis-infused products, which are designed to control and/or modulate the onset/offset of the cannabinoid profile contained therein.

Advantageously, it has been observed that such cannabis-infused product may afford an enhanced and more consistent user experience—e.g., one can substantially tailor his/her cannabis user experience by consuming such cannabis-infused product.

In the present specification, the expression “cannabis-infused” will be used with reference to consumer products (such as a topical, edible, beverage, and the like), which contain a cannabis derived compound (such as one or more cannabinoid) as an ingredient component that has been admixed or infused with other ingredients forming the consumer product. For example, in the case of a cannabis-infused beverage product, this beverage product can be made by infusing the herein described composition containing a cannabinoid profile in a beverage base, preferably a cannabinoid-less beverage base. The infusion can be performed by mixing a powdered form of the composition and/or a liquid form of the composition with the beverage base.

In the present specification, the expression “cannabinoid profile” will be used with reference to one or more cannabinoid(s) and amount(s) thereof contained in a particular cannabis-infused product, which are expected to provide a given user experience to a person using the cannabis-infused product. For example, when a cannabis-infused product contains a psychotropic cannabinoid in an amount sufficient to provide a psychotropic user experience to a person having used same (i.e., the person feels “high”), this psychotropic user experience can be referred to as being the cannabis-associated effect which is associated with the “cannabinoid profile”, namely in this case being the presence of the psychotropic cannabinoid in said amount. Similarly, when the cannabis-infused product contains instead an anxiolytic cannabinoid in an amount sufficient to provide an anti-anxiety user experience to a person having used same (i.e., the person feels “less anxious”), this anti-anxiety user experience can be referred to as being the cannabis-associated effect which is associated with the cannabinoid profile, namely in this case being the presence of the anxiolytic cannabinoid in said amount. Various cannabinoid profiles are possible and will be apparent to the person of skill, as such, and for conciseness sake, will not be further described here.

It will also be understood by the reader that the herein discussed cannabinoid profile may also include, in addition to the one or more cannabinoid, one or more terpene, one or more flavonoid, or any combinations thereof.

There are a number of options to obtain the herein described cannabis-infused product.

For example, one can design a cannabis-infused product containing a first composition for controlling onset of the cannabinoid profile and a second composition for extending timing of offset of the cannabinoid profile in a subject having used the cannabis-infused product, where the second composition has a delayed onset compared to that one of the first composition.

For example, one can design a cannabis-infused product containing a fast onset portion of the cannabinoid profile and a delayed onset portion of the particular cannabinoid profile.

For example, one can design a precursor composition, which includes both the fast onset portion and the delayed onset portion, and then infuse a product base with the precursor composition to obtain the cannabis-infused product.

For example, one can design a first precursor composition, which contains the fast onset portion and a second precursor composition, which contains the delayed onset portion, and then infuse the product base with both precursor compositions either concomitantly or sequentially to obtain the cannabis-infused product.

These and other examples of implementation of the present disclosure will become apparent to the person of skill in view of the disclosure as a whole.

### 1. Cannabis

Cannabis is a genus of flowering plants that includes a number of species. The number of species is currently being disputed. There are three different species that have been recognized, namely Cannabis sativa, Cannabis indica and Cannabis ruderalis. Hemp, or industrial hemp, is a strain of the Cannabis sativa plant species that is grown specifically for the industrial uses of its derived products. Hemp has lower concentrations of THC and higher concentrations of cannabidiol (CBD), which decreases or eliminates its psychoactive effects.

The term “Cannabis plant(s)” encompasses wild type Cannabis and also variants thereof, including cannabis chemovars which naturally contain different amounts of the individual cannabinoids. For example, some Cannabis strains have been bred to produce minimal levels of THC, the principal psychoactive constituent responsible for the high associated with it and other strains have been selectively bred to produce high levels of THC and other psychoactive cannabinoids.

Cannabis plants produce a unique family of terpeno-phenolic compounds called cannabinoids, which produce the “high” one experiences from consuming marijuana. There are 483 identifiable chemical constituents known to exist in the cannabis plant, and at least 85 different cannabinoids have been isolated from the plant. The two cannabinoids usually produced in greatest abundance are cannabidiol (CBD) and/or Δ9-tetrahydrocannabinol (THC), but only THC is psychoactive. Cannabis plants are categorized by their chemical phenotype or “chemotype,” based on the overall amount of THC produced, and on the ratio of THC to CBD. Although overall cannabinoid production is influenced by environmental factors, the THC/CBD ratio is genetically determined and remains fixed throughout the life of a plant. Non-drug plants produce relatively low levels of THC and high levels of CBD, while drug plants produce high levels of THC and low levels of CBD.

### 2. Cannabinoid

A cannabinoid is generally understood to include any chemical compound that acts upon a cannabinoid receptor such as CB1 and CB2. A cannabinoid may include endocannabinoids (produced naturally by humans and animals), phytocannabinoids (found in cannabis and some other plants), and synthetic cannabinoids (manufactured artificially).

Examples of phytocannabinoids include, but are not limited to, cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (W-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ⁸-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2, 6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof.

The terms “cannabidiol” or “CBD” are generally understood to refer to one or more of the following compounds, and, unless a particular other stereoisomer or stereoisomers are specified, includes the compound “A-cannabidiol.” These compounds are: (1) Δ⁵-cannabidiol (2-(6-isopropenyl-3-methyl-5-cyclohexen-1-yl)-5-pentyl-1,3-benzenediol); (2) Δ⁴-cannabidiol (2-(6-isopropenyl-3-methyl-4-cyclohexen-1-yl)-5-pentyl-1,3-benzenediol); (3) Δ³-cannabidiol (2-(6-isopropenyl-3-methyl-3-cyclohexen-1-yl)-5-pentyl-1,3-benzenediol); (4) Δ^{3,7}-cannabidiol (2-(6-isopropenyl-3-methylenecyclohex-1-yl)-5-pentyl-1,3-benzenediol); (5) Δ²-cannabidiol (2-(6-isopropenyl-3-methyl-2-cyclohexen-1-yl)-5-pentyl-1,3-benzenediol); (6) Δ¹-cannabidiol (2-(6-isopropenyl-3-methyl-1-cyclohexen-1-yl)-5-pentyl-1,3-benzenediol); and (7) Δ⁶-cannabidiol (2-(6-isopropenyl-3-methyl-6-cyclohexen-1-yl)-5-pentyl-1,3-benzenediol).

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

In some embodiments, the cannabinoid is a mixture of tetrahydrocannabinol (THC) and cannabidiol (CBD). The w/w ratio of THC to CBD in the liquid formulation may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1, or about 1000:1.

### 3. Terpene/Terpenoid

A terpene is generally understood to include any organic compound derived biosynthetically from units of isoprene, and the term “terpenoid” generally refers to a chemically modified terpene (e.g., by oxidation). Terpenes are produced by a large variety of plants. As used herein, terpenes include terpenoids. Terpenes may be classified in various ways, such as by their sizes. For example, suitable terpenes may include monoterpenes, sesquiterpenes, or triterpenes. At least some terpenes are expected to interact with, and potentiate the activity of, cannabinoids.

Examples of terpenes known to be extractable from cannabis include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof.

Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in US Patent Application Pub. No. US2016/0250270, which is incorporated herein by reference in its entirety for all purposes.

### 4. Flavonoid

Flavonoids (or bioflavonoids) (from the Latin word flavus meaning yellow, their color in nature) are a class of plant and fungus secondary metabolites, and can be used as one or more additive in the formulations.

Chemically, flavonoids have the general structure of a 15-carbon skeleton, which consists of two phenyl rings (A and B) and heterocyclic ring (C). This carbon structure can be abbreviated C6-C3-C6. According to the IUPAC nomenclature, they can be classified into: flavonoids or bioflavonoids, isoflavonoids, derived from 3-phenylchromen-4-one (3-phenyl-1,4-benzopyrone) structure, and neoflavonoids, derived from 4-phenylcoumarine (4-phenyl-1,2-benzopyrone) structure.

The three flavonoid classes above are ah ketone-containing compounds, and as such, are anthoxanthins (flavones and flavonols). This class was the first to be termed bioflavonoids. The terms flavonoid and bioflavonoid have also been more loosely used to describe non-ketone polyhydroxy polyphenol compounds, which are more specifically termed flavanoids. The three cycle or heterocycles in the flavonoid backbone are generally called ring A, B and C. Ring A usually shows a phloroglucinol substitution pattern.

Flavonoids are widely distributed in plants, fulfilling many functions. Flavonoids are the most important plant pigments for flower coloration, producing yellow or red/blue pigmentation in petals designed to attract pollinator animals. In higher plants, flavonoids are involved in UV filtration, symbiotic nitrogen fixation and floral pigmentation. They may also act as chemical messengers, physiological regulators, and cell cycle inhibitors. Flavonoids secreted by the root of their host plant help Rhizobia in the infection stage of their symbiotic relationship with legumes like peas, beans, clover, and soy. Rhizobia living in soil can sense the flavonoids and triggers the secretion of Nod factors, which in turn are recognized by the host plant and can lead to root hair deformation and several cellular responses such as ion fluxes and the formation of a root nodule. In addition, some flavonoids have inhibitory activity against organisms that cause plant diseases, e.g. Fusarium oxysporum.

Isoflavones use the 3-phenylchromen-4-one skeleton (with no hydroxyl group substitution on carbon at position 2). Examples include: Genistein, Daidzein, Glycitein, Isoflavanes, Isoflavandiols, Isoflavenes, Coumestans, and Pterocarpans.

Exemplary flavonoids include Apigenin, beta-sitosterol, cannaflavin A, kaempferol, luteolin, orientin, and quercetin.

### 5. Cannabis Oil Extraction

Extraction in natural products chemistry is a separation process comprising the separation of a substance from a matrix of natural materials and includes liquid-liquid extraction, solid phase extraction and what is commonly referred to as super-critical extraction. The distribution of any given compound or composition between two phases is an equilibrium condition described by partition theory. This is based on exactly how the desired material moves from a first solution, typically water or other material capable of dissolving a desired material with a first solubility of the desired material, into second material, typically an organic or other immiscible layer having a second solubility of the desired material layer. Super-critical (supercritical) extraction involves entirely different phenomenon and will be described below.

There exist several types of extraction, including liquid-liquid extraction, solid-phase extraction, solid-phase microextraction, Soxhlet extraction, fizzy extraction and super-critical CO₂ (supercritical carbon dioxide) extraction.

Once various fractions of desired material have been obtained by any method such as any of fractionation and purification methods known in the art, any number of the fractions can be recombined. The recombination can be by simple mixing or by various mechanical.

### 6. Controlled Cannabis-Associated Effect

There are a number of options for designing the cannabis-infused product having a controlled onset and controlled offset of a cannabinoid profile described herein.

For example, the cannabis-infused product may include a first agent that modulates the absorption of one or more cannabinoid(s) contained in the particular cannabinoid profile. Such agent may include an encapsulating agent, a mucolytic, an efflux blocker, or any combinations thereof, which is selected to impart the controlled onset of the cannabinoid profile (e.g., a fast onset).

For example, the cannabis-infused product may include a second agent that modulates the absorption of the one or more cannabinoid(s), which is selected to impart the controlled offset of the cannabinoid profile so as to obtain an extended cannabis-associated effect. Such agent may also include an encapsulating agent, a mucolytic, an efflux blocker, or any combinations thereof, which is selected to impart the controlled offset of the cannabinoid profile.

The cannabis-infused product may thus include first and second agents, which are selected so as to obtain the controlled onset and controlled offset of the cannabinoid profile.

For example, the cannabis-infused product may include a first composition containing the first agent which is selected to impart a fast onset of the cannabis effect associated with the cannabinoid profile. The cannabis-infused product may further include a second composition containing the second agent which is selected to impart a delayed onset of the cannabinoid profile. In other words, the first and second agents can be selected such that the use of the cannabis-infused product results in a differential absorption rate of the first and second compositions—i.e., the first composition absorbing faster than the second composition, thus resulting in a faster onset associated with the cannabinoid profile and an extended cannabis-associated effect due to the later onset of the second composition which exposes the user to the cannabinoid profile over a longer period of time.

In such non-limiting embodiment, the first agent and the second agent are thus different in terms of the result obtained over the absorption rate of their respective cannabinoid profile load. Such difference in terms of the result obtained over the absorption rate can be obtained, for example, by having between the first and the second compositions, different combinations of encapsulating agents, mucolytic, or efflux blockers, or by having different proportions thereof.

For example, the first agent may form a microencapsulation composition for encapsulating a first portion of the cannabinoid profile so as to impart the herein described fast onset. In such embodiment, this microencapsulation composition may further include mucolytic, efflux blockers, or combinations thereof, if desired.

For example, the second agent may also form a microencapsulation composition but in this case, being for encapsulating a second portion of the cannabinoid profile so as to impart the herein described delayed onset. In such embodiment, this microencapsulation composition may also further include mucolytic, efflux blockers, or combinations thereof, if desired.

For example, the first and second compositions may both include an emulsion. In a non-limiting embodiment, the first and second compositions may include a respective emulsion having a specific droplet size distribution so as to impart the afore-mentioned fast onset and delayed onset.

For example, the first composition may include an emulsion having a first particle size distribution (PSD₁) which imparts the fast onset and the second composition may include an emulsion having a second particle size distribution (PSD₂) which imparts the delayed onset, where PSD₁<PSD₂.

For example, the first composition may include an emulsion having mucolytic, efflux blockers, or combinations thereof, which imparts the fast onset, and the second composition may include an emulsion having a different mucolytic, efflux blockers, or combinations thereof, which imparts the delayed onset.

In one practical implementation, the first composition may have a PSD₁ of ≤200 nm to impart the fast onset, or ≤100 nm, or ≤80 nm, or ≤70 nm, or ≤60 nm, or ≤50 nm, or ≤40 nm, or ≤30 nm, or ≤20 nm, or ≤10 nm, or any size value therein. Preferably, the first composition has a PSD₁ of from 10 nm to 80 nm, or from 10 nm to 60 nm, or from 10 to 40 nm, or any size value therein.

In one practical implementation, the second composition may have a PSD₂ of >200 nm to impart the delayed onset, or ≥300 nm, or ≥400 nm, or ≥500 nm, or ≥600 nm, or ≥700 nm, or ≥800 nm, or ≥900 nm, or >1000 nm.

That the PSD is a key factor to modulate the afore-mentioned fast onset and delayed onset is surprising and unexpected at least because there is no clear agreement in the art with respect to emulsion droplet size and permeation properties of a given molecule entrapped in said emulsion, and to the inventors knowledge, there has not been any scientific reports testing emulsion PSD effect on cannabinoid permeation through mucous or skin membranes. Indeed, to name a few cases highlighting the lack of clear agreement in the art: Izquierdo et al. (Skin pharmacology and physiology, 20. 263-70) have reported that there is no influence of emulsion droplet size on the in vitro dermal and transdermal skin penetration of tetracaine within the droplet size range studied (3 macro-emulsions with droplet sizes >1000 nm and 3 nano-emulsions with droplet sizes <100 nm); Onodera et al. (Int. J. of Mol. Med., vol. 35, Issue 6, 2015, p. 1720-1728) investigated the effects of particle diameter (50, 100 and 200 nm) on the bioactivities of curcumin lipid nanoemulsions, and found that the 100-nm emulsion had the best bioactivity both in vitro and in vivo suggesting that smaller PSD is not necessarily a guarantee of success. Odberg et al. (Eur. J. Pharm. Sci. 2003; 20(4-5): 375-382) demonstrated comparable bioavailability of cyclosporine in humans administered emulsion formulations possessing droplet sizes of 0.2 μm, 16 μm or 20 μm; Smidt et al. (Int. J. Pharm., 2004; 270(1-2): 109-118) demonstrated comparable bioavailability of penclomedine in rats administered the drug as a solution in MCT oil or as an emulsion with droplet size of either 160 nm or 710 nm; Khoo et al. (Int. J. Pharm, 1998; 167(12): 155-164)) demonstrated comparable bioavailability of halofantrine in dogs administered an emulsion with droplet size of either 119 nm or 52 nm.

### 7. Microencapsulation

Microencapsulation processes may include one or more of the emulsification and nanoemulsification techniques described below.

For example, a microencapsulation process may involve mixing, homogenization, injection, spray drying, spray cooling, spray chilling, freeze-drying, air suspension coating, fluidized-bed extrusion, centrifugal extrusion, coacervation, rotational suspension separation, cocrystallization, liposome entrapment, interfacial polymerization, molecular inclusion, microfluidization, ultrasonication, physical adsorption, complex formation, nanosized self-assembly, or any combination thereof. The microencapsulation process may be assisted or accelerated by the application of heat, e.g., through microwave irradiation. Mixing may be modelled using idealized chemical reactors, which may include, but are not limited to, batch reactors, continuous stirred-tank reactors, and plug flow reactors.

Microencapsulation compositions may include emulsions, nanoemulsions, micelles, solid lipid nanoparticles, nanostructured lipid carriers, liposomes, nanoliposomes, niosomes, polymer particles, or hydrogel particles.

In some embodiments, a cannabinoid may be solubilized in a carrier oil or solvent, and then microencapsulated in an emulsion or a nanoemulsion. Emulsions are fluid compositions in which liquid droplets are dispersed in a liquid. The droplets may be amorphous, liquid-crystalline, or any mixture thereof. The diameters of the droplets constituting the dispersed phase usually range from approximately 10 nm to 100 μm. An emulsion is termed an oil/water (O/W) emulsion if the dispersed phase is an organic material and the continuous phase is water or an aqueous solution, or termed water/oil (W/O) if the dispersed phase is water or an aqueous solution and the continuous phase is an organic liquid (an “oil”). In the context of the present disclosure, an emulsion composition is classified based on its particle radius as either a nanoemulsion (r<100 nm) or a conventional emulsion (r>100 nm).

An emulsion is a thermodynamically unfavorable system that tends to break down and revert back to its original state of two or more immiscible liquids. To form an emulsion that is (kinetically) stable for a reasonable period of time, the droplets must be prevented from merging together after they have been formed. This is typically achieved by including substances known as stabilizers that include, but are not limited to, emulsifiers, weighting agents, ripening inhibitors, or texture modifiers. Any food-grade stabilizer known for use in beverage emulsions can be employed as the food-grade emulsion stabilizer in the emulsions described herein.

Emulsifiers are surface-active molecules that adsorb to the surface of newly formed droplets during homogenization, forming a protective layer preventing aggregation. Examples of suitable emulsifiers include, but are not limited to, polysaccharide-based emulsifiers, protein-based emulsifiers, small molecule surfactants, and mixtures thereof.

Examples of suitable polysaccharide-based emulsifiers include, but are not limited to, gum arabic, modified starches such as octenyl succinate modified starches, modified cellulose such as methyl cellulose, hydroxypropyl cellulose, methyl hydroxypropyl cellulose, and carboxymethylcellulose, certain types of pectin such as beet pectin, soy soluble polysaccharide, corn fiber gum, and mixtures thereof.

Examples of suitable protein-based emulsifiers include, but are not limited to, globular proteins such as whey protein and whey protein ingredients such as whey protein concentrate, whey protein isolate, and highly purified protein fractions such as β-lactoglobulin and α-lactalbumin, flexible proteins such as gelatin and caseins such as sodium caseinate, calcium caseinate, and purified protein fractions, such as β-casein. Milk-derived proteins (e.g., caseins, in either monomeric or micellar form, or whey proteins) may be used to form dairy emulsions. Milk proteins function as surface active ingredients in emulsions because of their amphiphilic structure, and they contribute to the stability of the emulsion droplets by a combination of electrostatic and steric stabilization mechanisms.

Examples of small molecule surfactants include, but are not limited to, Tweens™ (polysorbates) such as Tween 20 (polyoxyethylene sorbitan monolaurate), Tween 40 (polyoxyethylene sorbitan monopalmitate), Tween 60 (polyoxyethylene sorbitan monostearate), and Tween 80 (polyoxyethylene sorbitan monooleate), sugar esters such as sucrose monopalmitate, sucrose monostearate, sucrose distearate, sucrose polystearate, quillaja saponin (Q-Naturale™) and components thereof, sorbitan esters (Spans™) such as Span 20 (sorbitan monolaurate), Span 40 (sorbitan monopalmitate), Span 60 (sorbitan monostearate), Span 80 (sorbitan monooleate).

Emulsifiers such as lecithin, gum arabic, and octenyl succinate starches produce an emulsion with a negative charge on the surface of the droplet, which attracts pro-oxidant metal ions. This can be overcome using proteins, typically those derived from milk or soya.

An emulsion or nanoemulsion microencapsulation composition may be formed using any of the techniques available to fabricate emulsions and nanoemulsions. The techniques available are commonly classified as either high or low energy approaches.

High energy approaches use mechanical devices known as “homogenizers” that generate intense disruptive forces that mix the oil and water phases together, as well as break larger droplets into smaller ones. O/W emulsions are usually prepared by homogenizing an oil phase and a watery phase together in the presence of a water-soluble hydrophilic emulsifier. A variety of specialized homogenization equipment is available for fabricating emulsions and nanoemulsions that include, but are not limited to, high shear mixers, high pressure valve homogenizers, microfluidizers, colloid mills, ultrasonic homogenizers, and membrane and microchannel homogenizers.

High shear mixers are a type of rotor-stator device that homogenizes oil, water, and other ingredients in a batch process. Typically, the droplets produced by a high shear mixer range between about 1 and 10 μm in diameter. A suitable vessel may contain as a few cm³ or as large as several m³. The rapid rotation of the mixing head generates a combination of longitudinal, rotational, and radial velocity gradients in the fluids, which disrupts the interfaces between the oil and water phases, causing the liquids to become intermingled, and breaks the larger droplets into smaller ones. Efficient homogenization is achieved when the horizontal and vertical flow profiles distribute the liquids evenly throughout the vessel, which can be facilitated by having baffles fixed to the inside walls of the vessel. The design of the mixing head determines the efficiency of the homogenization process, and a number of different types are available for different situations, for example, blades, propellers, and turbines.

High-pressure valve homogenizers are used to produce fine emulsions from pre-existing emulsions (“coarse emulsion”), with emulsion droplets as small as 0.1 μm. The homogenizer has a pump that pulls the coarse emulsion into a chamber on its backstroke and then forces it through a narrow valve at the end of the chamber and on its forwards stroke it experiences a combination of intense disruptive forces that cause the larger droplets to be broken down to smaller ones. The flow regime that is responsible for disrupting the droplets in a particular high pressure valve homogenizer depends on the characteristics of the material being homogenized, the size of the homogenizer, and the design of the homogenization nozzle.

Microfluidization creates emulsions with very fine droplets whose diameter can be less than 0.1 μm. This type of homogenizer typically consists of a fluid inlet (single or double), some kind of pumping device, and an interaction chamber containing two channels. Fluids are introduced into the homogenizer, accelerated to a high velocity and then made to simultaneously impinge with each other on a solid surface, which causes the fluids to intermingle and disrupt larger droplets.

Colloid mills are used to homogenize medium and high viscosity liquids. A colloid mill typically contains two disks: a rotor (a rotating disk) and a stator (a static disk). The liquids and other ingredients to be homogenized are usually fed into the center of the colloid mill in the form of a pre-existing emulsion. The intensity of the shear stresses (and therefore the droplet disruption forces) can be altered by varying the rotation speed, gap thickness, rotor/stator type, and throughput to reduce droplet sizes. Typically, colloid mills can be used to produce emulsions with droplet diameters around 1 and 5 μm.

Ultrasonic homogenizers use high-intensity ultrasonic waves that generate intense shear and pressure gradients within a material that disrupt droplets mainly through cavitation and turbulent effects. The present invention can use any of the available methods that are available for generating high-intensity ultrasonic waves including, but not limited to, piezoelectric transducers and liquid jet generators.

Membrane homogenizers can be used in two main ways to process emulsions, direct homogenization and premix homogenization. Direct homogenization involves forming an emulsion directly from the separate oil and water phases in the presence of a suitable emulsifier. Premix homogenization involves reducing the size of the droplets present within an existing coarse emulsion. The droplet size attained depends on the membrane pore size, the oil-water interfacial tension, the applied pressure, the flow profile of the continuous phase, and the type and amount of emulsifier used.

Low energy approaches to produce emulsions and nanoemulsions rely on the spontaneous formation of oil droplets in surfactant-oil-water mixtures which either their composition or environment is altered in a controlled way. Examples of low energy methods include, but are not limited to, spontaneous emulsification methods, emulsion inversion point methods, and phase inversion temperature methods.

Spontaneous emulsification involves titrating a mixture of oil and water-soluble surfactant into a water phase with continuous stirring. Small oil droplets are spontaneously formed at the oil-water boundary as the surfactant molecules move from the oil phase to the water phase. The spontaneous emulsification method has been used widely within the pharmaceutical industry to encapsulate and deliver lipophilic drugs. Such systems are known as either self-emulsifying drug delivery systems (SEDDS) or self-nanoemulsifying drug delivery systems (SNEDDS) depending on the droplet size produced. Self-emulsifying formulations are readily dispersed in the gastrointestinal tract, where the motility of the stomach and small intestine provides the agitation necessary for emulsification.

Emulsion inversion point methods involve titrating water into a mixture of oil and water-soluble surfactant with continuous stirring. As increasing amounts of water are added, a W/O emulsion is initially formed, then an O/W/O emulsion, and then an O/W emulsion.

Phase inversion temperature (PIT) methods rely on heating a surfactant-oil-water mixture around or slightly above its PIT and the quench cooling with continuous stirring. When the emulsion passes through the PIT, the optimum curvature tends towards unity, thereby leading to an ultralow interfacial tension and a highly dynamic interface. For a general overview of emulsification technology, see, e.g., McClements, David J., Food Emulsions: Principles, Practices, and Techniques, 3^{rd} ed (Boca Raton, Fla. CRC Press, 2016).

In some embodiments, a cannabinoid may be microencapsulated in micelles. Micelles consist of small clusters of surfactant molecules that self-assemble into a structure where the hydrophobic tails are located in the interior and the hydrophilic heads are located at the exterior. Micelles are thermodynamically stable systems under a particular range of compositional and environmental conditions, and should therefore form spontaneously. Nevertheless, some form of energy often has to be applied during their formation (such as simple mixing) to overcome kinetic energy barriers to the self-assembly of the surfactant molecules. Micelles are one of the smallest colloidal particles that are widely used as delivery systems, with diameters typically in the range from about 5 to 20 nm. Nonpolar active agents can be solubilized within the hydrophobic interior of micelles, whereas amphiphilic active agents can be incorporated at their exterior, with the loading capacity depending on the molecular dimensions of the active agents and the optimum curvature of the surfactant monolayer. Larger thermodynamically stable micelles (e.g., diameters up to 100 nm) may also contain an oil phase and possibly a co-surfactant. Termed “microemulsions” by IUPAC, larger thermodynamically stable micelles can solubilize higher levels of nonpolar active agents. They are usually fabricated from one or more small-molecule surfactants, but amphiphilic block copolymers can also be used.

In some embodiments, a cannabinoid may be microencapsulated in solid lipid nanoparticles or nanostructured lipid carriers. Solid lipid nanoparticles (SLNs) have similar structures to nanoemulsions (or emulsions), but the oil phase is crystallized rather than liquid. SLNs are typically fabricated by preparing an oil-in-water nanoemulsion at a temperature above the melting point (Tₘ) of the oil phase, and then cooling the composition well below Tₘ to promote droplet crystallization. In principle, the crystallization of the lipid phase slows down molecular diffusion processes inside the particles, which may help to protect an encapsulated active agent from chemical degradation. SLNs have proven to be useful delivery systems for many applications in the pharmaceutical industry, where they are mainly used to encapsulate hydrophobic drugs. However, if the lipid phase is not carefully selected there can be appreciable challenges to their utilization for this purpose. Lipids that form highly regular crystalline structures (such as pure triacylglycerols) have a tendency to expel other nonpolar substances when they undergo a liquid-to-solid transition. Moreover, there may be an appreciable change in the morphology of the lipid nanoparticles, from spherical to irregular, when the lipid phase crystallizes or undergoes a polymorphic transition. As a result of the increase in particle surface area, there may be insufficient emulsifier to coat the particles, which leads to extensive aggregation. These problems can be overcome by using nanostructured lipid carriers (NLCs). In this case, a lipid phase is selected that forms more irregular crystals when it solidifies, which leads to less expulsion of encapsulated active agents and less particle aggregation.

In some embodiments, a cannabinoid may be microencapsulated in liposomes, nanoliposomes, or niosomes. Liposomes (diameter >100 nm) and nanoliposomes (diameter <100 nm) are colloidal compositions that are composed of particles made up of concentric layers of phospholipid bilayers. Niosomes are formed when non-ionic surfactants assemble into similar structures. The bilayers form due to the hydrophobic effect, that is, the tendency for the composition to reduce the contact area between the nonpolar phospholipid or surfactant tails and water. These compositions may contain one (unilamellar) or numerous (multilamellar) phospholipid bilayers depending on the preparation method and ingredients used. Hydrophilic functional ingredients can be trapped inside the aqueous interior of liposomes and nanoliposomes, whereas amphiphilic and lipophilic active agents can be trapped in the bilayer region. Liposomes and nanoliposomes can be fabricated from natural components, such as phospholipids. Cholesterol is often added to the formulation as it increases rigidity strength of the membrane and confers steric stability. Egg yolk- and soy-derived phosphatidylcholines are commonly used to form liposomes, whereas Tween™ 80, Span™ 80 and sucrose laurate have been used to form niosomes.

In some embodiments, a cannabinoid may be microencapsulated in polymer or hydrogel particles. Polymer microparticles (diameter >100 nm) and nanoparticles (diameter <100 nm) are fabricated from either synthetic or natural polymers, such as proteins and polysaccharides. Commonly, they are produced from antisolvent precipitation methods where a polymer dissolved in a good solvent is injected into a poor solvent, which promotes spontaneous particle formation. Hydrogel particles (sometimes called nanogels or microgels) may also be fabricated from synthetic or natural polymers, but they contain higher levels of water (typically >80% to 90%). A wide variety of different methods are available for producing hydrogel particles including injection, templating, emulsion, and phase separation methods. The composition and porosity of hydrogel particles must be carefully controlled to ensure appropriate loading, retention, and release properties.

In some embodiments, once a stable encapsulation composition has been produced, the encapsulation composition can dehydrated to form a powder, typically using spray drying. For example, the emulsion may be dried to obtain a water activity (a_{w}) of less than 0.75, for example 0.04≤a_{w}≤0.75, or example 0.04≤a_{w}≤0.3. Water activity may be measured using an Aqualab Water Activity Meter 4TE (Decagon Devices, Inc., U.S.A.). For extra protection, the resulting powder can be atomized and coated with a secondary layer, typically a high melting fat or starch. Alternative methods of preparing the dried powder include, but are not limited to, pan coating, air-suspension coating, centrifugal extrusion, vibrational nozzle technique, freeze-drying or using a food dehydrator. For extra protection, the resulting powder can be atomized and coated with a secondary layer, typically a high melting fat or starch. The powder composition can be used for use in beverages and foods. This is also applicable to the above described emulsion, which may also be dried using any method as known in the drying arts to evaporate the water phase of the emulsion, and possibly none, some or essentially all of the carrier solvent. For example, in one embodiment, the emulsions are spray dried to form the powder formulation.

In some embodiments, the powder can be diluted with a bulking agent or a mixture of bulking agents. Suitable bulking agents include, for example, gum arabic, waxy maize starch, dextrin, maltodextrin, polydextrose, inulin, fructooligo saccharide, sucrose, glucose, fructose, galactose, lactose, maltose, trehalose, cellobiose, lactulose, ribose, arabinose, xylose, lyxose, allose, altrose, mannose, gulose, talose, erythritol, threitol, arabitol, xylitol, mannitol, ribitol, galactitol, fucitol, inositol, maltitol, sorbitol, isomalt, lactitol, polyglycitol, iditol, volemitol, maltotriitol, maltotetraitol, maltol, stevia, stevioside, rebaudioside, neotame, sucralose, saccharin, sodium cyclamate, aspartame, acesulfame potassium, chitin, and chitosan.

In some aspects, the bulking material may comprise a sweetener, pH modifier, pH stabilizer, antimicrobial preservative, antioxidant, texture modifier, colorant or combinations thereof.

In some embodiments, the herein described emulsion of cannabinoids may include, for example, per total volume of emulsion up to 1 g/ml, up to 750 mg/ml, up to 700 mg/ml, up to 650 mg/ml, up to 600 mg/ml, up to 550 mg/ml, up to 500 mg/ml, up to 450 mg/ml, up to 400 mg/ml, up to 350 mg/ml, up to 300 mg/ml, up to 250 mg/ml, up to 200 mg/ml, up to 150 mg/ml, up to 100 mg/ml, up to 50 mg/ml, up to 40 mg/ml, up to 35 mg/ml, up to 30 mg/ml, up to 25 mg/ml, up to 20 mg/ml, or up to 15 mg/ml of a specific cannabis extract such as THC, CBD, terpene (e.g., D-limonene) or any mixtures thereof, and the like.

### 8. Methods of Manufacturing Nanoemulsions

There are many options to obtain the herein described nanoemulsions.

In one option, a cannabis oil extract is mixed with water in presence of a suitable amount of one or more emulsifiers, and the mixture is then subjected to a shear mixer so as to obtain an emulsion having a desired particle size distribution (PSD). In some example, the shear mixer may be a high or low shear mixer depending on the specifics on the application. The low-shear mixer may be a rotor-stator mixer. The high shear mixer may be a microfluidizer. The mixture may be passed through each mixer one or more times. Pressure, number of passes, and temperature of the process may be adjusted.

In another option, a cannabis oil extract is gently warmed (e.g., in a water bath) and is mixed with a starch-based powder, such as maltodextrin, to create a uniform concentrated cannabis extract powder. This powder is then dissolved in hot water to dissolve the powder and emulsify the extract, as disclosed for example in U.S. Pat. No. 9,629,886 B2, which is incorporated herein by reference in its entirety for all purposes. Other types of powders fit for human consumption may be used in place of the starch-based powder, including but not limited to, whey protein isolate (both dairy-based and plant-based), xanthan gum, guar gum (guaran), mono-and diglycerides, and carboxymethylcellulose (cellulose gum) so long as they absorb the oil when blended together, dissolve when added to a liquid, remain dissolved in that liquid and have no post-mixing separation of the powder and the oil.

In yet another option, a cannabis oil extract is mixed with a heated carrier oil. This mixture is then mixed with an aqueous solution in presence of one or more emulsifying compound, as disclosed for example in WO 2017/180948.

In yet another option, a cannabis oil extract is mixed with a carrier oil, such as olive oil or coconut oil (MCT) or any other suitable oil. This mixture is then mixed with one or more emulsifier and sonicated to obtain an oil-cannabis mixture. The sonication step may be performed using an ultrasonic homogenizer. This mixture can then be emulsified by adding an amount of water and obtain a desired PSD, for example a nanoemulsion with droplet sizes of about 20 to 40 nm.

In yet another option, a cannabis oil extract is mixed with a carrier oil and a first emulsifier to obtain a first mixture. This mixture is heated up to 110° C. and cooled down for an appropriate period of time, e.g., for 24 hours. Water is mixed with a second emulsifier and heated up to 45° C., cooled down for an appropriate period of time, e.g., for 24 hours, to obtain a second mixture. The first and the second mixtures are then mixed at room temperature and sonicated to obtain an emulsion having a desired PSD. For example, the oil volume fraction can be at φₒ=0.10 and the total emulsifier volume fraction can be at φₛ=0.08. For example, the sonication time can be between 5 and 7.5 minutes. For example, the use of Tween™ 85 and Span™ 85 at 10 wt % as emulsifiers can produce particles ranging in diameter from 84 nm to 122 nm.

In yet another option, a water-soluble surfactant is mixed with water to form an aqueous phase, which is then heated to 70° C., An oil-soluble surfactant and a cannabis oil extract are mixed to form an oil phase, which is then heated to 70° C. The aqueous phase is then added drop-by-drop to the oil phase, and the resulting mixture is stirred at a constant rate for 30 minutes at a temperature of 70° C. The use of a combination of Tween 80 and Span 80 at 5 wt % as emulsifiers can produce particles ranging in diameter from about 500 nm to about 1050 nm.

In yet another option, water and a lipid source are mixed and heated to boiling to obtain a boiling aqueous composition. Cannabis material is then enclosed in a tea bag (or similar porous enclosure) and steeped in the boiling aqueous composition to diffuse cannabis oil extract into the aqueous composition and obtain an emulsion. The lipid source may include, but is not limited to, milk such as 10% milk, or butter, or combinations thereof. The ratio of the water to the lipid source may be about 4:1. The cannabis material may be the bud or the trim. The cannabis material may be processed using a hand miller, such as a handheld food processor, or an industrial miller. The heating step may be performed using an electric water heater or a microwave (e.g., set to a length of time of 2 minutes). The steeping step may last from about 3 minutes to about 10 minutes.

In some embodiments, procedures may be employed during manufacturing of the emulsions (or thereafter) to ensure that the cannabis-infused products are not contaminated with bacteria, yeast, or mold.

For example, the emulsion may be processed and/or made such that there is less than 100,000 CFU of total viable aerobic bacteria count; less than 100,000 CFU/g of total yeast and mold count, preferably less than 10,000 CFU/g, less than 1000 CFU of bile-tolerant gram negative bacteria; less than 1000 CFU/g of total coliforms count, preferably less than 100 CFU/g, or any combinations thereof.

It will be readily apparent to the person of skill how to implement such procedures using known techniques in the art and as such, and for conciseness sake, will not be further discussed here.

In some embodiments, the herein described procedures afford a cannabis-infused product which incorporates the cannabinoid profile in a stable manner. In other words, the cannabis-infused product advantageously remains stable in that there is close to no deterioration of the product appearance within the expected storage shelf-life.

In some embodiments, a cannabis-infused product provided herein may be stable for at least about 1 month at 4° C. In some embodiments, the cannabis-infused product provided herein may be stable for at least about 2 months at 4° C. In some embodiments, the cannabis-infused product provided herein may be stable for at least about 3 months at 4° C. In some embodiments, the cannabis-infused product provided herein may be stable for at least about 4 months at 4° C. In some embodiments, the cannabis-infused product provided herein may be stable for at least about 5 months at 4° C. In some embodiments, the cannabis-infused product provided herein may be stable for at least about 6 months at 4° C. In some embodiments, the cannabis-infused product provided herein may be stable for at least about 7 months at 4° C. In some embodiments, the cannabis-infused product provided herein may be stable for at least about 8 months at 4° C. In some embodiments, the cannabis-infused product provided herein may be stable for at least about 9 months at 4° C. In some embodiments, the cannabis-infused product provided herein may be stable for at least about 10 months at 4° C. In some embodiments, the cannabis-infused product provided herein may be stable for at least about 11 months at 4° C. In some embodiments, the cannabis-infused product provided herein may be stable for at least about 1 year at 4° C.

In some embodiments, a cannabis-infused product provided herein may be stable for at least about 1 month at room temperature. In some embodiments, a cannabis-infused product provided herein may be stable for at least about 2 months at room temperature. In some embodiments, a cannabis-infused product provided herein may be stable for at least about 3 months at room temperature. In some embodiments, a cannabis-infused product provided herein may be stable for at least about 4 months at room temperature. In some embodiments, a cannabis-infused product provided herein may be stable for at least about 5 months at room temperature. In some embodiments, a cannabis-infused product provided herein may be stable for at least about 6 months at room temperature. In some embodiments, a cannabis-infused product provided herein may be stable for at least about 7 months at room temperature. In some embodiments, a cannabis-infused product provided herein may be stable for at least about 8 months at room temperature. In some embodiments, a cannabis-infused product provided herein may be stable for at least about 9 months at room temperature. In some embodiments, a cannabis-infused product provided herein may be stable for at least about 10 months at room temperature. In some embodiments, a cannabis-infused product provided herein may be stable for at least about 11 months at room temperature. In some embodiments, a cannabis-infused product provided herein may be stable for at least about 1 year at room temperature.

### 9. Precursor Compositions

There are many combinations possible for designing a precursor composition for infusing (used interchangeably here with blending, diluting, and the like) a product base so as to obtain the herein described cannabis-infused product. The following section provides a number of examples of such precursor compositions.

It will be apparent to the reader that while the following precursor compositions are designed to impart a fast onset for THC and a delayed onset of THC when the precursor composition is infused into a product base so as to obtain the herein described cannabis-infused product, the reader will understand that these examples can be applied to any other cannabinoid profile.

In some embodiments, the fast onset microencapsulation composition of THC comprises particles having an average size of less than about 100 nm, and the delayed onset microencapsulation composition of THC comprises particles having an average size of more than about 100 nm. As used herein, the average size of the particles refers to the average diameter of the particles.

In some embodiments, the fast onset microencapsulation composition of THC comprises particles having an average size of less than about 100 nm, and the delayed onset microencapsulation composition of THC comprises particles having an average size of more than about 150 nm.

In some embodiments, the fast onset microencapsulation composition of THC comprises particles having an average size of less than about 100 nm, and the delayed onset microencapsulation composition of THC comprises particles having an average size of more than about 200 nm.

In some embodiments, the fast onset microencapsulation composition of THC comprises particles having an average size of less than about 100 nm, and the delayed onset microencapsulation composition of THC comprises particles having an average size of more than about 250 nm.

In some embodiments, the fast onset microencapsulation composition of THC comprises particles having an average size of less than about 100 nm, and the delayed onset microencapsulation composition of THC comprises particles having an average size of more than about 300 nm.

In some embodiments, the fast onset microencapsulation composition of THC comprises particles having an average size of less than about 100 nm, and the delayed onset microencapsulation composition of THC comprises particles having an average size of more than about 350 nm.

In some embodiments, the fast onset microencapsulation composition of THC comprises particles having an average size of less than about 100 nm, and the delayed onset microencapsulation composition of THC comprises particles having an average size of more than about 400 nm.

In some embodiments, the fast onset microencapsulation composition of THC comprises particles having an average size of less than about 100 nm, and the delayed onset microencapsulation composition of THC comprises particles having an average size of more than about 450 nm.

In some embodiments, the fast onset microencapsulation composition of THC comprises particles having an average size of less than about 100 nm, and the delayed onset microencapsulation composition of THC comprises particles having an average size of more than about 500 nm.

In some embodiments, the fast onset microencapsulation composition of THC comprises particles having an average size of less than about 100 nm, and the delayed onset microencapsulation composition of THC comprises particles having an average size of more than about 600 nm.

In some embodiments, the fast onset microencapsulation composition of THC comprises particles having an average size of less than about 100 nm, and the delayed onset microencapsulation composition of THC comprises particles having an average size of more than about 700 nm.

In some embodiments, the fast onset microencapsulation composition of THC comprises particles having an average size of less than about 100 nm, and the delayed onset microencapsulation composition of THC comprises particles having an average size of more than about 800 nm.

In some embodiments, the fast onset microencapsulation composition of THC comprises particles having an average size of less than about 100 nm, and the delayed onset microencapsulation composition of THC comprises particles having an average size of more than about 900 nm.

In some embodiments, the fast onset microencapsulation composition of THC comprises particles having an average size of less than about 100 nm, and the delayed onset microencapsulation composition of THC comprises particles having an average size of more than about 1 μm.

In some embodiments, the fast onset microencapsulation composition of THC comprises particles having an average size of less than about 100 nm, and the delayed onset microencapsulation composition of THC comprises particles having an average size of more than about 2 μm.

In some embodiments, the fast onset microencapsulation composition of THC comprises particles having an average size of less than about 100 nm, and the delayed onset microencapsulation composition of THC comprises particles having an average size of more than about 3 μm.

In some embodiments, the fast onset microencapsulation composition of THC comprises particles having an average size of less than about 100 nm, and the delayed onset microencapsulation composition of THC comprises particles having an average size of more than about 4 μm.

In some embodiments, the fast onset microencapsulation composition of THC comprises particles having an average size of less than about 100 nm, and the delayed onset microencapsulation composition of THC comprises particles having an average size of more than about 5 μm.

In some embodiments, the fast onset microencapsulation composition of THC comprises particles having an average size of less than about 100 nm, and the delayed onset microencapsulation composition of THC comprises particles having an average size of more than about 6 μm.

In some embodiments, the fast onset microencapsulation composition of THC comprises particles having an average size of less than about 100 nm, and the delayed onset microencapsulation composition of THC comprises particles having an average size of more than about 7 μm.

In some embodiments, the fast onset microencapsulation composition of THC comprises particles having an average size of less than about 100 nm, and the delayed onset microencapsulation composition of THC comprises particles having an average size of more than about 8 μm.

In some embodiments, the fast onset microencapsulation composition of THC comprises particles having an average size of less than about 100 nm, and the delayed onset microencapsulation composition of THC comprises particles having an average size of more than about 9 μm.

In some embodiments, the fast onset microencapsulation composition of THC comprises particles having an average size of less than about 100 nm, and the delayed onset microencapsulation composition of THC comprises particles having an average size of more than about 10 μm.

In some embodiments, the fast onset microencapsulation composition of THC comprises particles having an average size of less than about 90 nm, and the delayed onset microencapsulation composition of TIC comprises particles having an average size of more than about 100 nm.

In some embodiments, the fast onset microencapsulation composition of THC comprises particles having an average size of less than about 90 nm, and the delayed onset microencapsulation composition of THC comprises particles having an average size of more than about 150 nm.

In some embodiments, the fast onset microencapsulation composition of THC comprises particles having an average size of less than about 90 nm, and the delayed onset microencapsulation composition of THC comprises particles having an average size of more than about 200 nm.

In some embodiments, the fast onset microencapsulation composition of THC comprises particles having an average size of less than about 90 nm, and the delayed onset microencapsulation composition of THC comprises particles having an average size of more than about 250 nm.

In some embodiments, the fast onset microencapsulation composition of THC comprises particles having an average size of less than about 90 nm, and the delayed onset microencapsulation composition of THC comprises particles having an average size of more than about 300 nm.

In some embodiments, the fast onset microencapsulation composition of THC comprises particles having an average size of less than about 90 nm, and the delayed onset microencapsulation composition of THC comprises particles having an average size of more than about 350 nm.

In some embodiments, the fast onset microencapsulation composition of THC comprises particles having an average size of less than about 90 nm, and the delayed onset microencapsulation composition of THC comprises particles having an average size of more than about 400 nm.

In some embodiments, the fast onset microencapsulation composition of THC comprises particles having an average size of less than about 90 nm, and the delayed onset microencapsulation composition of THC comprises particles having an average size of more than about 450 nm.

In some embodiments, the fast onset microencapsulation composition of THC comprises particles having an average size of less than about 90 nm, and the delayed onset microencapsulation composition of THC comprises particles having an average size of more than about 500 nm.

In some embodiments, the fast onset microencapsulation composition of THC comprises particles having an average size of less than about 90 nm, and the delayed onset microencapsulation composition of THC comprises particles having an average size of more than about 600 nm.

In some embodiments, the fast onset microencapsulation composition of THC comprises particles having an average size of less than about 90 nm, and the delayed onset microencapsulation composition of THC comprises particles having an average size of more than about 700 nm.

In some embodiments, the fast onset microencapsulation composition of THC comprises particles having an average size of less than about 90 nm, and the delayed onset microencapsulation composition of THC comprises particles having an average size of more than about 800 nm.

In some embodiments, the fast onset microencapsulation composition of THC comprises particles having an average size of less than about 90 nm, and the delayed onset microencapsulation composition of THC comprises particles having an average size of more than about 900 nm.

In some embodiments, the fast onset microencapsulation composition of THC comprises particles having an average size of less than about 90 nm, and the delayed onset microencapsulation composition of TIC comprises particles having an average size of more than about 1 μm.

In some embodiments, the fast onset microencapsulation composition of THC comprises particles having an average size of less than about 90 nm, and the delayed onset microencapsulation composition of THC comprises particles having an average size of more than about 2 μm.

In some embodiments, the fast onset microencapsulation composition of THC comprises particles having an average size of less than about 90 nm, and the delayed onset microencapsulation composition of THC comprises particles having an average size of more than about 3 μm.

In some embodiments, the fast onset microencapsulation composition of THC comprises particles having an average size of less than about 90 nm, and the delayed onset microencapsulation composition of THC comprises particles having an average size of more than about 4 μm.

In some embodiments, the fast onset microencapsulation composition of THC comprises particles having an average size of less than about 90 nm, and the delayed onset microencapsulation composition of THC comprises particles having an average size of more than about 5 μm.

In some embodiments, the fast onset microencapsulation composition of THC comprises particles having an average size of less than about 90 nm, and the delayed onset microencapsulation composition of THC comprises particles having an average size of more than about 6 μm.

In some embodiments, the fast onset microencapsulation composition of THC comprises particles having an average size of less than about 90 nm, and the delayed onset microencapsulation composition of THC comprises particles having an average size of more than about 7 μm.

In some embodiments, the fast onset microencapsulation composition of THC comprises particles having an average size of less than about 90 nm, and the delayed onset microencapsulation composition of TIC comprises particles having an average size of more than about 8 μm.

In some embodiments, the fast onset microencapsulation composition of THC comprises particles having an average size of less than about 90 nm, and the delayed onset microencapsulation composition of THC comprises particles having an average size of more than about 9 μm.

In some embodiments, the fast onset microencapsulation composition of THC comprises particles having an average size of less than about 90 nm, and the delayed onset microencapsulation composition of THC comprises particles having an average size of more than about 10 μm.

In some embodiments, the fast onset microencapsulation composition of THC comprises particles having an average size of less than about 80 nm, and the delayed onset microencapsulation composition of THC comprises particles having an average size of more than about 100 nm.

In some embodiments, the fast onset microencapsulation composition of THC comprises particles having an average size of less than about 80 nm, and the delayed onset microencapsulation composition of THC comprises particles having an average size of more than about 150 nm.

In some embodiments, the fast onset microencapsulation composition of THC comprises particles having an average size of less than about 80 nm, and the delayed onset microencapsulation composition of THC comprises particles having an average size of more than about 200 nm.

In some embodiments, the fast onset microencapsulation composition of THC comprises particles having an average size of less than about 80 nm, and the delayed onset microencapsulation composition of THC comprises particles having an average size of more than about 250 nm.

In some embodiments, the fast onset microencapsulation composition of THC comprises particles having an average size of less than about 80 nm, and the delayed onset microencapsulation composition of TIC comprises particles having an average size of more than about 300 nm.

In some embodiments, the fast onset microencapsulation composition of THC comprises particles having an average size of less than about 80 nm, and the delayed onset microencapsulation composition of THC comprises particles having an average size of more than about 350 nm.

In some embodiments, the fast onset microencapsulation composition of THC comprises particles having an average size of less than about 80 nm, and the delayed onset microencapsulation composition of THC comprises particles having an average size of more than about 400 nm.

In some embodiments, the fast onset microencapsulation composition of THC comprises particles having an average size of less than about 80 nm, and the delayed onset microencapsulation composition of THC comprises particles having an average size of more than about 450 nm.

In some embodiments, the fast onset microencapsulation composition of THC comprises particles having an average size of less than about 80 nm, and the delayed onset microencapsulation composition of THC comprises particles having an average size of more than about 500 nm.

In some embodiments, the fast onset microencapsulation composition of THC comprises particles having an average size of less than about 80 nm, and the delayed onset microencapsulation composition of THC comprises particles having an average size of more than about 600 nm.

In some embodiments, the fast onset microencapsulation composition of THC comprises particles having an average size of less than about 80 nm, and the delayed onset microencapsulation composition of THC comprises particles having an average size of more than about 700 nm.

In some embodiments, the fast onset microencapsulation composition of THC comprises particles having an average size of less than about 80 nm, and the delayed onset microencapsulation composition of THC comprises particles having an average size of more than about 800 nm.

In some embodiments, the fast onset microencapsulation composition of THC comprises particles having an average size of less than about 80 nm, and the delayed onset microencapsulation composition of THC comprises particles having an average size of more than about 900 nm.

In some embodiments, the fast onset microencapsulation composition of THC comprises particles having an average size of less than about 80 nm, and the delayed onset microencapsulation composition of THC comprises particles having an average size of more than about 1 μm.

In some embodiments, the fast onset microencapsulation composition of THC comprises particles having an average size of less than about 80 nm, and the delayed onset microencapsulation composition of THC comprises particles having an average size of more than about 2 μm.

In some embodiments, the fast onset microencapsulation composition of THC comprises particles having an average size of less than about 80 nm, and the delayed onset microencapsulation composition of THC comprises particles having an average size of more than about 3 μm.

In some embodiments, the fast onset microencapsulation composition of THC comprises particles having an average size of less than about 80 nm, and the delayed onset microencapsulation composition of THC comprises particles having an average size of more than about 4 μm.

In some embodiments, the fast onset microencapsulation composition of THC comprises particles having an average size of less than about 80 nm, and the delayed onset microencapsulation composition of THC comprises particles having an average size of more than about 5 μm.

In some embodiments, the fast onset microencapsulation composition of THC comprises particles having an average size of less than about 80 nm, and the delayed onset microencapsulation composition of THC comprises particles having an average size of more than about 6 μm.

In some embodiments, the fast onset microencapsulation composition of THC comprises particles having an average size of less than about 80 nm, and the delayed onset microencapsulation composition of THC comprises particles having an average size of more than about 7 μm.

In some embodiments, the fast onset microencapsulation composition of THC comprises particles having an average size of less than about 80 nm, and the delayed onset microencapsulation composition of THC comprises particles having an average size of more than about 8 μm.

In some embodiments, the fast onset microencapsulation composition of THC comprises particles having an average size of less than about 80 nm, and the delayed onset microencapsulation composition of THC comprises particles having an average size of more than about 9 μm.

In some embodiments, the fast onset microencapsulation composition of THC comprises particles having an average size of less than about 80 nm, and the delayed onset microencapsulation composition of THC comprises particles having an average size of more than about 10 μm.

In some embodiments, the fast onset microencapsulation composition of THC comprises particles having an average size of less than about 70 nm, and the delayed onset microencapsulation composition of THC comprises particles having an average size of more than about 100 nm.

In some embodiments, the fast onset microencapsulation composition of THC comprises particles having an average size of less than about 70 nm, and the delayed onset microencapsulation composition of THC comprises particles having an average size of more than about 150 nm.

In some embodiments, the fast onset microencapsulation composition of THC comprises particles having an average size of less than about 70 nm, and the delayed onset microencapsulation composition of THC comprises particles having an average size of more than about 200 nm.

In some embodiments, the fast onset microencapsulation composition of THC comprises particles having an average size of less than about 70 nm, and the delayed onset microencapsulation composition of THC comprises particles having an average size of more than about 250 nm.

In some embodiments, the fast onset microencapsulation composition of THC comprises particles having an average size of less than about 70 nm, and the delayed onset microencapsulation composition of THC comprises particles having an average size of more than about 300 nm.

In some embodiments, the fast onset microencapsulation composition of THC comprises particles having an average size of less than about 70 nm, and the delayed onset microencapsulation composition of THC comprises particles having an average size of more than about 350 nm.

In some embodiments, the fast onset microencapsulation composition of THC comprises particles having an average size of less than about 70 nm, and the delayed onset microencapsulation composition of THC comprises particles having an average size of more than about 400 nm.

In some embodiments, the fast onset microencapsulation composition of THC comprises particles having an average size of less than about 70 nm, and the delayed onset microencapsulation composition of THC comprises particles having an average size of more than about 450 nm.

In some embodiments, the fast onset microencapsulation composition of THC comprises particles having an average size of less than about 70 nm, and the delayed onset microencapsulation composition of THC comprises particles having an average size of more than about 500 nm.

In some embodiments, the fast onset microencapsulation composition of THC comprises particles having an average size of less than about 70 nm, and the delayed onset microencapsulation composition of THC comprises particles having an average size of more than about 600 nm.

In some embodiments, the fast onset microencapsulation composition of THC comprises particles having an average size of less than about 70 nm, and the delayed onset microencapsulation composition of THC comprises particles having an average size of more than about 700 nm.

In some embodiments, the fast onset microencapsulation composition of THC comprises particles having an average size of less than about 70 nm, and the delayed onset microencapsulation composition of THC comprises particles having an average size of more than about 800 nm.

In some embodiments, the fast onset microencapsulation composition of THC comprises particles having an average size of less than about 70 nm, and the delayed onset microencapsulation composition of THC comprises particles having an average size of more than about 900 nm.

In some embodiments, the fast onset microencapsulation composition of THC comprises particles having an average size of less than about 70 nm, and the delayed onset microencapsulation composition of THC comprises particles having an average size of more than about 1 μm.

In some embodiments, the fast onset microencapsulation composition of THC comprises particles having an average size of less than about 70 nm, and the delayed onset microencapsulation composition of THC comprises particles having an average size of more than about 2 μm.

In some embodiments, the fast onset microencapsulation composition of THC comprises particles having an average size of less than about 70 nm, and the delayed onset microencapsulation composition of THC comprises particles having an average size of more than about 3 μm.

In some embodiments, the fast onset microencapsulation composition of THC comprises particles having an average size of less than about 70 nm, and the delayed onset microencapsulation composition of TIC comprises particles having an average size of more than about 4 μm.

In some embodiments, the fast onset microencapsulation composition of THC comprises particles having an average size of less than about 70 nm, and the delayed onset microencapsulation composition of THC comprises particles having an average size of more than about 5 μm.

In some embodiments, the fast onset microencapsulation composition of THC comprises particles having an average size of less than about 70 nm, and the delayed onset microencapsulation composition of THC comprises particles having an average size of more than about 6 μm.

In some embodiments, the fast onset microencapsulation composition of THC comprises particles having an average size of less than about 70 nm, and the delayed onset microencapsulation composition of THC comprises particles having an average size of more than about 7 μm.

In some embodiments, the fast onset microencapsulation composition of THC comprises particles having an average size of less than about 70 nm, and the delayed onset microencapsulation composition of THC comprises particles having an average size of more than about 8 μm.

In some embodiments, the fast onset microencapsulation composition of THC comprises particles having an average size of less than about 70 nm, and the delayed onset microencapsulation composition of THC comprises particles having an average size of more than about 9 μm.

In some embodiments, the fast onset microencapsulation composition of THC comprises particles having an average size of less than about 70 nm, and the delayed onset microencapsulation composition of THC comprises particles having an average size of more than about 10 μm.

In some embodiments, the fast onset microencapsulation composition of THC comprises particles having an average size of less than about 60 nm, and the delayed onset microencapsulation composition of TIC comprises particles having an average size of more than about 100 nm.

In some embodiments, the fast onset microencapsulation composition of THC comprises particles having an average size of less than about 60 nm, and the delayed onset microencapsulation composition of THC comprises particles having an average size of more than about 150 nm.

In some embodiments, the fast onset microencapsulation composition of THC comprises particles having an average size of less than about 60 nm, and the delayed onset microencapsulation composition of THC comprises particles having an average size of more than about 200 nm.

In some embodiments, the fast onset microencapsulation composition of THC comprises particles having an average size of less than about 60 nm, and the delayed onset microencapsulation composition of THC comprises particles having an average size of more than about 250 nm.

In some embodiments, the fast onset microencapsulation composition of THC comprises particles having an average size of less than about 60 nm, and the delayed onset microencapsulation composition of THC comprises particles having an average size of more than about 300 nm.

In some embodiments, the fast onset microencapsulation composition of THC comprises particles having an average size of less than about 60 nm, and the delayed onset microencapsulation composition of THC comprises particles having an average size of more than about 350 nm.

In some embodiments, the fast onset microencapsulation composition of THC comprises particles having an average size of less than about 60 nm, and the delayed onset microencapsulation composition of THC comprises particles having an average size of more than about 400 nm.

In some embodiments, the fast onset microencapsulation composition of THC comprises particles having an average size of less than about 60 nm, and the delayed onset microencapsulation composition of TIC comprises particles having an average size of more than about 450 nm.

In some embodiments, the fast onset microencapsulation composition of THC comprises particles having an average size of less than about 60 nm, and the delayed onset microencapsulation composition of THC comprises particles having an average size of more than about 500 nm.

In some embodiments, the fast onset microencapsulation composition of THC comprises particles having an average size of less than about 60 nm, and the delayed onset microencapsulation composition of THC comprises particles having an average size of more than about 600 nm.

In some embodiments, the fast onset microencapsulation composition of THC comprises particles having an average size of less than about 60 nm, and the delayed onset microencapsulation composition of THC comprises particles having an average size of more than about 700 nm.

In some embodiments, the fast onset microencapsulation composition of THC comprises particles having an average size of less than about 60 nm, and the delayed onset microencapsulation composition of THC comprises particles having an average size of more than about 800 nm.

In some embodiments, the fast onset microencapsulation composition of THC comprises particles having an average size of less than about 60 nm, and the delayed onset microencapsulation composition of THC comprises particles having an average size of more than about 900 nm.

In some embodiments, the fast onset microencapsulation composition of THC comprises particles having an average size of less than about 60 nm, and the delayed onset microencapsulation composition of THC comprises particles having an average size of more than about 1 μm.

In some embodiments, the fast onset microencapsulation composition of THC comprises particles having an average size of less than about 60 nm, and the delayed onset microencapsulation composition of THC comprises particles having an average size of more than about 2 μm.

In some embodiments, the fast onset microencapsulation composition of THC comprises particles having an average size of less than about 60 nm, and the delayed onset microencapsulation composition of THC comprises particles having an average size of more than about 3 μm.

In some embodiments, the fast onset microencapsulation composition of THC comprises particles having an average size of less than about 60 nm, and the delayed onset microencapsulation composition of THC comprises particles having an average size of more than about 4 μm.

In some embodiments, the fast onset microencapsulation composition of THC comprises particles having an average size of less than about 60 nm, and the delayed onset microencapsulation composition of THC comprises particles having an average size of more than about 5 μm.

In some embodiments, the fast onset microencapsulation composition of THC comprises particles having an average size of less than about 60 nm, and the delayed onset microencapsulation composition of THC comprises particles having an average size of more than about 6 μm.

In some embodiments, the fast onset microencapsulation composition of THC comprises particles having an average size of less than about 60 nm, and the delayed onset microencapsulation composition of THC comprises particles having an average size of more than about 7 μm.

In some embodiments, the fast onset microencapsulation composition of THC comprises particles having an average size of less than about 60 nm, and the delayed onset microencapsulation composition of THC comprises particles having an average size of more than about 8 μm.

In some embodiments, the fast onset microencapsulation composition of THC comprises particles having an average size of less than about 60 nm, and the delayed onset microencapsulation composition of TIC comprises particles having an average size of more than about 9 μm.

In some embodiments, the fast onset microencapsulation composition of THC comprises particles having an average size of less than about 60 nm, and the delayed onset microencapsulation composition of THC comprises particles having an average size of more than about 10 μm.

In some embodiments, the fast onset microencapsulation composition of THC comprises particles having an average size of less than about 50 nm, and the delayed onset microencapsulation composition of THC comprises particles having an average size of more than about 100 nm.

In some embodiments, the fast onset microencapsulation composition of THC comprises particles having an average size of less than about 50 nm, and the delayed onset microencapsulation composition of THC comprises particles having an average size of more than about 150 nm.

In some embodiments, the fast onset microencapsulation composition of THC comprises particles having an average size of less than about 50 nm, and the delayed onset microencapsulation composition of THC comprises particles having an average size of more than about 200 nm.

In some embodiments, the fast onset microencapsulation composition of THC comprises particles having an average size of less than about 50 nm, and the delayed onset microencapsulation composition of THC comprises particles having an average size of more than about 250 nm.

In some embodiments, the fast onset microencapsulation composition of THC comprises particles having an average size of less than about 50 nm, and the delayed onset microencapsulation composition of THC comprises particles having an average size of more than about 300 nm.

In some embodiments, the fast onset microencapsulation composition of THC comprises particles having an average size of less than about 50 nm, and the delayed onset microencapsulation composition of TIC comprises particles having an average size of more than about 350 nm.

In some embodiments, the fast onset microencapsulation composition of THC comprises particles having an average size of less than about 50 nm, and the delayed onset microencapsulation composition of THC comprises particles having an average size of more than about 400 nm.

In some embodiments, the fast onset microencapsulation composition of THC comprises particles having an average size of less than about 50 nm, and the delayed onset microencapsulation composition of THC comprises particles having an average size of more than about 450 nm.

In some embodiments, the fast onset microencapsulation composition of THC comprises particles having an average size of less than about 50 nm, and the delayed onset microencapsulation composition of THC comprises particles having an average size of more than about 500 nm.

In some embodiments, the fast onset microencapsulation composition of THC comprises particles having an average size of less than about 50 nm, and the delayed onset microencapsulation composition of THC comprises particles having an average size of more than about 600 nm.

In some embodiments, the fast onset microencapsulation composition of THC comprises particles having an average size of less than about 50 nm, and the delayed onset microencapsulation composition of THC comprises particles having an average size of more than about 700 nm.

In some embodiments, the fast onset microencapsulation composition of THC comprises particles having an average size of less than about 50 nm, and the delayed onset microencapsulation composition of THC comprises particles having an average size of more than about 800 nm.

In some embodiments, the fast onset microencapsulation composition of THC comprises particles having an average size of less than about 50 nm, and the delayed onset microencapsulation composition of THC comprises particles having an average size of more than about 900 nm.

In some embodiments, the fast onset microencapsulation composition of THC comprises particles having an average size of less than about 50 nm, and the delayed onset microencapsulation composition of THC comprises particles having an average size of more than about 1 μm.

In some embodiments, the fast onset microencapsulation composition of THC comprises particles having an average size of less than about 50 nm, and the delayed onset microencapsulation composition of THC comprises particles having an average size of more than about 2 μm.

In some embodiments, the fast onset microencapsulation composition of THC comprises particles having an average size of less than about 50 nm, and the delayed onset microencapsulation composition of THC comprises particles having an average size of more than about 3 μm.

In some embodiments, the fast onset microencapsulation composition of THC comprises particles having an average size of less than about 50 nm, and the delayed onset microencapsulation composition of THC comprises particles having an average size of more than about 4 μm.

In some embodiments, the fast onset microencapsulation composition of THC comprises particles having an average size of less than about 50 nm, and the delayed onset microencapsulation composition of THC comprises particles having an average size of more than about 5 μm.

In some embodiments, the fast onset microencapsulation composition of THC comprises particles having an average size of less than about 50 nm, and the delayed onset microencapsulation composition of THC comprises particles having an average size of more than about 6 μm.

In some embodiments, the fast onset microencapsulation composition of THC comprises particles having an average size of less than about 50 nm, and the delayed onset microencapsulation composition of TIC comprises particles having an average size of more than about 7 μm.

In some embodiments, the fast onset microencapsulation composition of THC comprises particles having an average size of less than about 50 nm, and the delayed onset microencapsulation composition of THC comprises particles having an average size of more than about 8 μm.

In some embodiments, the fast onset microencapsulation composition of THC comprises particles having an average size of less than about 50 nm, and the delayed onset microencapsulation composition of THC comprises particles having an average size of more than about 9 μm.

In some embodiments, the fast onset microencapsulation composition of THC comprises particles having an average size of less than about 50 nm, and the delayed onset microencapsulation composition of THC comprises particles having an average size of more than about 10 μm.

In some embodiments, the fast onset microencapsulation composition of THC comprises particles having an average size of less than about 40 nm, and the delayed onset microencapsulation composition of THC comprises particles having an average size of more than about 100 nm.

In some embodiments, the fast onset microencapsulation composition of THC comprises particles having an average size of less than about 40 nm, and the delayed onset microencapsulation composition of THC comprises particles having an average size of more than about 150 nm.

In some embodiments, the fast onset microencapsulation composition of THC comprises particles having an average size of less than about 40 nm, and the delayed onset microencapsulation composition of THC comprises particles having an average size of more than about 200 nm.

In some embodiments, the fast onset microencapsulation composition of THC comprises particles having an average size of less than about 40 nm, and the delayed onset microencapsulation composition of THC comprises particles having an average size of more than about 250 nm.

In some embodiments, the fast onset microencapsulation composition of THC comprises particles having an average size of less than about 40 nm, and the delayed onset microencapsulation composition of THC comprises particles having an average size of more than about 300 nm.

In some embodiments, the fast onset microencapsulation composition of THC comprises particles having an average size of less than about 40 nm, and the delayed onset microencapsulation composition of THC comprises particles having an average size of more than about 350 nm.

In some embodiments, the fast onset microencapsulation composition of THC comprises particles having an average size of less than about 40 nm, and the delayed onset microencapsulation composition of THC comprises particles having an average size of more than about 400 nm.

In some embodiments, the fast onset microencapsulation composition of THC comprises particles having an average size of less than about 40 nm, and the delayed onset microencapsulation composition of THC comprises particles having an average size of more than about 450 nm.

In some embodiments, the fast onset microencapsulation composition of THC comprises particles having an average size of less than about 40 nm, and the delayed onset microencapsulation composition of THC comprises particles having an average size of more than about 500 nm.

In some embodiments, the fast onset microencapsulation composition of THC comprises particles having an average size of less than about 40 nm, and the delayed onset microencapsulation composition of THC comprises particles having an average size of more than about 600 nm.

In some embodiments, the fast onset microencapsulation composition of THC comprises particles having an average size of less than about 40 nm, and the delayed onset microencapsulation composition of TIC comprises particles having an average size of more than about 700 nm.

In some embodiments, the fast onset microencapsulation composition of THC comprises particles having an average size of less than about 40 nm, and the delayed onset microencapsulation composition of THC comprises particles having an average size of more than about 800 nm.

In some embodiments, the fast onset microencapsulation composition of THC comprises particles having an average size of less than about 40 nm, and the delayed onset microencapsulation composition of THC comprises particles having an average size of more than about 900 nm.

In some embodiments, the fast onset microencapsulation composition of THC comprises particles having an average size of less than about 40 nm, and the delayed onset microencapsulation composition of THC comprises particles having an average size of more than about 1 μm.

In some embodiments, the fast onset microencapsulation composition of THC comprises particles having an average size of less than about 40 nm, and the delayed onset microencapsulation composition of THC comprises particles having an average size of more than about 2 μm.

In some embodiments, the fast onset microencapsulation composition of THC comprises particles having an average size of less than about 40 nm, and the delayed onset microencapsulation composition of THC comprises particles having an average size of more than about 3 μm.

In some embodiments, the fast onset microencapsulation composition of THC comprises particles having an average size of less than about 40 nm, and the delayed onset microencapsulation composition of THC comprises particles having an average size of more than about 4 μm.

In some embodiments, the fast onset microencapsulation composition of THC comprises particles having an average size of less than about 40 nm, and the delayed onset microencapsulation composition of THC comprises particles having an average size of more than about 5 μm.

In some embodiments, the fast onset microencapsulation composition of THC comprises particles having an average size of less than about 40 nm, and the delayed onset microencapsulation composition of THC comprises particles having an average size of more than about 6 μm.

In some embodiments, the fast onset microencapsulation composition of THC comprises particles having an average size of less than about 40 nm, and the delayed onset microencapsulation composition of THC comprises particles having an average size of more than about 7 μm.

In some embodiments, the fast onset microencapsulation composition of THC comprises particles having an average size of less than about 40 nm, and the delayed onset microencapsulation composition of THC comprises particles having an average size of more than about 8 μm.

In some embodiments, the fast onset microencapsulation composition of THC comprises particles having an average size of less than about 40 nm, and the delayed onset microencapsulation composition of THC comprises particles having an average size of more than about 9 μm.

In some embodiments, the fast onset microencapsulation composition of THC comprises particles having an average size of less than about 40 nm, and the delayed onset microencapsulation composition of THC comprises particles having an average size of more than about 10 μm.

In some embodiments, the fast onset microencapsulation composition of THC comprises particles having an average size of less than about 30 nm, and the delayed onset microencapsulation composition of THC comprises particles having an average size of more than about 100 nm.

In some embodiments, the fast onset microencapsulation composition of THC comprises particles having an average size of less than about 30 nm, and the delayed onset microencapsulation composition of THC comprises particles having an average size of more than about 150 nm.

In some embodiments, the fast onset microencapsulation composition of THC comprises particles having an average size of less than about 30 nm, and the delayed onset microencapsulation composition of THC comprises particles having an average size of more than about 200 nm.

In some embodiments, the fast onset microencapsulation composition of THC comprises particles having an average size of less than about 30 nm, and the delayed onset microencapsulation composition of THC comprises particles having an average size of more than about 250 nm.

In some embodiments, the fast onset microencapsulation composition of THC comprises particles having an average size of less than about 30 nm, and the delayed onset microencapsulation composition of THC comprises particles having an average size of more than about 300 nm.

In some embodiments, the fast onset microencapsulation composition of THC comprises particles having an average size of less than about 30 nm, and the delayed onset microencapsulation composition of THC comprises particles having an average size of more than about 350 nm.

In some embodiments, the fast onset microencapsulation composition of THC comprises particles having an average size of less than about 30 nm, and the delayed onset microencapsulation composition of THC comprises particles having an average size of more than about 400 nm.

In some embodiments, the fast onset microencapsulation composition of THC comprises particles having an average size of less than about 30 nm, and the delayed onset microencapsulation composition of THC comprises particles having an average size of more than about 450 nm.

In some embodiments, the fast onset microencapsulation composition of THC comprises particles having an average size of less than about 30 nm, and the delayed onset microencapsulation composition of TIC comprises particles having an average size of more than about 500 nm.

In some embodiments, the fast onset microencapsulation composition of THC comprises particles having an average size of less than about 30 nm, and the delayed onset microencapsulation composition of THC comprises particles having an average size of more than about 600 nm.

In some embodiments, the fast onset microencapsulation composition of THC comprises particles having an average size of less than about 30 nm, and the delayed onset microencapsulation composition of THC comprises particles having an average size of more than about 700 nm.

In some embodiments, the fast onset microencapsulation composition of THC comprises particles having an average size of less than about 30 nm, and the delayed onset microencapsulation composition of THC comprises particles having an average size of more than about 800 nm.

In some embodiments, the fast onset microencapsulation composition of THC comprises particles having an average size of less than about 30 nm, and the delayed onset microencapsulation composition of THC comprises particles having an average size of more than about 900 nm.

In some embodiments, the fast onset microencapsulation composition of THC comprises particles having an average size of less than about 30 nm, and the delayed onset microencapsulation composition of THC comprises particles having an average size of more than about 1 μm.

In some embodiments, the fast onset microencapsulation composition of THC comprises particles having an average size of less than about 30 nm, and the delayed onset microencapsulation composition of THC comprises particles having an average size of more than about 2 μm.

In some embodiments, the fast onset microencapsulation composition of THC comprises particles having an average size of less than about 30 nm, and the delayed onset microencapsulation composition of TIC comprises particles having an average size of more than about 3 μm.

In some embodiments, the fast onset microencapsulation composition of THC comprises particles having an average size of less than about 30 nm, and the delayed onset microencapsulation composition of THC comprises particles having an average size of more than about 4 μm.

In some embodiments, the fast onset microencapsulation composition of THC comprises particles having an average size of less than about 30 nm, and the delayed onset microencapsulation composition of THC comprises particles having an average size of more than about 5 μm.

In some embodiments, the fast onset microencapsulation composition of THC comprises particles having an average size of less than about 30 nm, and the delayed onset microencapsulation composition of THC comprises particles having an average size of more than about 6 μm.

In some embodiments, the fast onset microencapsulation composition of THC comprises particles having an average size of less than about 30 nm, and the delayed onset microencapsulation composition of THC comprises particles having an average size of more than about 7 μm.

In some embodiments, the fast onset microencapsulation composition of THC comprises particles having an average size of less than about 30 nm, and the delayed onset microencapsulation composition of THC comprises particles having an average size of more than about 8 μm.

In some embodiments, the fast onset microencapsulation composition of THC comprises particles having an average size of less than about 30 nm, and the delayed onset microencapsulation composition of THC comprises particles having an average size of more than about 9 μm.

In some embodiments, the fast onset microencapsulation composition of THC comprises particles having an average size of less than about 30 nm, and the delayed onset microencapsulation composition of TIC comprises particles having an average size of more than about 10 μm.

In some embodiments, the fast onset microencapsulation composition of THC comprises particles having an average size of less than about 20 nm, and the delayed onset microencapsulation composition of THC comprises particles having an average size of more than about 100 nm.

In some embodiments, the fast onset microencapsulation composition of THC comprises particles having an average size of less than about 20 nm, and the delayed onset microencapsulation composition of THC comprises particles having an average size of more than about 150 nm.

In some embodiments, the fast onset microencapsulation composition of THC comprises particles having an average size of less than about 20 nm, and the delayed onset microencapsulation composition of THC comprises particles having an average size of more than about 200 nm.

In some embodiments, the fast onset microencapsulation composition of THC comprises particles having an average size of less than about 20 nm, and the delayed onset microencapsulation composition of THC comprises particles having an average size of more than about 250 nm.

In some embodiments, the fast onset microencapsulation composition of THC comprises particles having an average size of less than about 20 nm, and the delayed onset microencapsulation composition of THC comprises particles having an average size of more than about 300 nm.

In some embodiments, the fast onset microencapsulation composition of THC comprises particles having an average size of less than about 20 nm, and the delayed onset microencapsulation composition of THC comprises particles having an average size of more than about 350 nm.

In some embodiments, the fast onset microencapsulation composition of THC comprises particles having an average size of less than about 20 nm, and the delayed onset microencapsulation composition of TIC comprises particles having an average size of more than about 400 nm.

In some embodiments, the fast onset microencapsulation composition of THC comprises particles having an average size of less than about 20 nm, and the delayed onset microencapsulation composition of THC comprises particles having an average size of more than about 450 nm.

In some embodiments, the fast onset microencapsulation composition of THC comprises particles having an average size of less than about 20 nm, and the delayed onset microencapsulation composition of THC comprises particles having an average size of more than about 500 nm.

In some embodiments, the fast onset microencapsulation composition of THC comprises particles having an average size of less than about 20 nm, and the delayed onset microencapsulation composition of THC comprises particles having an average size of more than about 600 nm.

In some embodiments, the fast onset microencapsulation composition of THC comprises particles having an average size of less than about 20 nm, and the delayed onset microencapsulation composition of THC comprises particles having an average size of more than about 700 nm.

In some embodiments, the fast onset microencapsulation composition of THC comprises particles having an average size of less than about 20 nm, and the delayed onset microencapsulation composition of THC comprises particles having an average size of more than about 800 nm.

In some embodiments, the fast onset microencapsulation composition of THC comprises particles having an average size of less than about 20 nm, and the delayed onset microencapsulation composition of THC comprises particles having an average size of more than about 900 nm.

In some embodiments, the fast onset microencapsulation composition of THC comprises particles having an average size of less than about 20 nm, and the delayed onset microencapsulation composition of THC comprises particles having an average size of more than about 1 μm.

In some embodiments, the fast onset microencapsulation composition of THC comprises particles having an average size of less than about 20 nm, and the delayed onset microencapsulation composition of THC comprises particles having an average size of more than about 2 μm.

In some embodiments, the fast onset microencapsulation composition of THC comprises particles having an average size of less than about 20 nm, and the delayed onset microencapsulation composition of THC comprises particles having an average size of more than about 3 μm.

In some embodiments, the fast onset microencapsulation composition of THC comprises particles having an average size of less than about 20 nm, and the delayed onset microencapsulation composition of THC comprises particles having an average size of more than about 4 μm.

In some embodiments, the fast onset microencapsulation composition of THC comprises particles having an average size of less than about 20 nm, and the delayed onset microencapsulation composition of THC comprises particles having an average size of more than about 5 μm.

In some embodiments, the fast onset microencapsulation composition of THC comprises particles having an average size of less than about 20 nm, and the delayed onset microencapsulation composition of THC comprises particles having an average size of more than about 6 μm.

In some embodiments, the fast onset microencapsulation composition of THC comprises particles having an average size of less than about 20 nm, and the delayed onset microencapsulation composition of THC comprises particles having an average size of more than about 7 μm.

In some embodiments, the fast onset microencapsulation composition of THC comprises particles having an average size of less than about 20 nm, and the delayed onset microencapsulation composition of THC comprises particles having an average size of more than about 8 μm.

In some embodiments, the fast onset microencapsulation composition of THC comprises particles having an average size of less than about 20 nm, and the delayed onset microencapsulation composition of THC comprises particles having an average size of more than about 9 μm.

In some embodiments, the fast onset microencapsulation composition of THC comprises particles having an average size of less than about 20 nm, and the delayed onset microencapsulation composition of THC comprises particles having an average size of more than about 10 μm.

In some embodiments, the fast onset microencapsulation composition of THC comprises particles having an average size of less than about 10 nm, and the delayed onset microencapsulation composition of THC comprises particles having an average size of more than about 100 nm.

In some embodiments, the fast onset microencapsulation composition of THC comprises particles having an average size of less than about 10 nm, and the delayed onset microencapsulation composition of THC comprises particles having an average size of more than about 150 nm.

In some embodiments, the fast onset microencapsulation composition of THC comprises particles having an average size of less than about 10 nm, and the delayed onset microencapsulation composition of THC comprises particles having an average size of more than about 200 nm.

In some embodiments, the fast onset microencapsulation composition of THC comprises particles having an average size of less than about 10 nm, and the delayed onset microencapsulation composition of THC comprises particles having an average size of more than about 250 nm.

In some embodiments, the fast onset microencapsulation composition of THC comprises particles having an average size of less than about 10 nm, and the delayed onset microencapsulation composition of TIC comprises particles having an average size of more than about 300 nm.

In some embodiments, the fast onset microencapsulation composition of THC comprises particles having an average size of less than about 10 nm, and the delayed onset microencapsulation composition of THC comprises particles having an average size of more than about 350 nm.

In some embodiments, the fast onset microencapsulation composition of THC comprises particles having an average size of less than about 10 nm, and the delayed onset microencapsulation composition of THC comprises particles having an average size of more than about 400 nm.

In some embodiments, the fast onset microencapsulation composition of THC comprises particles having an average size of less than about 10 nm, and the delayed onset microencapsulation composition of THC comprises particles having an average size of more than about 450 nm.

In some embodiments, the fast onset microencapsulation composition of THC comprises particles having an average size of less than about 10 nm, and the delayed onset microencapsulation composition of THC comprises particles having an average size of more than about 500 nm.

In some embodiments, the fast onset microencapsulation composition of THC comprises particles having an average size of less than about 10 nm, and the delayed onset microencapsulation composition of THC comprises particles having an average size of more than about 600 nm.

In some embodiments, the fast onset microencapsulation composition of THC comprises particles having an average size of less than about 10 nm, and the delayed onset microencapsulation composition of THC comprises particles having an average size of more than about 700 nm.

In some embodiments, the fast onset microencapsulation composition of THC comprises particles having an average size of less than about 10 nm, and the delayed onset microencapsulation composition of THC comprises particles having an average size of more than about 800 nm.

In some embodiments, the fast onset microencapsulation composition of THC comprises particles having an average size of less than about 10 nm, and the delayed onset microencapsulation composition of THC comprises particles having an average size of more than about 900 nm.

In some embodiments, the fast onset microencapsulation composition of THC comprises particles having an average size of less than about 10 nm, and the delayed onset microencapsulation composition of THC comprises particles having an average size of more than about 1 μm.

In some embodiments, the fast onset microencapsulation composition of THC comprises particles having an average size of less than about 10 nm, and the delayed onset microencapsulation composition of THC comprises particles having an average size of more than about 2 μm.

In some embodiments, the fast onset microencapsulation composition of THC comprises particles having an average size of less than about 10 nm, and the delayed onset microencapsulation composition of THC comprises particles having an average size of more than about 3 μm.

In some embodiments, the fast onset microencapsulation composition of THC comprises particles having an average size of less than about 10 nm, and the delayed onset microencapsulation composition of THC comprises particles having an average size of more than about 4 μm.

In some embodiments, the fast onset microencapsulation composition of THC comprises particles having an average size of less than about 10 nm, and the delayed onset microencapsulation composition of THC comprises particles having an average size of more than about 5 μm.

In some embodiments, the fast onset microencapsulation composition of THC comprises particles having an average size of less than about 10 nm, and the delayed onset microencapsulation composition of THC comprises particles having an average size of more than about 6 μm.

In some embodiments, the fast onset microencapsulation composition of THC comprises particles having an average size of less than about 10 nm, and the delayed onset microencapsulation composition of THC comprises particles having an average size of more than about 7 μm.

In some embodiments, the fast onset microencapsulation composition of THC comprises particles having an average size of less than about 10 nm, and the delayed onset microencapsulation composition of THC comprises particles having an average size of more than about 8 μm.

In some embodiments, the fast onset microencapsulation composition of THC comprises particles having an average size of less than about 10 nm, and the delayed onset microencapsulation composition of THC comprises particles having an average size of more than about 9 μm.

In some embodiments, the fast onset microencapsulation composition of THC comprises particles having an average size of less than about 10 nm, and the delayed onset microencapsulation composition of THC comprises particles having an average size of more than about 10 μm.

### 10. Cannabis-Infused Edibles

Cannabis-infused edibles are food products infused with cannabis extracts (such as oil), which contain a cannabinoid profile and which are made of a non-liquid edible matrix.

Edibles come in many forms and can be any product that is suitable, e.g., non-toxic, for placing into the mouth of a human, whether ingested, absorbed, or only chewed or sucked on and at least a portion discarded, etc. Illustrative examples of human edible products include chewing or bubble gums, mints, suckers, jawbreakers, lozenges, hard candies, gummy candies, taffies, chocolates, baked goods such as muffins, brownies, cookies, crackers, granola or meal replacement bars, smokeless inhalation powders, honey, syrup, spreads, and dissolving strips. For example, a chewing-gum may have a hard shell (akin an Excel™ chewing-gum) or not (akin Juicy Fruit™).

The person of skill will readily understand how to infuse a product base to obtain the herein described cannabis-infused product.

For example, in order to infuse a chewing gum base, the person of skill may proceed to contact and mix the chewing gum ingredients (such as gum base [e.g., elastomers, waxes, and resin], sweeteners, glycerin, plasticizer and colors) with an embodiment of the herein described precursor composition and process the mixture to obtain the cannabis-infused chewing gum.

For example, the person of skill may mix an embodiment of the herein described microencapsulating composition including 0.69 wt. % THC with a chewing gum base (product base) including: 75.5 wt. % gum base, xylitol 14 wt. %, glycerin 4.5 wt. %, saccharine 0.38 wt. %, peppermint aroma oil 1.5 wt. %, peppermint powder 1.5 wt. %, and water 2.3 wt. % in order to obtain a 10 mg THC gum.

### 11. Cannabis-Infused Liquid Compositions

Cannabis-infused liquid compositions are liquid compositions products which can be used in many liquid applications. For example, such cannabis-infused liquid composition can be used for ingestion or application to a user's skin or mucous membrane.

The liquid compositions may come in many forms—including but without being limited to beverages, gels, creams, custard, pudding, honey, syrup, broth, soup, gelatin, yogurt, puree, jelly, sauce, liquid eggs, or salad dressing.

The liquid compositions may be adapted for topical administration to the skin or mucous membrane. For topical administration, the liquid composition may be prepared as an ointment, tincture, cream, gel, solution including a mouth wash, lotion, spray, aerosol, dry powder for inhalation, suspension, and the like. A preparation for topical administration to the skin can be prepared by mixing the liquid composition with non-toxic, therapeutically inert, solid or liquid carriers customarily used in such preparations.

The liquid composition may be a beverage which includes, but without being limited to, drinking water, milk (both diary and non-diary), juice, a smoothie, coffee or a caffeinated beverage, tea, herbal tea, a cocoa beverage, a carbonated drink, a nitrogenated drink, an energy drink, a drinkable yogurt, a fermented beverage, or an alcoholic or non-alcoholic drink. An alcoholic or non-alcoholic drink includes but is not limited to, beer, lager, cider, spirits, wine/fortified wine, and cocktails.

The person of skill will readily understand how to infuse a product base to obtain the herein described cannabis-infused product.

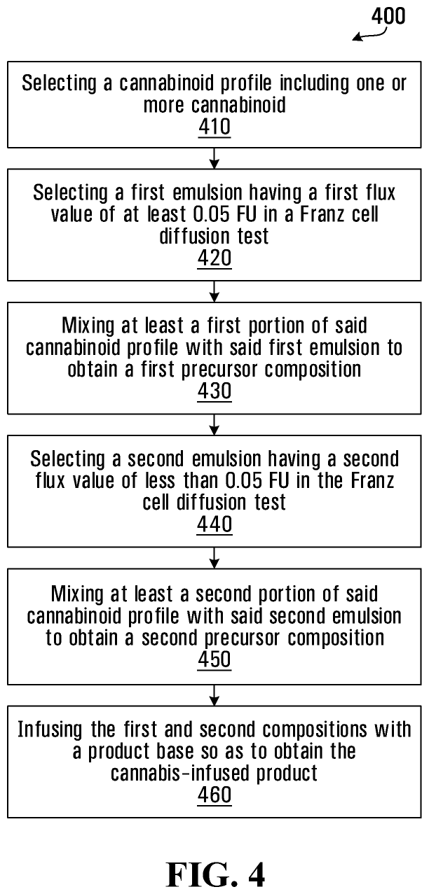

With reference to FIG. 4, there is shown a non-limiting flowchart that sets out a process 400 for manufacturing a cannabis-infused product as per an embodiment of the present disclosure. For example, the process 400 includes a step 410 of selecting a cannabinoid profile including one or more cannabinoid. As discussed elsewhere, this cannabinoid profile may also further include other cannabis-derived products such as terpenes, flavonoids, and the like. The process 400 further includes a step 420 of selecting a first emulsion having specific characteristics which are desired for at least a portion of the cannabinoid profile. For example, the first emulsion may have a flux value of at least 0.05 FU in a Franz cell diffusion test, as will be further discussed later in this text. The at least portion of the cannabinoid profile and the first emulsion are then mixed in a step 430 to obtain a first precursor composition. The process 400 further includes a step 440 of selecting a second emulsion having specific characteristics which are desired for at least a second portion of the cannabinoid profile. For example, the second emulsion may have a flux value of less than 0.05 FU in the Franz cell diffusion test, as will be further discussed later in this text. The at least second portion of the cannabinoid profile and the second emulsion are then mixed in a step 450 to obtain a second precursor composition. The first and second precursor compositions are then mixed sequentially or concomitantly with a product base in a step 460 to obtain the cannabis-infused product.

It will be apparent to the person of skill that while processes 400 has been described with a number of sequential steps, other variations may be possible where, e.g., one or more of the above discussed steps can be performed concomitantly with one or more other steps rather than sequentially, and such variations are within the scope of the present disclosure.

For example, in some embodiments, dilution or infusion of the herein described precursor in a cannabinoid-less beverage or blending with a beverage base can result in a beverage product comprising at least 0.002 mg/ml of cannabinoid in total volume of the beverage product. For example, the beverage product may include from 0.002 mg/ml to about 1 mg/ml of cannabinoid in volume of the beverage product.

For example, the person of skill may mix an embodiment of the herein described precursor composition including 20 mg/ml THC with a brewed beer (product base) in order to obtain a cannabis-infused beer, which can be canned in a container in an amount sufficient to have, for example but without being limited to, 10 mg of THC per container (e.g., a container of 355 ml).

In some embodiments, the cannabis-infused liquid composition provided herein can be a beverage contained in a packaging unit, the unit comprising less than 1000 mg, or less than 900 mg, or less than 800 mg, or less than 700 mg, or less than 600 mg, or less than 500 mg, or less than 400 mg, or less than 300 mg, or less than 200 mg, or less than 100 mg, or less than 50 mg, or less than 40 mg, or less than 30 mg, or less than 20 mg, or less than 10 mg, or less than 5 mg, or less than 2.5 mg of a specific cannabis extract such as THC, CBD, terpene (e.g., D-limonene) or any mixtures thereof. In some embodiments, the beverage may include, for example, per packaging unit up to 1 g, up to 750 mg, up to 700 mg, up to 650 mg, up to 600 mg, up to 550 mg, up to 500 mg, up to 450 mg, up to 400 mg, up to 350 mg, up to 300 mg, up to 250 mg, up to 200 mg, up to 150 mg, up to 100 mg, up to 50 mg, up to 40 mg, up to 35 mg, up to 30 mg, up to 25 mg, up to 20 mg, up to 15 mg, up to 10 mg, up to 9 mg, up to 8 mg, up to 7 mg, up to 6 mg, up to 5 mg, up to 4 mg, up to 3 mg, up to 2 mg, or up to 1 mg of a specific cannabis extract such as THC, CBD, terpene (e.g., D-limonene) or any mixtures thereof, and the like.

In some embodiments, dilution or infusion of the herein described precursor compositions in a product base (e.g., cannabinoid-less beverage or blending with a beverage base) results in a cannabis-infused liquid composition comprising at least 0.002 mg/ml of cannabinoid in volume of the cannabis-infused liquid composition, the cannabis-infused liquid composition having a viscosity of at least 50 mPas, or up to 1500 mPas, e.g., selected in the range of from 50 mPas (for juice-like beverages) to 1500 mPas (for more honey-like beverages, such as fruit juice concentrates) measured at room temperature (e.g., 25° C.). In some embodiments, the cannabis-infused liquid composition may have a viscosity which is substantially the same as that one of the product base. The person of skill will readily understand how to assess viscosity of a cannabis-infused liquid composition, for example with the use of a rheometer, such as the RheolabQC (Anton Parr, Canada).

### 11. Methods of Clarifying a Cannabis-Infused Liquid Composition

In some embodiments, it may be desirable for a cannabis-infused liquid composition to have a certain degree of clarity, for example in cases where the cannabis-infused product may be more appealing to the consumer when the product retains its initial clarity as this may convey some sort of freshness or palatability perception to the consumer. There are many options for ensuring that the cannabis-infused liquid composition retains the desired clarity characteristic.

In some embodiments, the herein described cannabis-infused liquid composition may thus be clear, translucent or transparent.

The appearance of a liquid containing an emulsion usually depends on the scattering of light by the emulsion droplets and the absorption of light by any chromophores present. In some embodiments, for clear liquids, the majority of droplets should be less than approximately 50 nm in diameter so that light scattering is very weak.

In some embodiments, a turbidity (or “cloudiness”) of less than 0.05 cm⁻¹ (at 600 nm) as measured with a spectrophotometer is generally considered to be an approximate cut-off point between transparent and cloudy beverage.

In some embodiments, a turbidity (or “cloudiness”) of less than 30 Nephelometric Turbidity Units (NTU) as measured with a nephelometer is, additionally or alternatively, generally considered to be an approximate cut-off point between transparent and cloudy beverage.

In some embodiments, the cannabis-infused liquid composition provided herein has a turbidity of less than about 0.05 cm⁻¹ measured at a wavelength of 600 nm. In some embodiments, the cannabis-infused liquid composition provided herein may have a turbidity of less than about 0.04 cm⁻¹ measured at a wavelength of 600 nm. In some embodiments, the cannabis-infused liquid composition provided herein may have a turbidity of less than about 0.03 cm⁻¹ measured at a wavelength of 600 nm. In some embodiments, the cannabis-infused liquid composition provided herein may have a turbidity of less than about 0.02 cm⁻¹ measured at a wavelength of 600 nm. In some embodiments, the cannabis-infused liquid composition provided herein may have a turbidity of less than about 0.01 cm⁻¹ measured at a wavelength of 600 nm.

In some embodiments, the cannabis-infused liquid composition provided herein may be processed to improve the appearance thereof. For example, the cannabis-infused liquid composition comprising a cannabinoid profile may be blended with a fining agent under fining conditions so as to have a turbidity of less than about 0.05 cm⁻¹ measured at a wavelength of 600 nm and/or less than 30 NTU. Fining agents are known in the art, and may include for example an agent selected from bentonite, gelatin, casein, carrageenan, alginate, diatomaceous earth, pectinase, pectolyase, PVPP, kieselsol (colloidal silica), copper sulfate, dried albumen, hydrated yeast, and activated carbon. In some embodiments, the fining agent includes gelatin.

In some embodiments, dilution or infusion of the herein described precursor compositions in a cannabinoid-less beverage or blending with a beverage base results in a beverage comprising at least 0.002 mg/ml of cannabinoid in volume of the beverage, the beverage having a turbidity of less than 0.05 cm−1 at 600 nm and/or less than 30 NTU.

The cannabis-infused liquid composition can then optionally be further processed, for example, by storing under a suitable temperature such as a temperature S 4° C., for example −20° C., for a suitable period of time. For example, a suitable period of time may include at least 30 minutes, at least 1 h, at least 2 h, at least 3 h, at least 4 h, at least 5 h, at least 12 h, at least 24 h, at least 48 h, at least 72 h.

The cannabis-infused liquid composition obtained thereafter can then be recovered under suitable conditions. For example, one of skill may implement filtering techniques or any other means known in the art to discard the sedimentation.

In some embodiments, the fining agent includes gelatin which can be used at a concentration of ≤2% (wt./wt.), or at a concentration of ≤1% (wt./wt.), or at a concentration of ≤0.8% (wt./wt.). For example, the gelatin can be used at a concentration of ≥0.05% (wt./wt.), or ≥0.1% (wt./wt.), or ≥0.2% (wt./wt.), or ≥0.3% (wt./wt.), or ≥0.4% (wt./wt.), or≥0.5% (wt./wt.), or ≥0.6% (wt./wt.), or ≥0.7% (wt./wt.).

In some embodiments, the fining agent includes gelatin which can be used at a concentration included in the range of 0.8% to 1% (wt./wt.).

### 12. Test Procedures

### 12.1 Franz Cell Test

The onset characteristic of a cannabinoid profile or of the corresponding attenuator, modulator or antidote can be assessed in the context of a liquid composition using its permeation across an ex vivo biological membrane as an indicator of the time required to reach the user's bloodstream after contacting the user's skin or mucous membrane (e.g., after ingestion).

The onset characteristic can be measured using a Franz cell diffusion test which is designed to measure the permeation of a cannabinoid profile or of the corresponding attenuator, modulator or antidote across ex vivo biological membranes. In this test, the biological membranes used are harvested membranes which are essentially metabolically deactivated; there are no active transporter enzymes and permeation across the membrane, thus, relies on passive diffusion mechanisms. The biological membrane used in this specification is porcine oral mucosa due to its similarity to human lipid and protein membrane composition and one can thus reasonably infer from data obtained with this test how a liquid composition (such as a beverage) containing the cannabinoid profile will behave in terms of delivering the cannabinoid profile to a user having ingested the liquid composition.

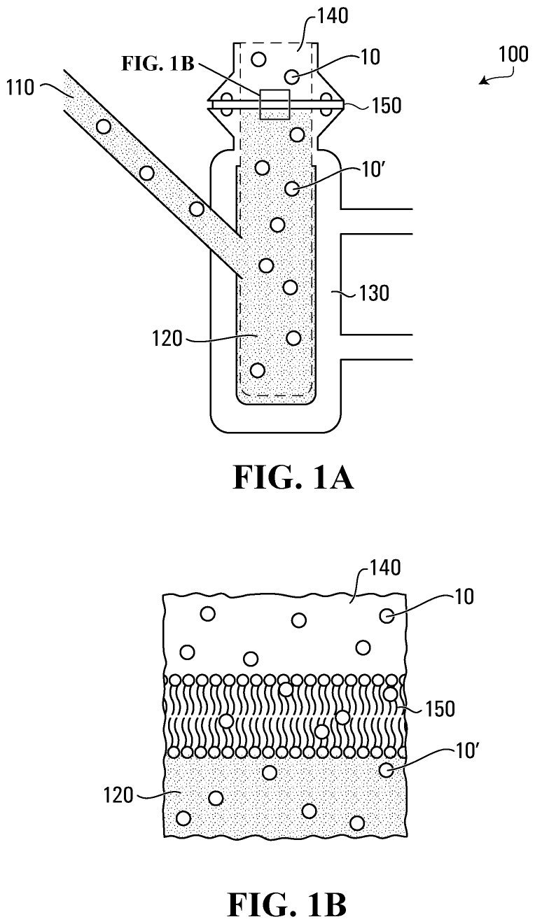

FIG. 1 illustrates a practical non-limiting embodiment of a Franz cell diffusion device 100 used in this example. The Franz cell diffusion device 100 includes a donor cell 140 and a receptor cell 120 separated by a biological membrane 150, which can be for example porcine oral mucosa freshly harvested and stored in buffer. The receptor cell 120 includes a sampling outlet 110 in fluid communication with the receptor cell 120 to allow taking samples from the receptor cell 120. The Franz cell diffusion device 100 may further include a thermal jacket 130 to maintain a pre-determined temperature for the test, which can be for example about 37° C.

The test procedure is as follows:
a. A volume of 1 ml of a first liquid composition containing the first portion of the cannabinoid profile 10 and having a starting cannabinoid concentration [C]ₛ is provided (measured using suitable instrumentation, e.g. HPLC).
b. The test Franz cell diffusion device 100 is provided. The receptor cell 120 is initially loaded with saliva phosphate buffer pH 6.2 and the Franz cell diffusion device 100 is maintained at 37° C.
c. The 1 ml of the first liquid composition is applied to the donor cell 140, and allowed to diffuse across the membrane 150 during a period of time of 2.5 h.
d. A sample containing the cannabinoid profile 10′ having diffused across the membrane 150 is then taken over from the receptor cell 120 through sampling outlet 110. The end cannabinoid concentration [C]_{E} in the sample is measured (using suitable instrumentation, e.g. HPLC).
   (1) The results can be reported in either [C]_{E} or as “flux”. Flux consists of calculating the cannabinoid as flux units (FU) where 1 FU means a calculated value of 1 μg/cm²/h.
e. The same procedure can be applied to a second liquid composition containing the second portion of the cannabinoid profile.

Note that for the purpose of the present description, the above defined test procedure will be referred to as “Franz cell test”.

According to the present disclosure, the cannabis-infused liquid composition includes a composition that has a flux value in the Franz cell test of at least 0.05 FU, preferably of at least 0.08 FU), more preferably of at least 0.10 FU, more preferably of at least 0.20 FU, more preferably of at least 0.28 FU, even more preferably of at least 0.30 FU. Without being bound by any theory, the present inventors predict that based on the results generated and the teachings of the present specification, an emulsion (or a cannabis-infused liquid containing an emulsion) having such flux value in the Franz cell test should afford a number of advantages such as a faster onset of the cannabis-associated effect compared to an identical emulsion (or cannabis-infused liquid containing an emulsion) but having a different flux value.

According to the present disclosure, the cannabis-infused liquid composition includes a composition that has a flux value in the Franz cell test of less than 0.05 FU, preferably of less than 0.025 FU, more preferably of less than 0.010 FU. Without being bound by any theory, the present inventors predict that based on the results generated and the teachings of the present specification, an emulsion (or a cannabis-infused liquid containing an emulsion) having such flux value in the Franz cell test should afford a number of advantages such as a delayed onset of the cannabis-associated effect compared to an identical emulsion (or cannabis-infused liquid containing an emulsion) but having a different flux value.

### 12.2 Tissue Cell Permeation Test

The onset characteristic of a cannabinoid profile or of the corresponding attenuator, modulator or antidote can be assessed in the context of a liquid composition using its permeation across a biologically active tissue cells as an indicator of the time required to reach the user's bloodstream after contacting the user's skin or mucous membrane (e.g., after ingestion).

The onset characteristic can be measured using a tissue cell permeation test, which is designed to evaluate cannabinoid profile or of the corresponding attenuator, modulator or antidote absorption through tissue cells. In this test, the tissue cells are essentially metabolically active; there are active transporter enzymes and permeation across the membrane, thus, relies mainly on active diffusion mechanisms. The tissue cells are oral or intestinal membrane cells grown in culture and one can thus, similarly to the Franz cell data, reasonably infer from data obtained with this test how a liquid composition (such as a beverage) containing the cannabinoid profile and corresponding antidote, attenuator or modulator will behave in terms of the cannabinoid profile onset/offset in a user having been administered the liquid composition.

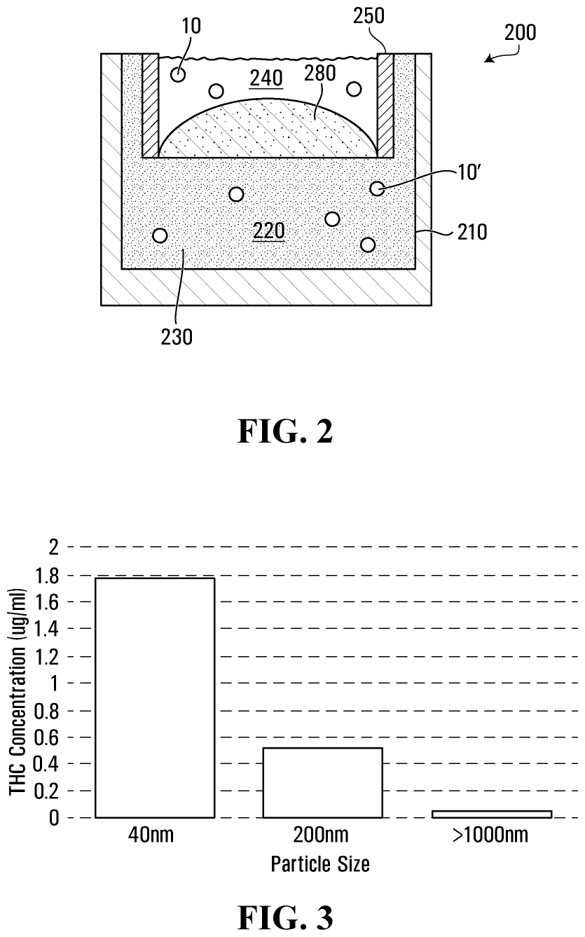

FIG. 2 illustrates a practical non-limiting embodiment of a tissue cell permeation test device 200 used in this example. The tissue cell permeation test device 200 includes oral or intestinal membrane cells 280 cells grown on the bottom of a well insert 250 which defines a donor chamber 240. The well insert 250 is contained in a larger well 210 and floats over cell culture media serum 230 contained in the larger well 210. The larger well 210 defines a receptor chamber 220. Viable cells 280 are grown within the well insert 250 effectively creating a living membrane containing active transport mechanisms.

The test procedure is as follows:
a. A first liquid composition containing the first portion of the cannabinoid profile 10 having a starting cannabinoid concentration [C]ₛ is provided.
b. The tissue cell permeation test device 200 is provided, including oral or intestinal membrane cells 280 cells grown on the well insert 250 floating in the larger well 210 over suitable cell culture media serum 230 contained in the well 210.
c. The first liquid composition is applied to the donor chamber 240, and allowed to diffuse across the membrane cells 280.
d. Samples containing the cannabinoid profile 10′ having diffused across the membrane cells 280 are then taken over a time course from the cell culture media serum 230 in the receptor chamber 220. The end cannabinoid concentration [C]_{E} is measured in each of the taken samples to generate a concentration versus time curve [C]_{E}/T.
e. The same procedure can be applied to a second liquid composition containing the second portion of the cannabinoid profile.

Note that for the purpose of the present description, the above defined test procedure will be referred to as “Tissue cell permeation test”.

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

As used herein, the term “absorption” means the net movement of a substance from the site of administration (e.g., oral cavity, the gastrointestinal (GD tract and skin) to the bloodstream. Factors that affect absorption may include, but are not limited to, the solubility of a substance in the GI environment and the permeability of a substance through the GI membrane.

As used herein, the term “t.” or “time of peak concentration” is generally understood as the time for a compound to reach peak plasma concentration after the compound is administered into the body of a subject. Peak plasma concentration is the point of maximum concentration of a compound in the plasma after administration of the compound. The tₘₐₓ represents the time when the rate of absorption equals the rate of elimination of the compound and is an indicator of a compound's bioavailability.

As used herein, a cannabinoid is psychoactive if it affects mood, perception, consciousness, cognition or behaviour of a subject when consumed, as a result of changes in the functioning of the nervous system. Psychoactive effects of a cannabinoid may include euphoria, enhanced well-being, easy laughter, relaxation, fatigue, sleepiness, dysphoria, anxiety, panic, paranoia, depersonalisation, increased sensory perception, feeling of the body floating or sinking, heightened sexual experience, hallucinations, alteration of time perception, aggravation of psychotic states, fragmented thinking, enhanced creativity, disturbed memory, difficulty in concentration, headache, unsteady gait, ataxia, slurred speech, weakness, deterioration or amelioration of motor coordination, impaired learning, analgesia, muscle relaxation, improved taste responsiveness, appetite stimulation, cravings for cannabis, nausea, vomiting, and antiemetic effects.

As used herein, a “cannabis derived compound” refers to any compound which can be extracted from a cannabis plant material, such as a cannabinoid, a terpene, a flavonoid, and the like.

As used herein, a fast onset may reflect the case where the tₘₐₓ of the cannabinoid in a subject having ingested an edible or liquid composition (such as beverages) herein described composition is significantly faster than with conventional cannabis-infused edibles or beverages.

For example, a fast onset may be characterized as a tₘₐₓ of the cannabinoid in a subject having ingested the edible or liquid composition (such as beverages) within the range of from about 15 minutes to about 1 hour 45 minutes, or from about 15 minutes to about 1 hour 30 minutes, or from about 15 minutes to about 1 hour 15 minutes, or from about 15 minutes to about 1 hour, or from about 15 minutes to about 45 minutes, or from about 15 minutes to about 30 minutes, including any values therein.

As used herein, the controlled offset may reflect the case where the tₘₐₓ of the cannabinoid in a subject having ingested an edible or liquid composition (such as beverages) comprising the herein described composition is significantly decreases by at least about 50% (e.g., 50%, 55%, 60%, 65%, 70%, 75%, 80%, 85%, 90%, 95% or any value therein) in less than about 3 hours from the time of tₘₐₓ such as for example in less than about 2 hours 30 minutes from the time of tₘₐₓ or in less than about 2 hours 15 minutes from the time of tₘₐₓ in less than about 2 hours from the time of tₘₐₓ or in less than about 1 hour 45 minutes from the time of tₘₐₓ, or in less than about 1 hour 30 minutes from the time of tₘₐₓ or in less than about 1 hour 15 minutes from the time of tₘₐₓ or in less than about 1 hour from the time of tₘₐₓ or in less than about 45 minutes from the time of tₘₐₓ or in less than about 30 minutes from the time of tₘₐₓ.

As used herein, the term “carrier oil” is generally understood to but are not limited to, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (e.g. caprylic/capric glycerides or MCT), and any combination thereof.

As used herein, an encapsulating agent is generally understood to be natural or synthetic biopolymers, including proteins, carbohydrates, lipids, fats, and gums, or one or more small-molecule surfactants, or any combination thereof. In some embodiments, the one or more encapsulating agents may be gum arabic; starches such as corn starch; modified starches such as octenyl succinate modified starches; modified cellulose such as methyl cellulose, hydroxypropyl cellulose, methyl hydroxypropyl cellulose, and carboxymethylcellulose; certain types of pectin such as beet pectin; polysaccharides such as maltodextrin and soy soluble polysaccharides; corn fiber gum; globular proteins such as whey protein and whey protein ingredients such as whey protein concentrate, whey protein isolate, and highly purified protein fractions such as β-lactoglobulin and α-lactalbumin; flexible proteins such as gelatin and caseins such as sodium caseinate, calcium caseinate, and purified protein fractions such as β-casein; Tweens™ (polysorbates) such as Tween 20 (polyoxyethylene sorbitan monolaurate), Tween 40 (polyoxyethylene sorbitan monopalmitate), Tween 60 (polyoxyethylene sorbitan monostearate), Tween 40 (polyoxyethylene sorbitan monopalmitate), Tween 60 (polyoxyethylene sorbitan monostearate), and Tween 80 (polyoxyethylene sorbitan monooleate); sugar esters such as sucrose monopalmitate, sucrose monostearate, sucrose distearate, sucrose polystearate, and sucrose laurate; quillaja saponin (Q-Naturale™ and components thereof; sorbitan esters (Spans™) such as Span 20 (sorbitan monolaurate), Span 40 (sorbitan monopalmitate), Span 60 (sorbitan monostearate), Span 80 (sorbitan monooleate); amphiphilic block copolymers; cholesterol; egg yolk- and soy-derived phosphatidylcholines; cyclodextrins such as 2-hydroxypropyl-β-cyclodextrin; lecithin; or any combination thereof.

A mucolytic is generally understood to include any compound or agent that, when added to a liquid formulation, improves the permeation of the liquid formulation or the cannabinoid contained therein across the mucus membrane, and enhances the absorption of the formulation or the cannabinoid contained therein into the body.

Examples of mucolytics include, but are not limited to, papain, bromelain, trypsin, chymotrypsin, pepsin, protease, proteinase K, bromelain-palmitate, papain-palmitate, trypsin-palmitate, N-acetylcysteine, Pluronic F-127, N-dodecyl-4-mercaptobutanimidamide, and 2-mercapto-N-octylacetamide.

In the context of the present disclosure, an efflux blocker is any compound that inhibits efflux transporters and decreases the elimination of the cannabinoid or the microencapsulation composition containing the cannabinoid from the body. An efflux transporter is a cell-membrane transporter that pumps compounds out of a cell to eliminate such compounds from the body. Efflux transporters are located on all cell membranes but are more concentrated on membranes of cells in the gastrointestinal tract, liver and kidney.

Examples of efflux blockers include, but are not limited to, piperine, epigallocatechin gallate, 8-prenylnaringenin, icaritin, baicalein, biochanin A, silymarin, kaempferol, naringenin, quercetin, procyanidine, 3,5,7,3,4-pentamethoxyflavone, 5,7-dimethoxyflavone, myricetin, wogonin, resveratrol, genistein, chalcone, silymarin, phloretin, morin, (±)-praeruptorin A, (±)-30-O,40-O-dicynnamoyl-cis-khellactone, decursinol, farnesiferol A, galbanic acid, driportlandin, dihydroxybergamotin, bergamotin, bergaptol, bergapten, cnidiadin, dihydro-b-agarofuransesquiterpene, obacunone, uphoractine, pepluane, paraliane, latilagascenes B-I, tuckeyanols A-B, euphotuckeyanol, euphodendroidin D, pepluanin A, euphocharacin, euphomelliferine, euphomelliferine A, helioscopinolide A, B, E, and F, euphoportlandol A, euphoportlandol B, glaucine, lobeline, cepharanthine, 6b-benzoyloxy-3R-(Z)-(3,4,5-trimethoxycinnamoyloxy)tropane, 6b-benzoyloxy-3a-(3,4,5-trimethoxycinnamoyloxy)tropane, 6b-benzoyloxy-3a-(E)-(3,4,5-trimethoxycinnamoyloxy)tropane-7b-ol, 7b-acetoxy-6b-benzoyloxy-3a-(E)-(3,4,5-trimethoxycinnamoyloxy)tropane, pervilleines B-C, veralosinine, veranigrine, kopsiflorine, kopsamine, pleiocarpine, 11-methoxykopsilongine, lahadinine A, N-methoxycarbonyl-11, 12-methylenedioxy-D-16,17-kopsinine, 3-O-Rha(1-2)[Ara(1-4)]Glc-pennogenine, gracillin, polyphyllin D, 20-hydroxyecdysone, pinnatasterone, balsaminagenin B, balsaminoside A, karavelagenin C, protopanaxatriolginsenoside, tenacissimoside A, 11R-O-benzoyl-12-O-acetyltenacigenin B, astragaloside II, taccalonolides A,E,B, and N, primulanin, ardisimamilloside, diltiazem, bepridil, nicardipine, nifedipine, felodipine, isradipine, trifluorperazine, clopenthixol, trifluopromazine, flupenthixol, chlorpromazine, prochlorperazine, quinine, dexverapamil, emopamil, gallopamil, zosuquidar, elacridar, biricodar, timcodar, tariquidar, verapamil, cyclosporine A, reserpine, quinidine, yohimbine, tamoxifen, toremifene, dexverapamil, dexniguldipine, valspodar, dofequidar fumarate, cyclopropyldibenzosuberane zosuquidar, laniquidar, and mitotane.

As used herein, the term “beverage base” means the water, juice or dairy base to which other ingredients may be added to make up the liquid beverage product. For example, for flavored sodas carbonated water and flavorings may serve as the beverage base to which the cannabinoid concentrate composition may be added. The beverage base may contain other ingredients such as, for non-limiting example, preservatives, flavorants, sweeteners, stabilizers, dyes, or carbonation. Preferably, the beverage base is a cannabinoid-less beverage.

As used herein, the expression “substantially the same” as used herein when referring to a tested parameter of a cannabis-infused product when compared to the same parameter tested in the base product generally refers to the value resulting from the test being more or less 20%, identical, or more or less 15% identical, or more or less 10% identical. Typically, such will occur when a sensory evaluation (by a subject, e.g., tasting, smelling, looking, touching) will not detect any significant variations and yet, depending on the instrumentation used, may result in slight measured variations, e.g., more or less 20%, identical, or more or less 15% identical, or more or less 10% identical. However, because it is the sensory evaluation which likely has a more significant effect on the user experience and/or derived commercial benefit, even such slight variations will be deemed to be “substantially the same” for the purposes of the user's perspective, i.e., the consumer.

As used herein, the term “nanoemulsion” means an emulsion which is mainly constituted of particles having a particle size distribution which is less than about 1000 nm. In other words, the emulsion is made of at least 95%, or at least 96%, or at least 97%, or at least 98%, or at least 99% of particles in the nanometric range (i.e., from 0 to 1000 nm).

The term “particle size”, as used herein, refers to a volume based particle size measured, for example, by laser diffraction method. Laser diffraction measures particle size distribution by measuring the angular variation in intensity of light scattered as a laser beam passes through a dispersed particulate sample. Large particles scatter light at small angles relative to the laser beam and small particles scatter light at large angles. The angular scattering intensity data is then analyzed to calculate the size of the particles responsible for creating the scattering pattern, for example, using the Mie theory of light scattering. The particle size is reported as a volume equivalent sphere diameter. Alternatively, the PSD can be measured by laser diffraction according to ISO 13320:2009 and ISO 9276-2:2014.

The following clauses provide a further description of examples of methods and compositions in accordance with the present disclosure:

### Clause Set 1:

Clause 1: A liquid formulation comprising a cannabinoid and an agent that modulates the absorption of the cannabinoid, wherein the tₘₐₓ of the cannabinoid in a subject who has consumed the liquid formulation is within the range of from about 15 minutes to about 2 hours, from about 15 minutes to about 1 hour 45 minutes, from about 15 minutes to about 1 hour 30 minutes, from about 15 minutes to about 1 hour 15 minutes, from about 15 minutes to about 1 hour, from about 15 minutes to about 45 minutes, from about 15 minutes to about 30 minutes, from about 30 minutes to about 2 hours, from about 30 minutes to about 1 hour 45 minutes, from about 30 minutes to about 1 hour 30 minutes, from about 30 minutes to about 1 hour 15 minutes, from about 30 minutes to about 1 hour, from about 30 minutes to about 45 minutes, from about 45 minutes to about 2 hours, from about 45 minutes to about 1 hour 45 minutes, from about 45 minutes to about 1 hour 30 minutes, from about 45 minutes to about 1 hour 15 minutes, from about 45 minutes to about 1 hour, from about 1 hour to about 2 hours, from about 1 hour to about 1 hour 45 minutes, from about 1 hour to about 1 hour 30 minutes, from about 1 hour to about 1 hour 15 minutes, from about 1 hour 15 minutes to about 2 hours, from about 1 hour 15 minutes to about 1 hour 45 minutes, from about 1 hour 15 minutes to about 1 hour 30 minutes, from about 1 hour 30 minutes to about 2 hours, from about 1 hour 30 minutes to about 1 hour 45 minutes, or from about 1 hour 45 minutes to about 2 hours.

Clause 2: A liquid formulation comprising a cannabinoid and an agent that modulates the absorption of the cannabinoid, wherein the blood concentration of the cannabinoid in a subject who has consumed the liquid formulation decreases by at least 50% in less than about 3 hours from the time of tₘₐₓ at least about 50% in less than about 2 hours 45 minutes from the time of tₘₐₓ at least about 50% in less than about 2 hours 30 minutes from the time of tₘₐₓ at least about 50% in less than about 2 hours 15 minutes from the time of tₘₐₓ, at least about 50% in less than about 2 hours from the time of tₘₐₓ at least about 50% in less than about 1 hour 45 minutes from the time of tₘₐₓ at least about 50% in less than about 1 hour 30 minutes from the time of tₘₐₓ at least about 50% in less than about 1 hour 15 minutes from the time of tₘₐₓ at least about 50% in less than about 1 hour from the time of tₘₐₓ at least about 50% in less than about 45 minutes from the time of tₘₐₓ at least about 50% in less than about 30 minutes from the time of tₘₐₓ at least about 55% in less than about 3 hours from the time of tₘₐₓ at least about 55% in less than about 2 hours 45 minutes from the time of tₘₐₓ at least about 55% in less than about 2 hours 30 minutes from the time of tₘₐₓ at least about 55% in less than about 2 hours 15 minutes from the time of tₘₐₓ at least about 55% in less than about 2 hours from the time of tₘₐₓ at least about 55% in less than about 1 hour 45 minutes from the time of tₘₐₓ, at least about 55% in less than about 1 hour 30 minutes from the time of tₘₐₓ, at least about 55% in less than about 1 hour 15 minutes from the time of tₘₐₓ at least about 55% in less than about 1 hour from the time of tₘₐₓ, at least about 55% in less than about 45 minutes from the time of tₘₐₓ, at least about 55% in less than about 30 minutes from the time of tₘₐₓ, at least about 60% in less than about 3 hours from the time of tₘₐₓ, at least about 60% in less than about 2 hours 45 minutes from the time of tₘₐₓ, at least about 60% in less than about 2 hours 30 minutes from the time of tₘₐₓ, at least about 60% in less than about 2 hours 15 minutes from the time of tₘₐₓ, at least about 60% in less than about 2 hours from the time of tₘₐₓ, at least about 60% in less than about 1 hour 45 minutes from the time of tₘₐₓ, at least about 60% in less than about 1 hour 30 minutes from the time of tₘₐₓ, at least about 60% in less than about 1 hour 15 minutes from the time of tₘₐₓ, at least about 60% in less than about 1 hour from the time of tₘₐₓ, at least about 60% in less than about 45 minutes from the time of tₘₐₓ, at least about 60% in less than about 30 minutes from the time of tₘₐₓ, at least about 65% in less than about 3 hours from the time of tₘₐₓ, at least about 65% in less than about 2 hours 45 minutes from the time of tₘₐₓ, at least about 65% in less than about 2 hours 30 minutes from the time of tₘₐₓ, by at least about 65% in less than about 2 hours 15 minutes from the time of tₘₐₓ, by at least about 65% in less than about 2 hours from the time of tₘₐₓ, at least about 65% in less than about 1 hour 45 minutes from the time of tₘₐₓ, at least about 65% in less than about 1 hour 30 minutes from the time of tₘₐₓ, at least about 65% in less than about 1 hour 15 minutes from the time of tₘₐₓ, at least about 65% in less than about 1 hour from the time of tₘₐₓ, at least about 65% in less than about 45 minutes from the time of tₘₐₓ, at least about 65% in less than about 30 minutes from the time of tₘₐₓ, at least about 70% in less than about 3 hours from the time of tₘₐₓ, at least about 70% in less than about 2 hours 45 minutes from the time of tₘₐₓ, at least about 70% in less than about 2 hours 30 minutes from the time of tₘₐₓ, at least about 70% in less than about 2 hours 15 minutes from the time of tₘₐₓ, at least about 70% in less than about 2 hours from the time of tₘₐₓ, at least about 70% in less than about 1 hour 45 minutes from the time of tₘₐₓ, at least about 70% in less than about 1 hour 30 minutes from the time of tₘₐₓ, at least about 70% in less than about 1 hour 15 minutes from the time of tₘₐₓ, at least about 70% in less than about 1 hour from the time of tₘₐₓ, at least about 70% in less than about 45 minutes from the time of tₘₐₓ, at least about 70% in less than about 30 minutes from the time of tₘₐₓ, at least about 75% in less than about 3 hours from the time of tₘₐₓ, at least about 75% in less than about 2 hours 45 minutes from the time of tₘₐₓ, at least about 75% in less than about 2 hours 30 minutes from the time of tₘₐₓ, at least about 75% in less than about 2 hours 15 minutes from the time of tₘₐₓ, at least about 75% in less than about 2 hours from the time of tₘₐₓ, at least about 75% in less than about 1 hour 45 minutes from the time of tₘₐₓ, at least about 75% in less than about 1 hour 30 minutes from the time of tₘₐₓ, at least about 75% in less than about 1 hour 15 minutes from the time of tₘₐₓ, at least about 75% in less than about 1 hour from the time of tₘₐₓ, at least about 75% in less than about 45 minutes from the time of tₘₐₓ, at least about 75% in less than about 30 minutes from the time of tₘₐₓ, at least about 80% in less than about 3 hours from the time of tₘₐₓ, at least about 80% in less than about 2 hours 45 minutes from the time of tₘₐₓ, at least about 80% in less than about 2 hours 30 minutes from the time of tₘₐₓ, at least about 80% in less than about 2 hours 15 minutes from the time of tₘₐₓ, at least about 80% in less than about 2 hours from the time of tₘₐₓ, at least about 80% in less than about 1 hour 45 minutes from the time of tₘₐₓ, at least about 80% in less than about 1 hour 30 minutes from the time of tₘₐₓ, at least about 80% in less than about 1 hour 15 minutes from the time of tₘₐₓ, at least about 80% in less than about 1 hour from the time of tₘₐₓ, at least about 80% in less than about 45 minutes from the time of tₘₐₓ, at least about 80% in less than about 30 minutes from the time of tₘₐₓ, at least about 85% in less than about 3 hours from the time of tₘₐₓ, at least about 85% in less than about 2 hours 45 minutes from the time of tₘₐₓ, at least about 85% in less than about 2 hours 30 minutes from the time of tₘₐₓ, at least about 85% in less than about 2 hours 15 minutes from the time of tₘₐₓ, at least about 85% in less than about 2 hours from the time of tₘₐₓ, at least about 85% in less than about 1 hour 45 minutes from the time of tₘₐₓ, at least about 85% in less than about 1 hour 30 minutes from the time of tₘₐₓ, at least about 85% in less than about 1 hour 15 minutes from the time of tₘₐₓ, at least about 85% in less than about 1 hour from the time of tₘₐₓ, at least about 85% in less than about 45 minutes from the time of tₘₐₓ, at least about 85% in less than about 30 minutes from the time of tₘₐₓ, at least about 90% in less than about 3 hours from the time of tₘₐₓ, at least about 90% in less than about 2 hours 45 minutes from the time of tₘₐₓ, at least about 90% in less than about 2 hours 30 minutes from the time of tₘₐₓ, at least about 90% in less than about 2 hours 15 minutes from the time of tₘₐₓ, at least about 90% in less than about 2 hours from the time of tₘₐₓ, at least about 90% in less than about 1 hour 45 minutes from the time of tₘₐₓ, at least about 90% in less than about 1 hour 30 minutes from the time of tₘₐₓ, at least about 90% in less than about 1 hour 15 minutes from the time of tₘₐₓ, at least about 90% in less than about 1 hour from the time of tₘₐₓ, at least about 90% in less than about 45 minutes from the time of tₘₐₓ, at least about 90% in less than about 30 minutes from the time of tₘₐₓ, at least about 95% in less than about 3 hours from the time of tₘₐₓ, at least about 95% in less than about 2 hours 45 minutes from the time of tₘₐₓ, at least about 95% in less than about 2 hours 30 minutes from the time of tₘₐₓ, at least about 95% in less than about 2 hours 15 minutes from the time of tₘₐₓ, at least about 95% in less than about 2 hours from the time of tₘₐₓ, at least about 95% in less than about 1 hour 45 minutes from the time of tₘₐₓ, at least about 95% in less than about 1 hour 30 minutes from the time of tₘₐₓ, at least about 95% in less than about 1 hour 15 minutes from the time of tₘₐₓ, at least about 95% in less than about 1 hour from the time of tₘₐₓ, at least about 95% in less than about 45 minutes from the time of tₘₐₓ, or at least about 95% in less than about 30 minutes from the time of tₘₐₓ.

Clause 3: A liquid formulation comprising a cannabinoid and an agent that modulates the absorption of the cannabinoid, wherein the blood concentration of the cannabinoid in a subject who has consumed the liquid formulation is no more than about 10 ng/mL in less than about 3 hours from the time of tₘₐₓ, no more than about 10 ng/mL in less than about 2 hour 45 minutes from the time of tₘₐₓ, no more than about 10 ng/mL in less than about 2 hour 30 minutes from the time of tₘₐₓ, no more than about 10 ng/mL in less than about 2 hour 15 minutes from the time of tₘₐₓ, no more than about 10 ng/mL in less than about 2 hours from the time of tₘₐₓ, no more than about 10 ng/mL in less than about 1 hour and 45 minutes from the time of tₘₐₓ, no more than about 10 ng/mL in less than about 1 hour 30 minutes from the time of tₘₐₓ, no more than about 10 ng/mL in less than about 1 hour 15 minutes from the time of tₘₐₓ, no more than about 10 ng/mL in less than about 1 hour from the time of tₘₐₓ, no more than about 10 ng/mL in less than about 45 minutes from the time of tₘₐₓ, no more than about 10 ng/mL in less than about 30 minutes from the time of tₘₐₓ, no more than about 9 ng/mL in less than about 3 hours from the time of tₘₐₓ, no more than about 9 ng/mL in less than about 2 hour 45 minutes from the time of tₘₐₓ, no more than about 9 ng/mL in less than about 2 hour 30 minutes from the time of tₘₐₓ, no more than about 9 ng/mL in less than about 2 hour 15 minutes from the time of tₘₐₓ, no more than about 9 ng/mL in less than about 2 hours from the time of tₘₐₓ, no more than about 9 ng/mL in less than about 1 hour and 45 minutes from the time of tₘₐₓ, no more than about 9 ng/mL in less than about 1 hour 30 minutes from the time of tₘₐₓ, no more than about 9 ng/mL in less than about 1 hour 15 minutes from the time of tₘₐₓ, no more than about 9 ng/mL in less than about 1 hour from the time of tₘₐₓ, no more than about 9 ng/mL in less than about 45 minutes from the time of tₘₐₓ, no more than about 9 ng/mL in less than about 30 minutes from the time of tₘₐₓ, no more than about 8 ng/mL in less than about 3 hours from the time of tₘₐₓ, no more than about 8 ng/mL in less than about 2 hour 45 minutes from the time of tₘₐₓ, no more than about 8 ng/mL in less than about 2 hour 30 minutes from the time of tₘₐₓ, no more than about 8 ng/mL in less than about 2 hour 15 minutes from the time of tₘₐₓ, no more than about 8 ng/mL in less than about 2 hours from the time of tₘₐₓ, no more than about 8 ng/mL in less than about 1 hour and 45 minutes from the time of tₘₐₓ, no more than about 8 ng/mL in less than about 1 hour 30 minutes from the time of tₘₐₓ, no more than about 8 ng/mL in less than about 1 hour 15 minutes from the time of tₘₐₓ, no more than about 8 ng/mL in less than about 1 hour from the time of tₘₐₓ, no more than about 8 ng/mL in less than about 45 minutes from the time of tₘₐₓ, no more than about 8 ng/mL in less than about 30 minutes from the time of tₘₐₓ, no more than about 7 ng/mL in less than about 3 hours from the time of tₘₐₓ, no more than about 7 ng/mL in less than about 2 hour 45 minutes from the time of tₘₐₓ, no more than about 7 ng/mL in less than about 2 hour 30 minutes from the time of tₘₐₓ, no more than about 7 ng/mL in less than about 2 hour 15 minutes from the time of tₘₐₓ, no more than about 7 ng/mL in less than about 2 hours from the time of tₘₐₓ, no more than about 7 ng/mL in less than about 1 hour and 45 minutes from the time of tₘₐₓ, no more than about 7 ng/mL in less than about 1 hour 30 minutes from the time of tₘₐₓ, no more than about 7 ng/mL in less than about 1 hour 15 minutes from the time of tₘₐₓ, no more than about 7 ng/mL in less than about 1 hour from the time of tₘₐₓ, no more than about 7 ng/mL in less than about 45 minutes from the time of tₘₐₓ, no more than about 7 ng/mL in less than about 30 minutes from the time of tₘₐₓ, no more than about 6 ng/mL in less than about 3 hours from the time of tₘₐₓ, no more than about 6 ng/mL in less than about 2 hour 45 minutes from the time of tₘₐₓ, no more than about 6 ng/mL in less than about 2 hour 30 minutes from the time of tₘₐₓ, no more than about 6 ng/mL in less than about 2 hour 15 minutes from the time of tₘₐₓ, no more than about 6 ng/mL in less than about 2 hours from the time of tₘₐₓ, no more than about 6 ng/mL in less than about 1 hour and 45 minutes from the time of tₘₐₓ, no more than about 6 ng/mL in less than about 1 hour 30 minutes from the time of tₘₐₓ, no more than about 6 ng/mL in less than about 1 hour 15 minutes from the time of tₘₐₓ, no more than about 6 ng/mL in less than about 1 hour from the time of tₘₐₓ, no more than about 6 ng/mL in less than about 45 minutes from the time of tₘₐₓ, no more than about 6 ng/mL in less than about 30 minutes from the time of tₘₐₓ, no more than about 5 ng/mL in less than about 3 hours from the time of tₘₐₓ, no more than about 5 ng/mL in less than about 2 hour 45 minutes from the time of tₘₐₓ, no more than about 5 ng/mL in less than about 2 hour 30 minutes from the time of tₘₐₓ, no more than about 5 ng/mL in less than about 2 hour 15 minutes from the time of tₘₐₓ, no more than about 5 ng/mL in less than about 2 hours from the time of tₘₐₓ, no more than about 5 ng/mL in less than about 1 hour and 45 minutes from the time of tₘₐₓ, no more than about 5 ng/mL in less than about 1 hour 30 minutes from the time of tₘₐₓ, no more than about 5 ng/mL in less than about 1 hour 15 minutes from the time of tₘₐₓ, no more than about 5 ng/mL in less than about 1 hour from the time of tₘₐₓ, no more than about 5 ng/mL in less than about 45 minutes from the time of tₘₐₓ, no more than about 5 ng/mL in less than about 30 minutes from the time of tₘₐₓ, no more than about 4 ng/mL in less than about 3 hours from the time of tₘₐₓ, no more than about 4 ng/mL in less than about 2 hour 45 minutes from the time of tₘₐₓ, no more than about 4 ng/mL in less than about 2 hour 30 minutes from the time of tₘₐₓ, no more than about 4 ng/mL in less than about 2 hour 15 minutes from the time of tₘₐₓ, no more than about 4 ng/mL in less than about 2 hours from the time of tₘₐₓ, no more than about 4 ng/mL in less than about 1 hour and 45 minutes from the time of tₘₐₓ, no more than about 4 ng/mL in less than about 1 hour 30 minutes from the time of tₘₐₓ, no more than about 4 ng/mL in less than about 1 hour 15 minutes from the time of tₘₐₓ, no more than about 4 ng/mL in less than about 1 hour from the time of tₘₐₓ, no more than about 4 ng/mL in less than about 45 minutes from the time of tₘₐₓ, no more than about 4 ng/mL in less than about 30 minutes from the time of tₘₐₓ, no more than about 3 ng/mL in less than about 3 hours from the time of tₘₐₓ, no more than about 3 ng/mL in less than about 2 hour 45 minutes from the time of tₘₐₓ, no more than about 3 ng/mL in less than about 2 hour 30 minutes from the time of tₘₐₓ, no more than about 3 ng/mL in less than about 2 hour 15 minutes from the time of tₘₐₓ, no more than about 3 ng/mL in less than about 2 hours from the time of tₘₐₓ, no more than about 3 ng/mL in less than about 1 hour and 45 minutes from the time of tₘₐₓ, no more than about 3 ng/mL in less than about 1 hour 30 minutes from the time of tₘₐₓ, no more than about 3 ng/mL in less than about 1 hour 15 minutes from the time of tₘₐₓ, no more than about 3 ng/mL in less than about 1 hour from the time of tₘₐₓ, no more than about 3 ng/mL in less than about 45 minutes from the time of tₘₐₓ, no more than about 3 ng/mL in less than about 30 minutes from the time of tₘₐₓ, no more than about 2 ng/mL in less than about 3 hours from the time of tₘₐₓ, no more than about 2 ng/mL in less than about 2 hour 45 minutes from the time of tₘₐₓ, no more than about 2 ng/mL in less than about 2 hour 30 minutes from the time of tₘₐₓ, no more than about 2 ng/mL in less than about 2 hour 15 minutes from the time of tₘₐₓ, no more than about 2 ng/mL in less than about 2 hours from the time of tₘₐₓ, no more than about 2 ng/mL in less than about 1 hour and 45 minutes from the time of tₘₐₓ, no more than about 2 ng/mL in less than about 1 hour 30 minutes from the time of tₘₐₓ, no more than about 2 ng/mL in less than about 1 hour 15 minutes from the time of tₘₐₓ, no more than about 2 ng/mL in less than about 1 hour from the time of tₘₐₓ, no more than about 2 ng/mL in less than about 45 minutes from the time of tₘₐₓ, no more than about 2 ng/mL in less than about 30 minutes from the time of tₘₐₓ, no more than about 1 ng/mL in less than about 3 hours from the time of tₘₐₓ, no more than about 1 ng/mL in less than about 2 hour 45 minutes from the time of tₘₐₓ, no more than about 1 ng/mL in less than about 2 hour 30 minutes from the time of tₘₐₓ, no more than about 1 ng/mL in less than about 2 hour 15 minutes from the time of tₘₐₓ, no more than about 1 ng/mL in less than about 2 hours from the time of tₘₐₓ, no more than about 1 ng/mL in less than about 1 hour and 45 minutes from the time of tₘₐₓ, no more than about 1 ng/mL in less than about 1 hour 30 minutes from the time of tₘₐₓ, no more than about 1 ng/mL in less than about 1 hour 15 minutes from the time of tₘₐₓ, no more than about 1 ng/mL in less than about 1 hour from the time of tₘₐₓ, no more than about 1 ng/mL in less than about 45 minutes from the time of tₘₐₓ, no more than about 1 ng/mL in less than about 30 minutes from the time of tₘₐₓ.

Clause 4: The liquid formulation of Clause 2, wherein the tₘₐₓ of the cannabinoid in a subject who has consumed the liquid formulation is within the range of from about 15 minutes to about 2 hours, from about 15 minutes to about 1 hour 45 minutes, from about 15 minutes to about 1 hour 30 minutes, from about 15 minutes to about 1 hour 15 minutes, from about 15 minutes to about 1 hour, from about 15 minutes to about 45 minutes, from about 15 minutes to about 30 minutes, from about 30 minutes to about 2 hours, from about 30 minutes to about 1 hour 45 minutes, from about 30 minutes to about 1 hour 30 minutes, from about 30 minutes to about 1 hour 15 minutes, from about 30 minutes to about 1 hour, from about 30 minutes to about 45 minutes, from about 45 minutes to about 2 hours, from about 45 minutes to about 1 hour 45 minutes, from about 45 minutes to about 1 hour 30 minutes, from about 45 minutes to about 1 hour 15 minutes, from about 45 minutes to about 1 hour, from about 1 hour to about 2 hours, from about 1 hour to about 1 hour 45 minutes, from about 1 hour to about 1 hour 30 minutes, from about 1 hour to about 1 hour 15 minutes, from about 1 hour 15 minutes to about 2 hours, from about 1 hour 15 minutes to about 1 hour 45 minutes, from about 1 hour 15 minutes to about 1 hour 30 minutes, from about 1 hour 30 minutes to about 2 hours, from about 1 hour 30 minutes to about 1 hour 45 minutes, or from about 1 hour 45 minutes to about 2 hours.

Clause 5: The liquid formulation of Clause 3, wherein the tₘₐₓ of the cannabinoid in a subject who has consumed the liquid formulation is within the range of from about 15 minutes to about 2 hours, from about 15 minutes to about 1 hour 45 minutes, from about 15 minutes to about 1 hour 30 minutes, from about 15 minutes to about 1 hour 15 minutes, from about 15 minutes to about 1 hour, from about 15 minutes to about 45 minutes, from about 15 minutes to about 30 minutes, from about 30 minutes to about 2 hours, from about 30 minutes to about 1 hour 45 minutes, from about 30 minutes to about 1 hour 30 minutes, from about 30 minutes to about 1 hour 15 minutes, from about 30 minutes to about 1 hour, from about 30 minutes to about 45 minutes, from about 45 minutes to about 2 hours, from about 45 minutes to about 1 hour 45 minutes, from about 45 minutes to about 1 hour 30 minutes, from about 45 minutes to about 1 hour 15 minutes, from about 45 minutes to about 1 hour, from about 1 hour to about 2 hours, from about 1 hour to about 1 hour 45 minutes, from about 1 hour to about 1 hour 30 minutes, from about 1 hour to about 1 hour 15 minutes, from about 1 hour 15 minutes to about 2 hours, from about 1 hour 15 minutes to about 1 hour 45 minutes, from about 1 hour 15 minutes to about 1 hour 30 minutes, from about 1 hour 30 minutes to about 2 hours, from about 1 hour 30 minutes to about 1 hour 45 minutes, or from about 1 hour 45 minutes to about 2 hours.

Clause 6: The liquid formulation of any one of Clauses 1 to 5, wherein the liquid formulation is a drink.

Clause 7: The liquid formulation of Clause 6, wherein the liquid formulation is drinking water, milk (both diary and non-diary), juice, a smoothie, coffee or a caffeinated beverage, tea, herbal tea, a cocoa beverage, a carbonated drink, a nitrogenated drink, an energy drink, a fermented beverage, or an alcoholic beverage.

Clause 8: The liquid formulation of Clause 7, wherein the liquid formulation is a carbonated drink or a nitrogenated drink.

Clause 9: The liquid formulation of Clause 8, wherein the liquid formulation has zero calories.

Clause 10: The liquid formulation of Clause 7, wherein the liquid formulation is an alcoholic drink, such as beer, lager, cider, spirit, wine/fortified wine, and cocktail.

Clause 11: The liquid formulation of any one of Clauses 1 to 10, wherein the cannabinoid is tetrahydrocannabinol (THC).

Clause 12: The liquid formulation of any one of Clauses 1 to 10, wherein the cannabinoid is cannabidiol (CBD).

Clause 13: The liquid formulation of any one of Clauses 1 to 10, wherein the cannabinoid is a mixture of tetrahydrocannabinol (THC) and cannabidiol (CBD).

Clause 14: The liquid formulation of Clause 13, wherein the ratio of THC to CBD in the liquid formulation is about 1:1.

Clause 15: The liquid formulation of any one of Clauses 1 to 14, wherein the subject is a human.

Clause 16: The liquid formulation of any one of Clauses 1 to 14, wherein the subject is an animal.

Clause 17: The liquid formulation of Clause 16, wherein the animal is a canine or a feline.

Clause 18: The liquid formulation of any one of Clauses 1 to 17, wherein the liquid formulation is clear.

Clause 19: The liquid formulation of Clause 18, wherein the liquid formulation has a turbidity of less than 0.05 cm⁻¹ at 600 nm.

Clause 20: The liquid formulation of any one of Clauses 1 to 19, wherein the liquid formulation does not have a disagreeable taste.

Clause 21: The liquid formulation of any one of Clauses 1 to 20, wherein the liquid formulation is stable for at least 1 month at 4° C.

Clause 22: The liquid formulation of any one of Clauses 1 to 20, wherein the liquid formulation is stable for at least 1 month at room temperature.

Clause 23: The liquid formulation of any one of Clauses 1 to 22, wherein the agent that modulates the absorption of the cannabinoid comprises an encapsulating agent that forms a microencapsulation system with the cannabinoid in the liquid formulation.

Clause 24: The liquid formulation of Clause 23, wherein the encapsulating agent is a film-forming natural or synthetic biopolymer, a small-molecule surfactant, or a combination thereof.

Clause 25: The liquid formulation of Clause 24, wherein the biopolymer is a protein, a carbohydrate, a lipid, a fat, or a gum.

Clause 26: The liquid formulation of Clause 24, wherein the encapsulating agent is gum arabic; starches such as corn starch; modified starches such as octenyl succinate modified starches; modified cellulose such as methyl cellulose, hydroxypropyl cellulose, methyl hydroxypropyl cellulose, and carboxymethylcellulose; certain types of pectin such as beet pectin; polysaccharides such as maltodextrin and soy soluble polysaccharides; corn fiber gum; globular proteins such as whey protein and whey protein ingredients such as whey protein concentrate, whey protein isolate, and highly purified protein fractions such as β-lactoglobulin and α-lactalbumin; flexible proteins such as gelatin and caseins such as sodium caseinate, calcium caseinate, and purified protein fractions such as β-casein; Tweens® (polysorbates) such as Tween (polyoxyethylene sorbitan monolaurate), Tween 40 (polyoxyethylene sorbitan monopalmitate), Tween 60 (polyoxyethylene sorbitan monostearate), Tween 40 (polyoxyethylene sorbitan monopalmitate), Tween 60 (polyoxyethylene sorbitan monostearate), and Tween 80 (polyoxyethylene sorbitan monooleate); sugar esters such as sucrose monopalmitate, sucrose monostearate, sucrose distearate, sucrose polystearate, and sucrose laurate; quillaja saponin (Q-Naturale®) and components thereof; sorbitan esters (Spans®) such as Span 20 (sorbitan monolaurate), Span 40 (sorbitan monopalmitate), Span 60 (sorbitan monostearate), Span 80 (sorbitan monooleate); amphiphilic block copolymers; cholesterol: egg yolk- and soy-derived phosphatidylcholines; cyclodextrnns such as 2-hydroxypropyl-β-cyclodextnn; lecithin; or any combination thereof.

Clause 27: The liquid formulation of any one of Clauses 23 to 26, wherein the microencapsulation system comprises emulsions, nanoemulsions, micelles, solid lipid nanoparticles, nanostructured lipid carriers, liposomes, nanoliposomes, niosomes, polymer particles, hydrogel particles, or combinations thereof.

Clause 28: The liquid formulation of Clause 27, wherein the microencapsulation system comprises emulsions and/or nanoemulsions.

Clause 29: The liquid formulation of Clause 28, wherein the encapsulating agent is an emulsifier, and the liquid formulation optionally further comprises at least one of a weighting agent, a ripening inhibitor, and a texture modifier.

Clause 30: The liquid formulation of Clause 29, wherein the emulsifier is a polysaccharide-based emulsifier, a protein-based emulsifier, a small-molecule surfactant, or a mixture thereof.

Clause 31: The liquid formulation of any one of Clauses 28 to 30, wherein the emulsions and/or nanoemulsions are prepared using a homogenizer.

Clause 32: The liquid formulation of any one of Clauses 28 to 30, wherein the emulsions and/or nanoemulsions are prepared using a spontaneous emulsification method, an emulsion inversion point method, and/or a phase inversion temperature method.

Clause 33: The liquid formulation of any one of Clauses 1 to 32, wherein the liquid formulation further comprises an antidote of the cannabinoid.

Clause 34: The liquid formulation of Clause 33, wherein the cannabinoid is THC, and the antidote of THC comprises at least one of CBD; Acorus calamus or an extract thereof; black pepper or an extract thereof; citrus or an extract thereof; pine nuts or an extract thereof; pistachio nuts or an extract thereof; fruits of Pistacia terbinthus or an extract thereof; piperine; and terpenes, such as β-caryophyllene, limonene, myrcene, and α-pinene.

Clause 35: The liquid formulation of Clause 34, wherein the antidote is encapsulated in a microencapsulation system that is different from the microencapsulation system of THC.

Clause 36: The liquid formulation of Clause 35, wherein the microencapsulation system of THC comprises particles having an average size of less than about 100 nm, and the microencapsulation system of the antidote comprises particles having an average size of more than about 100 nm.

Clause 37: The liquid formulation of any one of Clauses 23 to 36, wherein the liquid formulation is stored in a container comprising a de-emulsification agent that can be released into the liquid formulation.

Clause 38: The liquid formulation of Clause 37, wherein the de-emulsification agent is one or more acids, including, but not limited to, succinic acid, fumaric acid, and citric acid; bases, including, but not limited to, sodium carbonate, potassium carbonate, sodium hydroxide, and potassium hydroxide; alcohols, including, but not limited to, ethanol and glycerol; electrolytes, including, but not limited to, sodium sulfate, sodium chloride, and the aforementioned acids and bases; enzymes, including, but not limited to, cellulase, protease, amylase, and lipase; and the like.

Clause 39: The liquid formulation of any one of Clauses 1 to 37, wherein the agent that modulates the absorption of the cannabinoid comprises a mucolytic.

Clause 40: The liquid formulation of Clause 39, wherein the mucolytic comprises at least one of papain, bromelain, trypsin, chymotrypsin, pepsin, protease, proteinase K, bromelain-palmitate, papain-palmitate, trypsin-palmitate, N-acetylcysteine, Pluronic F-127, N-dodecyl-4-mercaptobutanimidamide, and 2-mercapto-N-octylacetamide.

Clause 41: The liquid formulation of any one of Clauses 1 to 40, wherein the agent that modulates the absorption of the cannabinoid comprises an efflux blocker.

Clause 42: The liquid formulation of Clause 41, wherein the efflux blocker comprises at least one of piperine, epigallocatechin gallate, 8-prenylnaringenin, icaritin, baicalein, Biochanin A, silymarin, kaempferol, naringenin, quercetin, procyanidine, 3,5,7,3,4-pentamethoxyflavone, 5,7-dimethoxyflavone, myricetin, wogonin, resveratrol, genistein, chalcone, silymarin, phloretin, morin, (±)-praeruptorin A, (±)-30-O,40-O-dicynnamoyl-cis-khellactone, decursinol, farnesiferol A, galbanic acid, driportlandin, dihydroxybergamotin, bergamotin, bergaptol, bergapten, cnidiadin, dihydro-b-agarofuransesquiterpene, obacunone, uphoractine, pepluane, paraliane, latilagascenes B-I, tuckeyanols A-B, euphotuckeyanol, euphodendroidin D, pepluanin A, euphocharacin, euphomelliferine, euphomelliferine A, helioscopinolide A, B, E, and F, euphoportlandol A, euphoportlandol B, glaucine, lobeline, cepharanthine, 6b-benzoyloxy-3R-(Z)-(3,4,5-trimethoxycinnamoyloxy)tropane, 6b-benzoyloxy-3a-(3,4,5-trimethoxycinnamoyl-oxy)tropane, 6b-benzoyloxy-3a-(E)-(3,4,5-trimethoxycinnamoyloxy)tropane-7b-ol, 7b-acetoxy-6b-benzoyloxy-3a-(E)-(3,4,5-trimethoxycinnamoyloxy)tropane, pervilleines B-C, veralosinine, veranigrine, kopsiflorine, kopsamine, pleiocarpine, 11-methoxykopsilongine, lahadinine A, N-methoxycarbonyl-11, 12-methylenedioxy-D-16,17-kops-inine, 3-O-Rha(1-2)[Ara(1-4)]Glc-pennogenine, gracillin, polyphyllin D, 20-hydroxyecdysone, pinnatasterone, balsaminagenin B, balsaminoside A, karavelagenin C, protopanaxatriolginsenoside, tenacissimoside A, 11R—O-benzoyl-12-O-acetyltenacigenin B, astragaloside II, taccalonolide A,E,B, and N, primulanin, ardisimamilloside, diltiazem, bepridil, nicardipine, nifedipine, felodipine, isradipine, trifluorperazine, clopenthixol, trifluopromazine, flupenthixol, chlorpromazine, prochlorperazine, quinine, dexverapamil, emopamil, gallopamil, zosuquidar, elacridar, biricodar, timcodar, tariquidar, verapamil, cyclosporine A, reserpine, quinidine, yohimbine, tamoxifen, toremifene, dexverapamil, dexniguldipine, valspodar, dofequidar fumarate, cyclopropyldibenzosuberane zosuquidar, laniquidar, and mitotane.

### Clause Set 2

Clause 1: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product having a turbidity of less than 0.05 cm−1 at 600 nm.

Clause 2: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product having a viscosity selected in the range of from 50 mPas to 1500 mPas.

Clause 3: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product having an odor index which is substantially the same as that one of the cannabinoid-less beverage.

Clause 4: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product having a tasting index which is substantially the same as that one of the cannabinoid-less beverage.

Clause 5: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product being stable for at least 1 month at 4° C.

Clause 6: A food additive comprising an emulsion of a cannabinoid which is at least partially miscible in water such that when the emulsion comprises 30 ml/ml of cannabinoids, dilution or infusion of the food additive in a cannabinoid-less beverage or blending with a beverage base results in a beverage product comprising at least 0.002 mg/ml of cannabinoid in volume of the beverage product.

Clause 7: The food additive of any one of Clauses 1 to 6, wherein the beverage product is selected from drinking water, dairy milk, non-dairy milk, juice, a smoothie, coffee or a caffeinated beverage, tea, herbal tea, an energy drink, a fermented beverage, non-alcoholic beer, and a cocoa beverage.

Clause 8: The food additive of any one of Clauses 1 to 6, wherein the beverage product is selected from a carbonated drink and a nitrogenated drink.

Clause 9: The food additive of any one of Clauses 1 to 6, wherein the beverage product is an alcoholic beverage.

Clause 10: The food additive of Clause 9, wherein the alcoholic beverage is selected from beer, lager, cider, spirit, wine/fortified wine, and cocktail.

Clause 11: The food additive of any one of Clauses 1 to 10, wherein the emulsion includes tetrahydrocannabinol (THC).

Clause 12: The food additive of any one of Clauses 1 to 10, wherein the emulsion includes cannabidiol (CBD).

Clause 13: The food additive of any one of Clauses 1 to 10, wherein the emulsion includes a terpene.

Clause 14: The food additive of any one of Clauses 1 to 10, wherein the emulsion includes tetrahydrocannabinol (THC) and cannabidiol (CBD).

Clause 15: The food additive of Clause 14, wherein the THC and CBD are present in a ratio of 1:1.

Clause 16: The food additive of any one of Clauses 1 to 15, in the form of a powder.

Clause 17: The food additive of any one of Clauses 1 to 15, in the form of a liquid.

Clause 18: The food additive of any one of Clauses 1 to 15, in the form of a capsule, lozenge or tablet.

Clause 19: The food additive of any one of Clauses 1 to 18, wherein the additive has less than 100,000 CFU of total viable aerobic bacteria count.

Clause 20: The food additive of any one of Clauses 1 to 19, wherein the additive has less than 100,000 CFU/g of total yeast and mold count, preferably less than 10,000 CFU/g.

Clause 21: The food additive of any one of Clauses 1 to 20, wherein the additive has less than 1000 CFU of bile-tolerant gram negative bacteria.

Clause 22: The food additive of any one of Clauses 1 to 21, wherein the additive has less than 1000 CFU/g of total coliforms count, preferably less than 100 CFU/g.

Clause 23: The food additive of any one of Clauses 1 to 22, comprising an encapsulating agent that forms a microencapsulation system with the cannabinoid.

Clause 24: The food additive of Clause 23, wherein the encapsulating agent is a film-forming natural or synthetic biopolymer, a small-molecule surfactant, or a combination thereof.

Clause 25: The food additive of Clause 24, wherein the biopolymer is a protein, a carbohydrate, a lipid, a fat, or a gum.

Clause 26: The food additive of Clause 24, wherein the encapsulating agent is arabic gum; starches such as corn starch; modified starches such as octenyl succinate modified starches; modified cellulose such as methyl cellulose, hydroxypropyl cellulose, methyl hydroxypropyl cellulose, and carboxymethylcellulose; certain types of pectin such as beet pectin; polysaccharides such as maltodextrin and soy soluble polysaccharides; corn fiber gum; globular proteins such as whey protein and whey protein ingredients such as whey protein concentrate, whey protein isolate, and highly purified protein fractions such as β-lactoglobulin and α-lactalbumin; flexible proteins such as gelatin and caseins such as sodium caseinate, calcium caseinate, and purified protein fractions such as β-casein; Tweens® (polysorbates) such as Tween 20 (polyoxyethylene sorbitan monolaurate), Tween 40 (polyoxyethylene sorbitan monopalmitate), Tween 60 (polyoxyethylene sorbitan monostearate), Tween 40 (polyoxyethylene sorbitan monopalmitate), Tween 60 (polyoxyethylene sorbitan monostearate), and Tween 80 (polyoxyethylene sorbitan monooleate); sugar esters such as sucrose monopalmitate, sucrose monostearate, sucrose distearate, sucrose polystearate, and sucrose laurate; quillaja saponin (Q-Naturale®) and components thereof; sorbitan esters (Spans®) such as Span 20 (sorbitan monolaurate), Span 40 (sorbitan monopalmitate), Span 60 (sorbitan monostearate), Span 80 (sorbitan monooleate); amphiphilic block copolymers; cholesterol; egg yolk- and soy-derived phosphatidylcholines; cyclodextrins such as 2-hydroxypropyl-β-cyclodextrin; lecithin; or any combination thereof.

Clause 27: The food additive of any one of Clauses 23 to 26, wherein the microencapsulation system comprises emulsions, nanoemulsions, micelles, solid lipid nanoparticles, nanostructured lipid carriers, liposomes, nanoliposomes, niosomes, polymer particles, hydrogel particles, or combinations thereof.

Clause 28: The food additive of Clause 27, wherein the microencapsulation system comprises emulsions and/or nanoemulsions.

Clause 29: The food additive of Clause 28, wherein the encapsulating agent is an emulsifier, and optionally further comprises at least one of a weighting agent, a ripening inhibitor, and a texture modifier.

Clause 30: The food additive of Clause 29, wherein the emulsifier is a polysaccharide-based emulsifier, a protein-based emulsifier, a small-molecule surfactant, or a mixture thereof.

Clause 31: The food additive of any one of Clauses 28 to 30, wherein the emulsions and/or nanoemulsions are prepared using a homogenizer.

Clause 32: The food additive of any one of Clauses 28 to 30, wherein the emulsions and/or nanoemulsions are prepared using a spontaneous emulsification method, an emulsion inversion point method, and/or a phase inversion temperature method.

Clause 33: The food additive of any one of Clauses 1 to 32, further comprising an antidote of the cannabinoid.

Clause 34: The food additive of Clause 33, wherein the cannabinoid is THC, and the antidote of THC comprises at least one of CBD; Acorus calamus or an extract thereof; black pepper or an extract thereof; citrus or an extract thereof; pine nuts or an extract thereof; pistachio nuts or an extract thereof; fruits of Pistacia terebinthus or an extract thereof; piperine; and terpenes, such as β-caryophyllene, limonene, myrcene, and α-pinene.

Clause 35: The food additive of Clause 34, wherein the antidote and the THC are each encapsulated in a respective microencapsulation system that is different one from the other.

Clause 36: The food additive of Clause 35, wherein the microencapsulation system of THC comprises particles having an average size of less than about 100 nm, and the microencapsulation system of the antidote comprises particles having an average size of more than about 100 nm.

Clause 37: The food additive of any one of Clauses 23 to 36, wherein the beverage product is stored in a container comprising a de-emulsification agent that can be released into the beverage product.

Clause 38: The food additive of Clause 37, wherein the de-emulsification agent is selected from: acids selected from succinic acid, fumaric acid, and citric acid; bases selected from sodium carbonate, potassium carbonate, sodium hydroxide, and potassium hydroxide; alcohols selected from ethanol and glycerol; electrolytes selected from sodium sulfate, sodium chloride, and the aforementioned acids and bases; and enzymes selected from cellulase, protease, amylase, and lipase.

Clause 39: A beverage comprising the food additive of any one of clauses 1 to 39.

Clause 40: An edible product comprising the food additive as described herein.

Clause 41: The edible product of clause 41, which is a beverage.

### Clause Set 3

Clause 1: A beverage comprising at least 0.002 mg/ml of a cannabinoid, the beverage having a turbidity of less than 0.05 cm⁻¹ at 600 nm.

Clause 2: A beverage in a packaging unit, the unit comprising less than 1000 mg, or less than 900 mg, or less than 800 mg, or less than 700 mg, or less than 600 mg, or less than 500 mg, or less than 400 mg, or less than 300 mg, or less than 200 mg, or less than 100 mg, or less than 50 mg, or less than 40 mg, or less than 30 mg, or less than 20 mg, or less than 10 mg, or less than 5 mg, or less than 2.5 mg of cannabinoid, the beverage having a turbidity of less than 0.05 cm⁻¹ at 600 nm.

Clause 3: The beverage of clause 1 or 2, having a viscosity selected in the range of from 50 mPas to 1500 mPas.

Clause 4: The beverage of any one of clauses 1 to 3, wherein the beverage is selected from drinking water, dairy milk, non-dairy milk, juice, a smoothie, coffee or a caffeinated beverage, tea, herbal tea, an energy drink, a fermented beverage, non-alcoholic beer, and a cocoa beverage.

Clause 5: The beverage of any one of clauses 1 to 3, wherein the beverage is selected from a carbonated drink and a nitrogenated drink.

Clause 6: The beverage of any one of clauses 1 to 3, wherein the beverage is an alcoholic beverage.

Clause 7: The beverage of clause 6, wherein the alcoholic beverage is selected from beer, lager, cider, spirit, wine/fortified wine, and cocktail.

Clause 8: The beverage of any one of clauses 1 to 7, wherein the cannabinoid includes tetrahydrocannabinol (THC).

Clause 9: The beverage of any one of clauses 1 to 8, further comprising a terpene.

Clause 10: The beverage of any one of clauses 1 to 9, wherein the cannabinoid includes cannabidiol (CBD).

Clause 11: The beverage of any one of clauses 1 to 7, wherein the cannabinoid includes tetrahydrocannabinol (THC) and cannabidiol (CBD) in a ratio of 1:1.

Clause 12: A process for obtaining a cannabinoid beverage, comprising (a) blending the cannabinoid beverage with a fining agent under fining conditions, and (b) recovering the cannabinoid beverage obtained after step a).

Clause 13: The process of clause 12, wherein the fining agent includes gelatin.

Clause 14: The process of clause 13, wherein the gelatin is blended with the beverage at a concentration of ≤2% (wt./wt.).

Clause 15: The process of clause 14, wherein the gelatin is blended with the beverage at a concentration of ≤1% (wt./wt.).

Clause 16: The process of clause 14, wherein the gelatin is blended with the beverage at a concentration of ≤0.8% (wt./wt.).

Clause 17: The process of clause 14, wherein the gelatin is blended with the beverage at a concentration of ≥0.05% (wt./wt.), or ≥0.1% (wt./wt.), or ≥0.2% (wt./wt.), or ≥0.3% (wt./wt.), or ≥0.4% (wt./wt.), or ≥0.5% (wt./wt.), or ≥0.6% (wt./wt.), or ≥0.7% (wt./wt.).

Clause 18: The process of clause 14, wherein the gelatin is blended with the beverage at a concentration included in the range of 0.8% to 1% (wt./wt.).

Clause 19: The process of any one of clauses 12 to 18, wherein the fining conditions include storing the blend obtained in a) at a temperature of ≤4° C. for at least 1 h.

Clause 20: The process of any one of clauses 12 to 19, further comprising: c) incorporating the beverage into a beverage packaging.

### Clause Set 4

Clause 1: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product having a turbidity of less than 0.05 cm−1 at 600 nm.

Clause 2: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product having a viscosity selected in the range of from 50 mPas to 1500 mPas.

Clause 3: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product having an odor index which is substantially the same as that one of the cannabinoid-less beverage.

Clause 4: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product having a tasting index which is substantially the same as that one of the cannabinoid-less beverage.

Clause 5: A food additive comprising an emulsion of a cannabinoid, wherein dilution or infusion of the food additive in a cannabinoid-less beverage or blending with a beverage base results in a beverage product comprising at least 0.002 mg/ml of cannabinoid in volume of the beverage product, the beverage product being stable for at least 1 month at 4° C.

Clause 6: A food additive comprising an emulsion of a cannabinoid which is at least partially miscible in water such that when the emulsion comprises 30 ml/ml of cannabinoids, dilution or infusion of the food additive in a cannabinoid-less beverage or blending with a beverage base results in a beverage product comprising at least 0.002 mg/ml of cannabinoid in volume of the beverage product.

Clause 7: The food additive of any one of Clauses 1 to 6, wherein the beverage product is selected from drinking water, dairy milk, non-dairy milk, juice, a smoothie, coffee or a caffeinated beverage, tea, herbal tea, an energy drink, a fermented beverage, non-alcoholic beer, and a cocoa beverage.

Clause 8: The food additive of any one of Clauses 1 to 6, wherein the beverage product is selected from a carbonated drink and a nitrogenated drink.

Clause 9: The food additive of any one of Clauses 1 to 6, wherein the beverage product is an alcoholic beverage.

Clause 10: The food additive of Clause 9, wherein the alcoholic beverage is selected from beer, lager, cider, spirit, wine/fortified wine, and cocktail.

Clause 11: The food additive of any one of Clauses 1 to 10, wherein the emulsion includes tetrahydrocannabinol (THC).

Clause 12: The food additive of any one of Clauses 1 to 10, wherein the emulsion includes cannabidiol (CBD).

Clause 13: The food additive of any one of Clauses 1 to 10, wherein the emulsion includes a terpene.

Clause 14: The food additive of any one of Clauses 1 to 10, wherein the emulsion includes tetrahydrocannabinol (THC) and cannabidiol (CBD).

Clause 15: The food additive of Clause 14, wherein the THC and CBD are present in a ratio of 1:1.

Clause 16: The food additive of any one of Clauses 1 to 15, in the form of a powder.

Clause 17: The food additive of any one of Clauses 1 to 15, in the form of a liquid.

Clause 18: The food additive of any one of Clauses 1 to 15, in the form of a capsule, lozenge or tablet.

Clause 19: The food additive of any one of Clauses 1 to 18, wherein the additive has less than 100,000 CFU of total viable aerobic bacteria count.

Clause 20: The food additive of any one of Clauses 1 to 19, wherein the additive has less than 100,000 CFU/g of total yeast and mold count, preferably less than 10,000 CFU/g.

Clause 21: The food additive of any one of Clauses 1 to 20, wherein the additive has less than 1000 CFU of bile-tolerant gram negative bacteria.

Clause 22: The food additive of any one of Clauses 1 to 21, wherein the additive has less than 1000 CFU/g of total coliforms count, preferably less than 100 CFU/g.

Clause 23: The food additive of any one of Clauses 1 to 22, comprising an encapsulating agent that forms a microencapsulation system with the cannabinoid.

Clause 24: The food additive of Clause 23, wherein the encapsulating agent is a film-forming natural or synthetic biopolymer, a small-molecule surfactant, or a combination thereof.

Clause 25: The food additive of Clause 24, wherein the biopolymer is a protein, a carbohydrate, a lipid, a fat, or a gum.

Clause 26: The food additive of Clause 24, wherein the encapsulating agent is arabic gum; starches such as corn starch; modified starches such as octenyl succinate modified starches; modified cellulose such as methyl cellulose, hydroxypropyl cellulose, methyl hydroxypropyl cellulose, and carboxymethylcellulose; certain types of pectin such as beet pectin; polysaccharides such as maltodextrin and soy soluble polysaccharides; corn fiber gum; globular proteins such as whey protein and whey protein ingredients such as whey protein concentrate, whey protein isolate, and highly purified protein fractions such as β-lactoglobulin and α-lactalbumin; flexible proteins such as gelatin and caseins such as sodium caseinate, calcium caseinate, and purified protein fractions such as β-casein; Tweens® (polysorbates) such as Tween 20 (polyoxyethylene sorbitan monolaurate), Tween 40 (polyoxyethylene sorbitan monopalmitate), Tween 60 (polyoxyethylene sorbitan monostearate), Tween 40 (polyoxyethylene sorbitan monopalmitate), Tween 60 (polyoxyethylene sorbitan monostearate), and Tween 80 (polyoxyethylene sorbitan monooleate); sugar esters such as sucrose monopalmitate, sucrose monostearate, sucrose distearate, sucrose polystearate, and sucrose laurate; quillaja saponin (Q-Naturale®) and components thereof; sorbitan esters (Spans®) such as Span 20 (sorbitan monolaurate), Span 40 (sorbitan monopalmitate), Span 60 (sorbitan monostearate), Span 80 (sorbitan monooleate); amphiphilic block copolymers; cholesterol; egg yolk- and soy-derived phosphatidylcholines; cyclodextrins such as 2-hydroxypropyl-β-cyclodextrin; lecithin; or any combination thereof.

Clause 27: The food additive of any one of Clauses 23 to 26, wherein the microencapsulation system comprises emulsions, nanoemulsions, micelles, solid lipid nanoparticles, nanostructured lipid carriers, liposomes, nanoliposomes, niosomes, polymer particles, hydrogel particles, or combinations thereof.

Clause 28: The food additive of Clause 27, wherein the microencapsulation system comprises emulsions and/or nanoemulsions.

Clause 29: The food additive of Clause 28, wherein the encapsulating agent is an emulsifier, and optionally further comprises at least one of a weighting agent, a ripening inhibitor, and a texture modifier.

Clause 30: The food additive of Clause 29, wherein the emulsifier is a polysaccharide-based emulsifier, a protein-based emulsifier, a small-molecule surfactant, or a mixture thereof.

Clause 31: The food additive of any one of Clauses 28 to 30, wherein the emulsions and/or nanoemulsions are prepared using a homogenizer.

Clause 32: The food additive of any one of Clauses 28 to 30, wherein the emulsions and/or nanoemulsions are prepared using a spontaneous emulsification method, an emulsion inversion point method, and/or a phase inversion temperature method.

Clause 33: The food additive of any one of Clauses 1 to 32, further comprising an antidote of the cannabinoid.

Clause 34: The food additive of Clause 33, wherein the cannabinoid is THC.

Clause 35: The food additive of Clause 34, wherein the antidote of THC comprises at least one of CBD; Acorus calamus or an extract thereof; black pepper or an extract thereof; citrus or an extract thereof; pine nuts or an extract thereof; pistachio nuts or an extract thereof; fruits of Pistacia terebinthus or an extract thereof; piperine; and terpenes, such as β-caryophyllene, limonene, myrcene, and α-pinene.

Clause 36: The food additive of Clause 35, wherein the antidote and the THC are each encapsulated in a respective microencapsulation system that is different one from the other.

Clause 37: The food additive of Clause 36, wherein the microencapsulation system of THC comprises particles having an average size of less than about 100 nm, and the microencapsulation system of the antidote comprises particles having an average size of more than about 100 nm.

Clause 38: The food additive of any one of Clauses 23 to 37, wherein the beverage product is stored in a container comprising a de-emulsification agent that can be released into the beverage product.

Clause 39: The food additive of Clause 38, wherein the de-emulsification agent is selected from: acids selected from succinic acid, fumaric acid, and citric acid; bases selected from sodium carbonate, potassium carbonate, sodium hydroxide, and potassium hydroxide; alcohols selected from ethanol and glycerol; electrolytes selected from sodium sulfate, sodium chloride, and the aforementioned acids and bases; and enzymes selected from cellulase, protease, amylase, and lipase.

Clause 40: A beverage comprising the food additive of any one of clauses 1 to 39.

Clause 41: An edible product comprising the food additive as described herein.

Clause 42: The edible product of clause 41, which is a beverage.

Clause 43: A beverage comprising a cannabinoid and an emulsifier.

Clause 44: The beverage of clause 43, which is tea or herbal tea.

Clause 45: The beverage of clause 43, which is coffee or a caffeinated beverage.

Clause 46: The beverage of clause 43, which is a carbonated drink or a nitrogenated drink.

Clause 47: The beverage of clause 43, which is an energy drink.

Clause 48: The beverage of clause 43, which is an alcoholic drink, such as beer, lager, cider, spirit, wine/fortified wine, and cocktail.

Clause 49: The beverage of any one of clauses 43 to 48, wherein the cannabinoid is tetrahydrocannabinol (THC).

Clause 50: The beverage of any one of clauses 43 to 48, wherein the cannabinoid is cannabidiol (CBD).

Clause 51: The beverage of any one of clauses 43 to 48, wherein the cannabinoid is a mixture of tetrahydrocannabinol (THC) and cannabidiol (CBD).

Clause 52: The beverage of clause 51, wherein the ratio of THC to CBD in the liquid formulation is about 1:1.

Clause 53: The beverage of any one of clauses 43 to 52, wherein the emulsifier is a polysaccharide-based emulsifier, a protein-based emulsifier, a small-molecule surfactant, or a mixture thereof.

Clause 54: The beverage of clause 53, wherein the emulsifier is gum arabic, modified starches such as octenyl succinate modified starches, modified cellulose such as methyl cellulose, hydroxypropyl cellulose, methyl hydroxypropyl cellulose, and carboxymethylcellulose, certain types of pectin such as beet pectin, soy soluble polysaccharide, corn fiber gum, or a mixture thereof.

Clause 55: The beverage of clause 53, wherein the emulsifier is a globular protein such as whey protein and whey protein ingredients such as whey protein concentrate, whey protein isolate, and highly purified protein fractions such as β-lactoglobulin and α-lactalbumin; a flexible protein such as gelatin and caseins such as sodium caseinate, calcium caseinate, and purified protein fractions, such as β-casein; or a mixture thereof.

Clause 56: The beverage of clause 53, wherein the emulsifier is a Tween (polysorbate) such as Tween 20 (polyoxyethylene sorbitan monolaurate), Tween 40 (polyoxyethylene sorbitan monopalmitate), Tween 60 (polyoxyethylene sorbitan monostearate), and Tween 80 (polyoxyethylene sorbitan monooleate); a sugar ester such as sucrose monopalmitate, sucrose monostearate, sucrose distearate, sucrose polystearate, quillaja saponin (Q-Naturale®) and components thereof; a sorbitan ester (Span) such as Span 20 (sorbitan monolaurate), Span 40 (sorbitan monopalmitate), Span 60 (sorbitan monostearate), and Span 80 (sorbitan monooleate); or a mixture thereof.

Clause 57: The beverage of any one of clauses 43 to 56, further comprising at least one of a weighting agent, a ripening inhibitor, and a texture modifier.

Clause 58: The beverage of any one of clauses 43 to 57, which comprises at least 0.002 mg/ml of the cannabinoid in volume of the beverage and has a turbidity of less than 0.05 cm−1 at 600 nm.

Clause 59: The beverage of any one of clauses 43 to 57, which comprises at least 0.002 mg/ml of the cannabinoid in volume of the beverage and has a viscosity selected in the range of from 50 mPas to 1500 mPas.

Clause 60: The beverage of any one of clauses 43 to 57, which comprises at least 0.002 mg/ml of the cannabinoid in volume of the beverage and has an odor index which is substantially the same as that one of the cannabinoid-less beverage.

Clause 61: The beverage of any one of clauses 43 to 57, which comprises at least 0.002 mg/ml of the cannabinoid in volume of the beverage and has a tasting index which is substantially the same as that one of the cannabinoid-less beverage.

Clause 62: The beverage of any one of clauses 43 to 57, which comprises at least 0.002 mg/ml of the cannabinoid in volume of the beverage and is stable for at least 1 month at 4° C.

Clause 63: An emulsifying system comprising a cannabinoid and an emulsifier.

Clause 64: The emulsifying system of clause 63, which in the form of a powder.

Clause 65: The emulsifying system of clause 63, which in the form of a liquid.

Clause 66: The emulsifying system of clause 63, which in the form of a lyophilisate.

Clause 67: The emulsifying system of clause 63, which in the form of a gel.

Clause 68: The emulsifying system of clause 63, which in the form of a gum.

Clause 69: The emulsifying system of any one of clauses 63 to 68, wherein the cannabinoid is embedded in the emulsifier.

Clause 70: The emulsifying system of any one of clauses 63 to 68, wherein the cannabinoid is encapsulated in the emulsifier.

Clause 71: The emulsifying system of any one of clauses 63 to 68, wherein the cannabinoid is dispersed in the emulsifier.

Clause 72: The emulsifying system of any one of clauses 63 to 71, wherein the cannabinoid is tetrahydrocannabinol (THC).

Clause 73: The emulsifying system of any one of clauses 63 to 71, wherein the cannabinoid is cannabidiol (CBD).

Clause 74: The emulsifying system of any one of clauses 63 to 71, wherein the cannabinoid is a mixture of tetrahydrocannabinol (THC) and cannabidiol (CBD).

Clause 75: The emulsifying system of clause 74, wherein the ratio of THC to CBD in the liquid formulation is about 1:1.

Clause 76: The emulsifying system of any one of clauses 63 to 75, wherein the emulsifier is a polysaccharide-based emulsifier, a protein-based emulsifier, a small-molecule surfactant, or a mixture thereof.

Clause 77: The emulsifying system of clause 76, wherein the emulsifier is gum arabic, modified starches such as octenyl succinate modified starches, modified cellulose such as methyl cellulose, hydroxypropyl cellulose, methyl hydroxypropyl cellulose, and carboxymethylcellulose, certain types of pectin such as beet pectin, soy soluble polysaccharide, corn fiber gum, or a mixture thereof.

Clause 78: The emulsifying system of clause 76, wherein the emulsifier is a globular protein such as whey protein and whey protein ingredients such as whey protein concentrate, whey protein isolate, and highly purified protein fractions such as β-lactoglobulin and α-lactalbumin; a flexible protein such as gelatin and caseins such as sodium caseinate, calcium caseinate, and purified protein fractions, such as β-casein; or a mixture thereof.

Clause 79: The emulsifying system of clause 76, wherein the emulsifier is a Tween (polysorbate) such as Tween 20 (polyoxyethylene sorbitan monolaurate), Tween 40 (polyoxyethylene sorbitan monopalmitate), Tween 60 (polyoxyethylene sorbitan monostearate), and Tween 80 (polyoxyethylene sorbitan monooleate); a sugar ester such as sucrose monopalmitate, sucrose monostearate, sucrose distearate, sucrose polystearate, quillaja saponin (Q-Naturale®) and components thereof; a sorbitan ester (Span) such as Span 20 (sorbitan monolaurate), Span 40 (sorbitan monopalmitate), Span 60 (sorbitan monostearate), and Span 80 (sorbitan monooleate); or a mixture thereof.

Clause 80: The emulsifying system of any one of clauses 63 to 79, further comprising at least one of a weighting agent, a ripening inhibitor, and a texture modifier.

Clause 81: The beverage of any one of clauses 43 to 62, or the emulsifying system of any one of clauses 63 to 80, which comprises the cannabinoid in an amount of 1 mg, 5 mg, 10 mg, 50 mg, or 100 mg.

### EXAMPLES

The following examples describe some exemplary modes of making and practicing certain compositions that are described herein. It should be understood that these examples are for illustrative purposes only and are not meant to limit the scope of the compositions and methods described herein.

### Example 1

In this example, compositions containing an emulsion having particle sizes >1000 nm (Formulation 1), 200 nm (Formulation 2) and 40 nm (Formulation 3) were made.

Cannabinoid based emulsions having a particle size of 40 nm and 200 nm are provided below in Tables 1 and 2. Cannabinoid based emulsions having a particle size of >1000 nm were prepared based on the formulae set out in Tables 1 and 2, without the additional sonication step.

These exemplary formulations span the range from nanoemulsions to macroemulsions. The foregoing emulsions were prepared as follows:
1. The water and oil phase ingredients were solubilized separately using heat and stirring. In particular, the water phase is comprised of water, Tween™ 80, ascorbic acid and EDTA and mixed at 60° C. with a magnetic stir bar for 30 minutes. The oil phase is comprised of Labrafac™ lipophile WL 1349, Tocobiol™, lecithin and THC distillate and mixed at 60° C. with a magnetic stir bar for 30 minutes.
2. Once the respective water and oil phases have been prepared they were combined while mixing with a high shear homogenizer at 8000-10000 rpm. The oil phase was added slowly to the water phase over 5 minutes and once completely the resultant emulsion was mixed for an additional 15 minutes. The resultant mixture is a macro-emulsion with a particle size >1000 nm.
3. To generate the 40 nm and 200 nm nano-emulsions, high energy sonication was applied to the macro-emulsions for 10 minutes with 100% amplitude using an ISP-500 Ultrasonic Processor (Sonomechanics, Florida, USA).

Using the same excipient components and tuning the ratio of emulsifiers to achieve the different particle sizes eliminates the experimental uncertainty in permeation data (see later example) interpretation that would normally be associated if using different emulsifier combinations to achieve the different particle sizes.

Particle size of all nanoemulsions was measured in water solution at 25° C. using dynamic light scattering (DLS). All samples in the present disclosure have been analyzed at a dilution of 1/20 in purified water using a LiteSizer™ (Anton Paar GmbH, Germany).

TABLE 1

| Excipients | Mass (g) | % Blend |
|---|---|---|
| THC Distillate-03 | 18.75 | 2.5 |
| Labrafac lipophile | 20 | 2.67 |
| Ascorbic acid | 4.5 | 0.6 |
| Tocobiol | 3.75 | 0.5 |
| EDTA | 0.1 | 0.01 |
| Lecithin | 15 | 2 |
| Tween 80 | 60 | 8 |
| Water | 627.9 | 83.72 |

TABLE 2

| Excipients | Mass (g) | % Blend |
|---|---|---|
| THC Distillate-03 | 18.75 | 2.5 |
| Labrafac lipophile | 20 | 2.67 |
| Ascorbic acid | 3.75 | 0.5 |
| Tocobiol | 4.5 | 0.6 |
| EDTA | 0.1 | 0.01 |
| Lecithin | 10 | 1.33 |
| Tween80 | 15 | 2 |
| Water | 677.9 | 90.39 |

The results clearly demonstrate that the emulsification approach of the present disclosure allows for tuning the ratio of the emulsifiers to achieve different particle sizes suitable for formulating with a variety of product bases. Additionally, it eliminates the experimental uncertainty that would normally be associated with using different emulsifier combinations to achieve different particle sizes.

### Example 2

In this example, a composition containing THC with a particle size ≤100 nm was made.

1,000 mg of THC-containing cannabis oil was mixed with 50 mg of poly(ethylene glycol) monooleate with an appropriate amount of ethanol in a container to obtain an oil phase mixture. The oil phase mixture was heated at 50° C. until a liquid oil phase was obtained. In a separate container, 50 mg of sodium oleate were dissolved into 20 mL of deionized water to form an aqueous phase mixture. The oil phase mixture was added to the aqueous phase mixture and the combined mixture was mixed with a high shear mixer to obtain a coarse emulsion. A T25 (IKA, Staufen, Germany) at 8,000 rpm for 5 minutes can be used here. The coarse emulsion was mixed with a microfluidizer to further homogenize the emulsion and obtain the first composition containing THC with a particle size ≤100 nm. A Nano DeBEE, (Westwood, Mass., USA) at 20,000 psi for 8-12 cycles can be used here.

### Example 3

In this example, a composition containing CBD with a PSD of about 200 nm was made.

5 g of limonene and 25 g of whey protein isolate were mixed with 70 g of water by stirring. The mixture was left for 24 hours to allow complete biopolymer hydration and saturation. After 24 hours, the mixture was homogenized using a sonicator. A Digital Sonifier 450 (Branson Ultrasonic Corporation, USA) at 160 W for 2 minutes can be used here. After homogenization, the emulsion was placed in an ice bath until the emulsion reached room temperature so as to obtain the second composition containing CBD with a PSD of ≥200 nm.

### Example 4

In this example, a composition containing CBD with a PSD of ≥200 nm was made.

5 g of CBD-containing cannabis oil extract was mixed with 0.794 g Tween 80, 4.206 g Span 80, and 90 g distilled water in a test tube. The resulting mixture was heated to 70° C. and immediately homogenized to obtain the second composition containing CBD with a PSD of ≥200 nm. An Ultra Turrax T 25 device (IKA, Staufen, Germany) at 13,400 rpm for 15 minutes can be used here.

### Example 5

In this example, a composition containing CBD with a PSD of ≥200 nm was made.

0.794 g Tween 80 was dissolved in 90 g distilled water to form an aqueous phase. 4.206 g Span 80 was dissolved in 5 g CBD cannabis oil to form an oil phase. Both the aqueous and oil phases were heated to 70° C. and maintained at this temperature. The aqueous phase was added drop-wise to the oil phase, while stirring the oil phase to obtain the composition containing CBD with a PSD of ≥200 nm. An RZR Heidolph homogenizer (Heidolph Instruments GmbH & Co. KG, Schwabach, Germany) at 1050 rpm over a duration of 30 min can be used here.

### Example 6

In this example, a composition containing CBD with a PSD of ≥200 nm was made.

The same procedure as described in Example 5 was repeated except that 1.262 g Tween 80 was dissolved in 90 g distilled water to form the aqueous phase and 3.738 g Span 80 was dissolved in 5 g CBD cannabis oil extract to form the oil phase.

### Example 7

In this example, a composition containing CBD with a PSD of ≥200 nm was made.

The same procedure as described in Example 5 was repeated except that 1.729 g Tween 80 was dissolved in 90 g distilled water to form the aqueous phase and 3.271 g Span 80 was dissolved in 5 g CBD cannabis oil extract to form the oil phase.

### Example 8

In this example, a composition containing CBD with a PSD of ≥200 nm was made.

The same procedure as described in Example 5 was repeated except that 2.196 g Tween 80 was dissolved in 90 g distilled water to form the aqueous phase and 2.804 g Span 80 was dissolved in 5 g CBD cannabis oil extract to form the oil phase.

### Example 9

In this example, a composition containing CBD with a PSD of ≥200 nm was made.

The same procedure as described in Example 5 was repeated except that 2.664 g Tween 80 was dissolved in 90 g distilled water to form the aqueous phase and 2.336 g Span 80 was dissolved in 5 g CBD cannabis oil extract to form the oil phase.

### Example 10

In this example, a composition containing CBD with a PSD of ≥200 nm was made.

The same procedure as described in Example 5 was repeated except that 2.826 g Tween 80 was dissolved in 90 g distilled water to form the aqueous phase and 2.174 g Span 80 was dissolved in 5 g CBD cannabis oil extract to form the oil phase.

### Example 11

In this example, a composition containing CBD with a PSD of ≥200 nm was made.

The same procedure as described in Example 5 was repeated except that 3.370 g Tween 80 was dissolved in 90 g distilled water to form the aqueous phase and 1.630 g Span 80 was dissolved in 5 g CBD cannabis oil extract to form the oil phase.

### Example 12

In this example, a composition containing CBD with a PSD of ≥200 nm was made.

The same procedure as described in Example 5 was repeated except that 3.913 g Tween 80 was dissolved in 90 g distilled water to form the aqueous phase and 1.087 g Span 80 was dissolved in 5 g CBD cannabis oil extract to form the oil phase.

### Example 13—Mucolytic Agent

In this example, a composition containing THC and a mucolytic agent was made.

Kollipor EL (30% w/w) as surfactant and propylene glycol (47% w/w) as co-solvent were mixed with THC (3% w/w) at 40° C. for 30 minutes using a magnetic stirrer (Hotplate Stirrer Stuart) at the rate of 200 rpm. Captex 355 as oil (20% w/w) was added to this mixture and stirred for a further 30 min at 40° C. at 500 rpm. This mixture was dispersed in 0.1 M phosphate buffered saline solution (pH 6.8) with a volume ratio of 1:100 by stirring at 50 rpm. Papain-palmitate was dispersed in oleic acid at a concentration of 10% (m/v), and subsequently, equal volume of papain-palmitate dispersion and phosphate-buffered mixture were mixed at vortex for 10 min followed by sonication for 6 h at room temperature using Bandelin Sonorex at a frequency of 35 kHz. Droplet-sized particles were immediately observed after dispersing in 0.1 M phosphate buffer solution (pH 6.8) at a volume ratio of 1:100.

Papain-palmitate was prepared according to the following procedure:

Papain was dissolved in 0.1 M phosphate buffer (pH 8.0) at a concentration of 3 mg/ml using a thermomixer. Palmitoyl chloride solution in acetone at a concentration of 100 mg/ml was added dropwise into the papain solution at a volume ratio of 1:40. The pH was maintained at 8 by addition of 1 M NaOH. The reaction was conducted for 90 min at room temperature and produced a suspension. Afterwards, the modified papain suspension was dialyzed against water for 24 h followed by lyophilization.

This procedure for incorporating a mucolytic agent can be performed with any of the compositions described in the examples.

### Example 14—Efflux Blocker

In this example, a composition containing a cannabinoid and an efflux blocker was made.

504 mg of polysorbate 20, 504 mg of sorbitan monoleate, 504 mg of polyoxyl 40-hydroxy castor oil, and 504 mg of tricaprin were mixed in a container. In a separate container, 996 mg of ethyl lactate and 254 mg of lecithin were mixed and heated to 40° C. in a scintillation tube until complete dissolution. Both mixtures were mixed together using gentle stirring. The combined mixture was heated to 40° C. until a homogenous pre-concentrate solution was formed. 103 mg of cannabis oil was added to the pre-concentrate solution. The combined mixture was stirred gently, where upon gentle agitation of the cannabinoid in the aqueous phase, the pre-concentrate spontaneously forms drug encapsulated O/W nano-dispersion. 69 mg of an efflux blocker was added to form an advanced pro-nanoparticulates and the mixture was heated to 40° C. until a homogenous solution was formed.

This procedure for incorporating an efflux blocker can be performed with any of the compositions described in the examples.

### Example 15

In this example, various compositions containing THC at 2.5 wt. % were made in accordance with embodiments of the present disclosure and as per the procedure set forth in Example 1.

TABLE 3

| Ingredient | Mass (g) | % Blend |
|---|---|---|
| THC Distillate | 3.75 | 2.50 |
| Coconut Oil | 4 | 2.67 |
| Lecithin sunflower | 3 | 2 |
| Tween 80 | 12 | 8 |
| Water | 127.25 | 84.83 |

| | |
|---|---|
| PSD | 59.4 nm |

TABLE 4

| Ingredients | Mass (g) | % Blend |
|---|---|---|
| THC Distillate | 3.75 | 2.50 |
| Coconut Oil | 4.00 | 2.67 |
| Span 80 | 3.00 | 2.00 |
| Tween 80 | 12.00 | 8.00 |
| Water | 127.25 | 84.83 |

| | |
|---|---|
| PSD | 122.7 nm |

TABLE 5

| Ingredients | Mass (g) | % Blend |
|---|---|---|
| THC Distillate | 3.75 | 2.50 |
| Coconut Oil | 4.00 | 2.67 |
| Brij ™ C2-SO | 1.50 | 1.00 |
| Tween 80 | 11.00 | 7.33 |
| Water (g): | 129.75 | 86.50 |

| | |
|---|---|
| PSD | 87.4 nm |

TABLE 6

| Ingredients | Mass (g) | % Blend |
|---|---|---|
| THC Distillate | 3.75 | 2.50 |
| Coconut Oil | 4.00 | 2.67 |
| Vit E TPGS | 3.00 | 2.00 |
| Tween 80 | 9.00 | 6.00 |
| Lecithin sunflower | 3.00 | 2.00 |
| Water | 127.25 | 84.83 |

| | |
|---|---|
| PSD | 36 nm |

### Example 16—Precursor Composition

In this example, a precursor composition in accordance with an embodiment of the present disclosure was made by gently mixing a first composition containing THC with a particle size ≤100 nm (as described in any of the previous examples) and a second composition containing CBD with a particle size >200 nm (as described in any of the previous examples).

The first composition and the second composition were gently mixed to obtain a precursor composition in accordance with an embodiment of the present disclosure.

### Example 17

A THC precursor composition obtained as per the procedure set out in Example 16 was incorporated into a beverage base to obtain a cannabis-infused beverage which was canned into a container (e.g., 355 ml can) so as to include 10 mg THC and 10 mg CBD per container in accordance with an embodiment of the present disclosure. This beverage thus contained a fast onset portion with a cannabinoid profile and a delayed onset portion with an antidote, modulator or attenuator of the cannabinoid profile.

### Example 18

In this example, the Franz cell test was used to evaluate the behavior of a liquid composition comprising a 20 mg/ml THC emulsion made as per Example 1.

In this test, the biological membranes included in the Franz cell test were obtained from freshly slaughtered pigs and 1 ml of the 20 mg/ml THC emulsion was loaded into the donor cell.

FIG. 3 shows that surprisingly and unexpectedly, as particle size is decreased permeation of cannabinoids through the membrane increases significantly. FIG. 3 shows that the emulsion having a PSD of 40 nm exhibits a significantly greater accumulated concentration than all other samples having a THC concentration about 3× greater than that of the 200 nm emulsion and about 32× greater than the >1000 nm emulsion. This information is highly relevant to pre-clinical study development, and the inventors predict therefrom that a highly permeable vehicle such as the 40 nm emulsion would be expected to be more rapidly absorbed in vivo generating a fast Tmax and fast onset of cannabinoid experience compared to a 200 nm emulsion or the >1000 nm emulsion.

Comparing the 200 nm to the >1000 nm emulsions, FIG. 3 again shows a similar trend whereby the 200 nm emulsion shows a significantly faster permeation across the biological membrane. In fact the 200 nm emulsion shows an accumulated concentration almost 10× greater than the >1000 nm emulsion. This further supports the hypothesis that as emulsion particle size is decreased permeation of cannabinoids is increased. More importantly, these results suggest that by using formulations which generate different particle sizes, absorption and onset can be accurately controlled for the user experience.

### Example 19

Beverages were obtained by blending a precursor composition into a beverage base. The emulsion was obtained using polysorbates (Tween-20) and Tween-80 to emulsify crude cannabis resin (CBD-resin) into a selection of 12 beverage bases.

Different mixing methods were used and tested:
1. Mix the resin and Tween-20 or Tween-80 at room temperature
2. Mix the resin and Tween-20 or Tween-80 at room temperature, and sonicate the mixture
3. Mix the resin and Tween-20 or Tween-80 in water bath at 35° C., and sonicate the mixture.

Ratios of resin to surfactant of 1:3, 1:5 and 1:10 resulted in a homogenised mixture. The mixture of the surfactant and the resin was added to a beverage base by sonication with the exception of manual mixing for a carbonated beverage. Lab results confirm successful emulsification.

Cyclodextrin and the resin in a weight to weight ratio of 1:1 was manually mixed. The amount of cyclodextrin was increased until cyclodextrin was able to absorb all of the cannabis resin into the powder. This powder was then mixed with a base beverage using the same methods.

Lab results confirm successful emulsification, and the cannabis powder contained 11.4 wt. % THC.

The above beverages were processed using a fining agent under fining conditions to improve the clarity of the beverages, e.g., to obtain a turbidity of less than 0.05 cm⁻¹ at 600 nm.

Several fining agents and fining conditions were used. In an illustrative example, gelatin was used at various concentrations (wt./wt. %) from 0.7 wt. % up to 120 wt. %. The beverages were stored at 4° C. for at least 3 days, then processed with gelatin, and returned to storage at 4° C. for another 4 days.

There was heavy settlement in the beverages demonstrating that the fining procedure was complete. The clearest of the set was decanted. The lab results confirm that processing cannabinoid containing beverages using a fining agent under fining conditions improved the turbidity of the beverages so as to obtain a turbidity of less than 0.05 cm⁻¹ at 600 nm.

For example, it was observed that preferably one should use a concentration of ≤2% (wt./wt.) of gelatin in the cannabinoid-containing beverage so as to minimize settlement of the cannabinoid and emulsifying agent. For example, using 0.8 wt. %-1.0 wt. % gelatin produced a clear solution without affecting the cannabinoids content.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

Although the foregoing invention has been described in some detail by way of illustration and example for purposes of clarity of understanding, it is readily apparent to those of ordinary skill in the art in light of the teachings of this invention that certain changes and modifications may be made thereto without departing from the scope of the appended claims.

It is to be understood that any numerical value inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used herein, whether in the specification or the appended claims, the transitional terms “comprising”, “including”, “carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the invention disclosed and/or claimed herein.

1.-148. (canceled)

149. A cannabis-infused product, comprising a cannabinoid profile including one or more cannabinoid, a first composition for controlling onset of the cannabinoid profile and a second composition for extending offset of the cannabinoid profile in a subject having used the cannabis-infused product, wherein the second composition has a delayed onset compared to that one of the first composition.

150. The cannabis-infused product according to claim 149, the cannabinoid profile further including one or more terpene.

151. The cannabis-infused product according to claim 149, wherein the first and the second compositions are an emulsion.

152. The cannabis-infused product according to claim 151, the first composition comprising particles having a first particle size distribution (PSD1) and the second composition comprising particles having a second particle size distribution (PSD2), wherein PSD1<PSD2.

153. The cannabis-infused product according to claim 149, wherein the cannabinoid profile includes tetrahydrocannabinol (THC).

154. The cannabis-infused product according to claim 149, wherein the cannabinoid profile includes cannabidiol (CBD).

155. The cannabis-infused product according to claim 152, wherein the PSD1 is ≤200 nm.

156. The cannabis-infused product according to claim 152, wherein the PSD2 is >1000 nm.

157. The cannabis-infused product according to claim 149, wherein either or both the first and the second compositions include a film-forming biopolymer, an emulsifier, or a combination thereof.

158. The cannabis-infused product according to claim 149, further comprising a weighting agent, a ripening inhibitor, a texture modifier, or any combination thereof.

159. The cannabis-infused product according to claim 149, wherein the cannabis-infused product includes a non-liquid edible matrix.

160. The cannabis-infused product according to claim 159, wherein the product is selected from the group consisting of a baked good such as a muffin, cookie, or brownie; candy; gummy; chocolate; lozenge; chewing-gum; and chewing-gum having a hard shell.

161. The cannabis-infused product according to claim 149, wherein the cannabis-infused product is a cannabis-infused liquid composition.

162. The cannabis-infused product according to claim 161, wherein the product is selected from the group consisting of a nitrogenised beverage; carbonated beverage; drinking water; an energy drink; an alcoholic product; a non-alcoholic beverage such as non-alcoholic beer, lager, cider, spirit, wine or cocktail; and a fermented beverage.

163. A cannabis precursor composition for infusing a product base, the precursor composition comprising a cannabinoid profile including one or more cannabinoid, a first composition for controlling onset of the cannabinoid profile and a second composition for extending offset of the cannabinoid profile in a subject having used the cannabis-infused product, wherein the second composition has a delayed onset compared to that one of the first composition.

164. The cannabis precursor composition according to claim 163, the cannabinoid profile including one or more terpene.

165. The cannabis precursor composition according to claim 163, wherein the first and the second compositions are an emulsion.

166. The cannabis precursor composition according to claim 165, the first composition comprising particles having a first particle size distribution (PSD1) and the second composition comprising particles having a second particle size distribution (PSD2), wherein PSD1<PSD2.

167. The cannabis precursor composition according to claim 166, wherein the PSD1 is ≤200 nm.

168. The cannabis precursor composition according to claim 166, wherein the PSD2 is >1000 nm.

169. The cannabis precursor composition according to claim 163, wherein the cannabinoid profile includes tetrahydrocannabinol (THC).

170. The cannabis precursor composition according to claim 163, wherein the cannabinoid profile includes cannabidiol (CBD).

171. The cannabis precursor composition according to claim 163, wherein either or both the first and the second compositions include a film-forming biopolymer, an emulsifier, or a combination thereof.

172. The cannabis precursor composition according to claim 163, wherein the composition further comprises a weighting agent, a ripening inhibitor, a texture modifier, or any combination thereof.

173. The cannabis precursor composition according to claim 163, comprising up to 1 g/ml of the one or more cannabinoid per total volume of the precursor composition.

174. The cannabis precursor composition according to claim 163, for infusing a product base so as to obtain a non-liquid edible matrix cannabis-infused product

175. The cannabis precursor composition according to claim 174, which is in a dry form.

176. The cannabis precursor composition according to claim 174, wherein the product is selected from the group consisting of a baked good such as a muffin, cookie, or brownie; candy; gummy; chocolate; lozenge; chewing-gum; and chewing-gum having a hard shell.

177. The cannabis precursor composition according to claim 163, for infusing a product base so as to obtain a cannabis-infused liquid composition.

178. The cannabis precursor composition according to claim 177, wherein the product is selected from the group consisting of a nitrogenised beverage; carbonated beverage; drinking water; an energy drink; an alcoholic product; a non-alcoholic beverage such as non-alcoholic beer, lager, cider, spirit, wine or cocktail; and a fermented beverage.
